SEQUENCE LISTING (30 sequences)

SEQ ID NO: 1 - protein, 7 aa, synthetic construct
  REGION 1..7: note = HCDR1 of EDA-binding VH domain
GFTFSVM

SEQ ID NO: 2 - protein, 6 aa, synthetic construct
  REGION 1..6: note = HCDR2 of EDA-binding VH domain
SGSGGS

SEQ ID NO: 3 - protein, 9 aa, synthetic construct
  REGION 1..9: note = HCDR3 of EDA-binding VH domain
STHLYLFDY

SEQ ID NO: 4 - protein, 12 aa, synthetic construct
  REGION 1..12: note = LCDR1 of EDA-binding VL domain
RASQSVSNAFLA

SEQ ID NO: 5 - protein, 7 aa, synthetic construct
  REGION 1..7: note = LCDR2 of EDA-binding VL domain
GASSRAT

SEQ ID NO: 6 - protein, 9 aa, synthetic construct
  REGION 1..9: note = LCDR3 of EDA-binding VL domain
QQMRGRPPT

SEQ ID NO: 7 - protein, 118 aa, synthetic construct
  REGION 1..118: note = VH domain of EDA-binding moiety
EVQLLESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 8 - protein, 108 aa, synthetic construct
  REGION 1..108: note = VL domain of EDA-binding moiety
EIVLTQSPGTLSLSPGERATLSCRASQSVSNAFLAWYQQKPGQAPRLLIYGASSRATGIPDRFSGSGSGTDFTLTISRLEPEDFAVYYCQQMRGRPPTFGQGTKVEIK

SEQ ID NO: 9 - protein, 246 aa, synthetic construct
  REGION 1..246: note = anti-EDA F8 scFv
EVQLLESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGSGGGGSEIVLTQSPGTLSLSPGERATLSCRASQSVSNAFLAWYQQKPGQAPRLLIYGASSRATGIPDRFSGSGSGTDFTLTISRLEPEDFAVYYCQQMRGRPPTFGQGTKVEIK

SEQ ID NO: 10 - protein, 45 aa, Homo sapiens
  REGION 1..45: note = Hinge region of CD8
TTTPAPRPPTPAPTIASQPLSLRPEACRPAAGGAVHTRGLDFACD

SEQ ID NO: 11 - protein, 24 aa, Homo sapiens
  REGION 1..24: note = Transmembrane domain of CD8
IYIWAPLAGTCGVLLLSLVITLYC

SEQ ID NO: 12 - protein, 97 aa, Homo sapiens
  REGION 1..97: note = Intracellular signaling domain of CD3
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTAT

SEQ ID NO: 13 - protein, 42 aa, Homo sapiens
  REGION 1..42: note = Costimulatory signaling domain of CD137
KRGRKKLLYIFKQPFMRPVQTTQEEDGCSCRFPEEEEGGCEL

SEQ ID NO: 14 - protein, 454 aa, synthetic construct
  REGION 1..454: note = Full sequence of the EDA-binding CAR (without signal peptide)
EVQLLESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGSGGGGSEIVLTQSPGTLSLSPGERATLSCRASQSVSNAFLAWYQQKPGQAPRLLIYGASSRATGIPDRFSGSGSGTDFTLTISRLEPEDFAVYYCQQMRGRPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTAT

SEQ ID NO: 15 - protein, 21 aa, Homo sapiens
  REGION 1..21: note = CD8 Signal Peptide
MALPVTALLLPLALLLHAARP

SEQ ID NO: 16 - protein, 475 aa, synthetic construct
  REGION 1..475: note = Full sequence of EDA-binding CAR (with signal peptide)
MALPVTALLLPLALLLHAARPEVQLLESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGSGGGGSEIVLTQSPGTLSLSPGERATLSCRASQSVSNAFLAWYQQKPGQAPRLLIYGASSRATGIPDRFSGSGSGTDFTLTISRLEPEDFAVYYCQQMRGRPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTAT

SEQ ID NO: 17 - protein, 112 aa, Homo sapiens
  REGION 1..112: note = CD3z chain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 469 aa, synthetic construct
  REGION 1..469: note = human EDA CAR without signaling peptide complete CD3z chain
EVQLLESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGSGGGGSEIVLTQSPGTLSLSPGERATLSCRASQSVSNAFLAWYQQKPGQAPRLLIYGASSRATGIPDRFSGSGSGTDFTLTISRLEPEDFAVYYCQQMRGRPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 490 aa, synthetic construct
  REGION 1..490: note = human EDA CAR with signaling peptide complete CD3z chain
MALPVTALLLPLALLLHAARPEVQLLESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGSGGGGSEIVLTQSPGTLSLSPGERATLSCRASQSVSNAFLAWYQQKPGQAPRLLIYGASSRATGIPDRFSGSGSGTDFTLTISRLEPEDFAVYYCQQMRGRPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 1473 bp, synthetic construct
  source 1..1473: mol_type = other DNA
  misc_feature 1..1473: note = polynucleotide encoding the human EDA CAR of SEQ ID NO: 19
atggccctgcctgtgaccgccctgctgctgcccctggccctgctgctgcacgcggcgcgcccggaggttcagttgttggagagcggcggtggactcgttcagcctggtggttcattgcggctttcatgcgctgcaagtggattcaccttcagcgttatgaagatgtcttgggtccgacaagctccagggaagggacttgagtgggtgtccgccatctctggctcaggcggcagtacctattatgccgactcagtgaaggggagatttactatctctagagacaattctaagaataccctctatctccagatgaactcactgcgcgctgaagacaccgccgtgtattactgtgcaaaaagcacccatctctacctgtttgattactggggacaaggcacacttgtgacagtgagttctggcggcggtggttccgggggtggaggctccggaggtggaggtagcggcgggggtggttctgaaatagtactcactcaaagcccaggaaccctctctcttagcccaggtgagagggccactctctcctgtcgagcatctcagtccgtctctaacgctttcctggcatggtaccagcaaaaacccggccaagcaccacgacttctcatctacggtgctagtagcagagctaccggtatacccgaccgattctcaggttcaggaagtggaacagactttacattgaccatttcccgactggaacccgaggacttcgctgtatattactgccagcagatgcgcggacggccccccactttcggccaaggcactaaagtagagatcaagaccacaacccctgcccctaggccaccaacaccagcacctaccatcgctagccagccactgagcctgcggcccgaggcctgtaggccagccgccggcggagcagtgcacacccggggcctggactttgcctgcgatatctacatctgggcaccactggccggaacatgtggcgtgctgctgctgagcctggtcatcaccctgtattgcaagagaggcaggaagaagctgctgtacatcttcaagcagcccttcatgcggcccgtgcagacaacccaggaggaggacggctgcagctgtaggttccctgaggaggaggagggaggatgtgagctgcgcgtgaagttttctcggagcgccgatgcaccagcatatcagcagggacagaaccagctgtacaacgagctgaatctgggccggagagaggagtatgacgtgctggataagaggcgcggcagggaccctgagatgggcggcaagcctagaagaaagaacccccaggaaggcctgtataacgaactgcagaaagacaagatggccgaggcctacagcgagatcggaatgaagggcgagcggagaagaggcaagggccacgatggcctgtaccagggactgagcaccgccaccaaggatacctatgacgcactgcacatgcaggccctgccccccagatga

SEQ ID NO: 21 - protein, 505 aa, synthetic construct
  REGION 1..505: note = murine EDA CAR
MASPLTRFLSLNLLLLGESIILGSGEAEVQLLESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGSGGGGSEIVLTQSPGTLSLSPGERATLSCRASQSVSNAFLAWYQQKPGQAPRLLIYGASSRATGIPDRFSGSGSGTDFTLTISRLEPEDFAVYYCQQMRGRPPTFGQGTKVEIKQLTTTKPVLRTPSPVHPTGTSQPQRPEDCRPRGSVKGTGLDFACDIYIWAPLAGICVALLLSLIITLICYHSVLKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 22 - DNA, 1518 bp, synthetic construct
  source 1..1518: mol_type = other DNA
atggcaagtcctctgacaaggtttctctccctcaatctcctcctcctgggcgagtctatcattctgggtagcggtgaagccgaggttcagttgttggagagcggcggtggactcgttcagcctggtggttcattgcggctttcatgcgctgcaagtggattcaccttcagcgttatgaagatgtcttgggtccgacaagctccagggaagggacttgagtgggtgtccgccatctctggctcaggcggcagtacctattatgccgactcagtgaaggggagatttactatctctagagacaattctaagaataccctctatctccagatgaactcactgcgcgctgaagacaccgccgtgtattactgtgcaaaaagcacccatctctacctgtttgattactggggacaaggcacacttgtgacagtgagttctggcggcggtggttccgggggtggaggctccggaggtggaggtagcggcgggggtggttctgaaatagtactcactcaaagcccaggaaccctctctcttagcccaggtgagagggccactctctcctgtcgagcatctcagtccgtctctaacgctttcctggcatggtaccagcaaaaacccggccaagcaccacgacttctcatctacggtgctagtagcagagctaccggtatacccgaccgattctcaggttcaggaagtggaacagactttacattgaccatttcccgactggaacccgaggacttcgctgtatattactgccagcagatgcgcggacggccccccactttcggccaaggcactaaagtagagatcaagcaattgaccacaaccaagccagtgcttcgaaccccatcccctgtgcacccaacaggaacctctcagccacagaggcccgaggactgtaggccaagaggctccgtgaagggaacaggcctggactttgcctgtgatatctacatctgggctcccctggctggaatctgcgtggctctgctgctgagcctgatcatcaccctgatctgctaccactccgtgctgaagtggatcaggaagaagttcccccacatctttaagcagcctttcaagaagacaaccggagctgctcaggaggaggacgcttgctcttgtcgctgccctcaggaagaagaaggaggaggaggaggatacgagctgagggccaagttcagcagatccgctgagacagccgctaacctgcaggaccccaaccagctgtacaacgagctgaacctgggcaggagagaggagtacgacgtgctggagaagaagagggccagggaccccgagatgggaggaaagcagcagcggcgcaggaaccctcaggagggcgtgtacaacgctctgcagaaggacaagatggccgaggcttacagcgagatcggaaccaagggagagagacggcgcggaaagggacacgatggactgtaccagggcctgagcacagcaactaaagacacttacgatgccctccacatgcagaccctcgccccacggtga

SEQ ID NO: 23 - protein, 20 aa, synthetic construct
  REGION 1..20: note = scFv linker
GGGGSGGGGSGGGGSGGGGS

SEQ ID NO: 24 - DNA, 738 bp, synthetic construct
  source 1..738: mol_type = other DNA
  misc_feature 1..738: note = DNA sequence of the F8 ScFv clone
gaggttcagttgttggagagcggcggtggactcgttcagcctggtggttcattgcggctttcatgcgctgcaagtggattcaccttcagcgttatgaagatgtcttgggtccgacaagctccagggaagggacttgagtgggtgtccgccatctctggctcaggcggcagtacctattatgccgactcagtgaaggggagatttactatctctagagacaattctaagaataccctctatctccagatgaactcactgcgcgctgaagacaccgccgtgtattactgtgcaaaaagcacccatctctacctgtttgattactggggacaaggcacacttgtgacagtgagttctggcggcggtggttccgggggtggaggctccggaggtggaggtagcggcgggggtggttctgaaatagtactcactcaaagcccaggaaccctctctcttagcccaggtgagagggccactctctcctgtcgagcatctcagtccgtctctaacgctttcctggcatggtaccagcaaaaacccggccaagcaccacgacttctcatctacggtgctagtagcagagctaccggtatacccgaccgattctcaggttcaggaagtggaacagactttacattgaccatttcccgactggaacccgaggacttcgctgtatattactgccagcagatgcgcggacggccccccactttcggccaaggcactaaagtagagatcaag

SEQ ID NO: 25 - DNA, 750 bp, synthetic construct
  source 1..750: mol_type = other DNA
  misc_feature 1..750: note = DNA sequence of the C27 ScFv clone
gacgtacagctacaggaaagcgggcctgacctggtgaagccgtcccagtcattgtcactcacctgtacagtgacgggttactctatcacaagcgggtatagttggcactggatacgtcagttccccggaaataagcttgagtggatgggctacatccattattcaggcatcgctaattataacccctccttaaaatccaggatctcaattaccagagatacttctaaaaatcacttttttctccaactgaacagtgtcactacagaggataccgctacttactattgcgccaccgagaagactggctttttcgattactggggacagggtacaacacttaccgtgagctccggcggaggaggatccggcggtggcggttctggagggggaggcagcggggggggcggaagcgacgttgtgatgacgcaaacacctctgagtctgccagtcagtttgggcgaccaagccagcatcagctgccggagctcccagtctctggtgcactcgaacggtaacacctacttgcattggtacctgcagaagccagggcagtccccaaaactgctcatttacaaggtgtcaaacagattcagcggggtacctgaccgcttctctggctctggttccggtactgattttactctgaagatttcgcgagtcgaagcggaggaccttggagtttatttctgtagtcagtctgcacacgtccctcccaccttcggcggcgggaccaaactcgaaataaagagg

SEQ ID NO: 26 - protein, 250 aa, synthetic construct
  REGION 1..250: note = Protein sequence of the C27 ScFv clone
DVQLQESGPDLVKPSQSLSLTCTVTGYSITSGYSWHWIRQFPGNKLEWMGYIHYSGIANYNPSLKSRISITRDTSKNHFFLQLNSVTTEDTATYYCATEKTGFFDYWGQGTTLTVSSGGGGSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSAHVPPTFGGGTKLEIKR

SEQ ID NO: 27 - DNA, 717 bp, synthetic construct
  source 1..717: mol_type = other DNA
  misc_feature 1..717: note = DNA sequence of the C33 ScFv clone
gaggtcaaactggtcgaatcagggggcggccttgtaaagcccggaggatctctgaaattgagctgtgctgccagcggtttcacattttccaacagcgccatgacttgggtgcgccagacacctgagaagaggctcgagtgggtggcgtcaatatccggcggaggtaccacctactatcctgactcagtgaagggaagattcacgatcagtagagataacgccaggaacatcctgtacttgcagatgtcatctctacgctccgaagacacagctatttattactgcgcccggtcgcactattggggacagggcaccactttaaccgtgagctctggtgggggaggttctggcggtggtggcagtggaggcggcggcagcggcggaggggggtcagatattgtcatgacgcaaagccagaagtttatgtccacatctatcggagaccgtgtttctgtgacatgtaaggcctctcaaaatgttgtgactaacgtagcatggtaccagcagaaaccagggcagtccccaaaagcactgatctacagtgcatcctataggtattcgggcgtccccgatcgattcactgggagcggcagtgggactgactttacacttaccatctccaatgtgcagagcgaggatctcgctgagtacttctgccagcaatataatagttacccgtacacctttggtgggggaaccaagctggaaattaagcgg

SEQ ID NO: 28 - protein, 239 aa, synthetic construct
  REGION 1..239: note = Protein sequence of the C33 ScFv clone
EVKLVESGGGLVKPGGSLKLSCAASGFTFSNSAMTWVRQTPEKRLEWVASISGGGTTYYPDSVKGRFTISRDNARNILYLQMSSLRSEDTAIYYCARSHYWGQGTTLTVSSGGGGSGGGGSGGGGSGGGGSDIVMTQSQKFMSTSIGDRVSVTCKASQNVVTNVAWYQQKPGQSPKALIYSASYRYSGVPDRFTGSGSGTDFTLTISNVQSEDLAEYFCQQYNSYPYTFGGGTKLEIKR

SEQ ID NO: 29 - protein, 90 aa, Mus musculus
  REGION 1..90: note = murine EDA protein sequence
NIDRPKGLAFTDVDVDSIKIAWESPQGQVSRYRVTYSSPEDGIRELFPAPDGEDDTAELQGLRPGSEYTVSVVALHDDMESQPLIGIQST

SEQ ID NO: 30 - protein, 90 aa, Homo sapiens
  REGION 1..90: note = human EDA protein sequence
NIDRPKGLAFTDVDVDSIKIAWESPQGQVSRYRVTYSSPEDGIHELFPAPDGEEDTAELQGLRPGSEYTVSVVALHDDMESQPLIGTQST

(19) United States
(12) Patent Application Publication
(10) Pub. No.: US 2026/0007697 A1
(43) Pub. Date: Jan. 8, 2026

(54) NEW CHIMERIC ANTIGEN RECEPTOR (CAR) CELLS AND MEDICAL USES THEREOF

(71) Applicant: FUNDACIÓN PARA LA INVESTIGACIÓN MÉDICA APLICADA, Pamplona (ES)

(72) Inventors: Juan José LASARTE SAGASTIBELZA, Pamplona (ES); Teresa LOZANO MOREDA, Pamplona (ES); Celia MARTÍN OTAL, Pamplona (ES)

(21) Appl. No.: 18/844,776

(22) PCT Filed: Mar. 8, 2023
(86) PCT No.: PCT/EP2023/055924

§ 371 Date: Sep. 6, 2024

(30) Foreign Application Priority Data
Mar. 8, 2022 (EP) 22382215.6
Jul. 14, 2022 (EP) 22382672.8

(51) Int. Cl. A61K 35/17; A61K 40/11; A61K 40/15; A61K 40/31; A61P 35/00; C07K 14/705; C07K 14/725; C07K 16/18; C12N 15/86

(52) U.S. Cl. CPC A61K 35/17; A61K 40/11; A61K 40/15; A61K 40/31; A61P 35/00; C07K 14/7051; C07K 14/70517; C07K 14/70578; C07K 16/18; C12N 15/86; A61K 2239/17; A61K 2239/21; A61K 2239/22; C07K 2317/565; C07K 2317/622; C12N 2740/13043; C12N 2740/15043

(57) ABSTRACT

The present invention relates to a new Chimeric Antigen Receptor (CAR) targeting the extra-domain A of fibronectin (EDA), to nucleic acid sequences and vectors encoding thereof, and host cells expressing the same. It further relates to methods of producing thereof, pharmaceutical compositions, kits, methods of treatment, and combination therapies using thereof.

### FIELD OF THE INVENTION

The present invention relates to the field of biomedicine and immunotherapy. Specifically, it relates to a new Chimeric Antigen Receptor (CAR) targeting the extra-domain A of fibronectin (EDA), to nucleic acid sequences and vectors encoding thereof, and host cells expressing the same. It further relates to methods of producing thereof, pharmaceutical compositions, kits, methods of treatment, and combination therapies using thereof.

### STATE OF THE ART

Chimeric Antigen Receptor T-cell (CAR-T) therapy has shown encouraging antitumor efficacy in some hematological tumors (Brentjens R J et al., 2013; Grupp S A et al., 2013) and constitutes a very promising advanced therapy for the treatment of cancer. However, the initial response rates achieved with the current generation of anti-CD19 CART cells in certain malignant B-cell tumors do not apply equally to other hematological cancers and much less to solid tumors, where many challenges still need to be overcome (Wagner J et al., 2020).

On the one hand, it is extremely difficult to find a specific antigen expressed in the cell membrane for each type of tumor. Although most of the current CAR-T cell therapies target the tumor directly (Hartmann J et al, 2017), solid tumors rarely display unique antigenic markers and the engagement of target antigen on non-tumor tissues entails the risk of “on-target/off-tumor” toxicity (Lamers C H et al., 2016; Parkhurst M R et al., 2011). On the other hand, solid cancers have some formidable barriers to the action of antitumor T lymphocytes (Wagner J et al., 2020). The complexity of the extracellular matrix (ECM) of tumors with excessive intratumoral deposition of collagen, fibronectins, laminins or hyaluronan (Caruana I et al., 2015) and the lack of chemokines capable of attracting T cells has been reported to lead to an inefficient homing and tumor penetration of redirected T cells (Jin L et al, 2019).

It is accepted that carcinomas behave like wounds, which force the tumor microenvironment (TME) into a constant state of fibrotic repair (Dvorak H F et al., 1986) with a continuous and extensive remodeling of ECM. This highly fibrotic structure, with the accumulation of extensively crosslinked type I collagen fibrils (Perryman L et al., 2014), significantly affects tumor progression, metastasis and response-to-therapy being a biomarker of poor outcome (Jiang H et al., 2017; Keely P J et al., 2011; Werb Z et al., 2015 Paszek M J et al., 2005). Thus, the aberrant vasculature and the fibrotic state of desmoplastic tumors can act as a mechanical barrier that impairs lymphocyte tumor infiltration (Bergers G et al., 2003; Itatani Y et al., 2018) and there is a need to find new CAR cell therapies that are useful for the treatment of the so-called “cold” or “immune excluded” tumors where antitumor lymphocytes do not access the tumor bed (Salerno E P et al., 2016).

As mentioned above, during cancer progression, the ECM undergoes dramatic changes which promote cancer cell migration and invasion. Tumor ECM components not commonly found in healthy tissues have been proposed as therapeutic targets for CAR-T cells. For instance, T cells expressing CARs targeting tumor vasculature antigen VEGF-1 or VEGF-2 demonstrated a significant delay of tumor growth in pre-clinical experiments (Chinnasamy D et al., 2010; Wang W et al., 2013). However, tumor endothelial cells exhibit a remarkable heterogeneity, hence targeting one antigen might not be enough to achieve the desired antiangiogenic effect (Patten S G et al., 2010). Other CAR-T cells attempt to disrupt tumor neovasculature targeting αvβ3 integrin (Fu X et al., 2013), TEM8 (Byrd T T et al., 2018; Petrovic K et al., 2019) or CLEC14A (Zhuang X et al., 2020), although some toxicity issues have also been evidenced (Chinnasamy D et al., 2010; Petrovic K et al., 2019) probably because of the “on-target off-tumor” activity of the CAR-T cells.

In the remodeled tumor ECM, fibronectin acts as a central organizer of ECM molecules and mediates the crosstalk between the tumor microenvironment and cancer cells (Efthymiou G et al, 2020). Fibronectin (FN) is a high-molecular weight (˜500-˜600 kDa) glycoprotein of the ECM that binds to membrane-spanning receptor proteins called integrins. FN also binds to other extracellular matrix proteins such as collagen, fibrin, and heparan sulfate proteoglycans. Although FN is encoded by a single gene, the alternative splicing of its pre-mRNA and the incorporation of extra domains result in the formation of cell- and tissue-specific FN isoforms. Splicing occurs at three sites, with the possibility of incorporating the extra domain A (EDA), extra domain B (EDB), or the IIICS domain. Fibronectin isoforms comprising the EDA or EDB domains are also known as oncofetal forms because they are expressed during embryonic development, restricted in normal adult tissues and re-expressed in adults during cancer progression (Wang J P et al, 2017). These spliced versions of FN present in the ECM belong to cellular fibronectin type and are not present in soluble plasma fibronectin.

EDA has been reported to be strongly expressed in many tumor types as compared to normal tissues (reviewed in Kumra H et al., 2016), and this increased expression has been correlated with cancer progression, dissemination and poor prognosis. Besides, antibody-cytokine or antibody-enzyme conjugates based on EDA-specific monoclonal antibodies have shown antitumor activity in preclinical animal models (Moschetta M, et al., 2012; Rosini E, et al., 2021). Both EDA and EDB have been shown to be highly expressed in several tumors and tumor neovasculature (Efthymiou G et al., 2020) suggesting a role in tumor progression. However, it is becoming clear that the alternatively spliced EDA and EDB domains may exert different functions (Moschetta M. et al., 2012). Recently a strategy has been proposed by Wagner et al. using CAR-T cells against the extra domain B of fibronectin, showing antitumor and antivascular activity in preclinical mouse tumor models (Wagner J et al., 2021; Xie Y J et al., 2019). However, the authors detected some transient expansion of the EDB-targeted CAR-T cells in non-tumor bearing mice, and, although not signs of toxicity were observed in their study, the results suggest a potential CAR-T cells stimulation by low EDB expression in normal tissues.

Accordingly, there is still the need of identifying target antigens for CAR cell-based therapies for the effective and safe treatment of solid tumors, specially to identify target antigens which enable avoiding “on-target/off-tumor” toxicity which occurs through engagement of the target antigen by the CAR-T cells on non-tumor tissues.

### SUMMARY OF THE INVENTION

The present invention provides a new CAR cell therapy strategy that allows the treatment of diverse types of cancer, in particular solid tumors originating in different organs and tissues, using CAR cells (e.g. CAR-T cells) against a single antigen, namely against the extra domain A (EDA) of fibronectin.

From the TCGA SpliceSeq analysis as well as from the RNAsec analysis of human hepatocarcinoma (HCC) samples, the inventors have confirmed the upregulation of the EDA in several tumor types. The immunohistochemical analysis in biopsies from different tumor types, confirmed the positive staining for EDA in tumor stroma and vessels from cholangiocarcinoma, hepatocarcinoma, pancreas and in ovarian carcinoma (Example 1), expanding the previous results on the strong EDA staining of vascular structures in a panel of human tumors (Rybak J N et al., 2007; Glukhova M A et al., 1990) and supporting the potential use of EDA as a tumor target antigen.

The inventors generated EDA-specific CAR-T cells that were tested in vitro (Example 3) and in vivo for their capacity to reject tumors (Examples 4 and 5). More specifically, the experimental data provided herein demonstrated that EDA CAR-T cells recognize EDA in an antigen-dependent manner and display antitumor activity in vivo in several murine tumor models, such as immunocompetent C57BL/6J mice challenged with PM299L hepatocarcinoma cell line, expressing medium (Example 4.1) and high (Example 4.2) levels of EDA, immunocompetent 129Sv mice bearing F9 teratocarcinoma expressing EDA in the tumor endothelium (Example 4.3) or NSG mice challenged with the human hepatocarcinoma cell line PLC (Example 6).

Indeed, the observed strong antitumoral effect in the NSG xenograft model of human hepatocarcinoma cell line PLC, which expresses EDA in the tumor stroma and the endothelial vasculature, for the generated human EDA CAR-T cells, including the human 41BB and CD3ζ endodomains, suggests a potential translation to human settings for the treatment of human tumors with upregulated EDA expression.

Inmunocompetent mice were challenged with an EDA-expressing established tumor. Seven days later, when tumors were palpable, the mice were treated with a mixture of CD4⁺ and CD8⁺ EDA CAR-T or PSMA CAR-T cells. All mice treated with EDA CAR-T, but not with PSMA CAR-T cells, rejected the tumor. Moreover, it was found that 4 days after T cell therapy, mice treated with EDA CAR-T showed a significantly higher number of intratumoral CAR-T cells, with a higher level of activation (measured as the percentage of CD4⁺ or CD8⁺ CD137⁺ cells), but also a higher percentage of PD1⁺ or TIGIT⁺ T cells than those treated with PSMA CAR-T (Example 4.2). Thus, the EDA CAR-T cells showed a good penetration of the tumor and specific activation when encountering the antigen in the tumor.

Moreover, the inventors did not observe any clinical signs of toxicity in the EDA CAR-T cell treated animals suggesting that EDA is not expressed in healthy tissues, at least at the level needed for CAR-T cell activation, and also that EDA CAR-T cells are well-tolerated with no noticeable “on-target/off-tumor” toxicity (Example 5).

Without willing to be bound by theory, EDA CAR-T cells may directly exert its antitumor effects by recognizing EDA in the extracellular matrix and be activated to produce cytokines such as IFN-γ, that can exert strong antiangiogenic effects (FIG. 8E), reduce fibrotic markers, such as α-SMA (FIG. 8F), and impair tumor growth (FIGS. 8A & 8G). Transcriptomic analysis of tumor samples was done to evaluate the impact of EDA CAR T therapy in the tumor microenvironment. EDA CAR T treatment was found by the inventors to exert an antiangiogenic effect and have a profound effect in remodeling the tumor microenvironment by significantly reducing gene signatures associated with epithelial-mesenchymal transition, collagen synthesis, extracellular matrix organization as well as IL-6-STAT5 and KRAS pathways (FIG. 10). The reduction in collagen synthesis and extracellular matrix organization gene signatures suggests that treatment with the EDA CAR-T cells results in a less fibrotic stroma which is thus more easily accessible to immune cells.

Interestingly, EDA CAR-T cell infusion showed antitumor therapeutic efficacy against the challenge with tumor cells not expressing EDA (Example 4.3). In the F9-cell based tumor model, the inventors have found that EDA is located in the basement membrane of the endothelium and not in the tumor cells (see co-localization experiments, FIG. 1H). This observation may be explained by the deposition of EDA-containing fibronectin fibers in the basal membrane of the endothelium. Without willing to be bound by theory, it is plausible that the observed tumor inhibitory effect in EDA-negative tumors is the result of an indirect mechanism wherein soluble EDA is bound to the surface of endothelial or tumoral cells expressing EDA receptors, such as integrins or TLR4, thus these cells becoming a target for the EDA-CART cells of the invention. This indirect mechanism is supported by the observed activation of triple parameter reporter (TPR) Jurkat cells expressing the EDA CAR by EDA-coated Jurkat wild type cells (FIG. 12, Panel B).

Moreover, a comparative assay (Example 8) was conducted between the F8 clone and two other scFv clones against EDA (clones C27, C33) generated by the inventors using the VH and VL domains of antibodies 27A12.70 and 33E3.10 described in WO2015/088348 A1. Retroviruses encoding CARs comprising these anti-EDA scFv were prepared and used to generate corresponding EDA CAR-T cells. We tested the ability of these CARTs to recognize the recombinant EDA protein of murine and human origin and to produce interferon-gamma. It was observed that both the C33 and F8 clones recognize both proteins while the C27 clone only recognizes the EDA protein of human origin. We have observed that clone C33 shows a higher in vitro affinity for the EDA protein than clone F8 (FIGS. 14 and 15). However, this greater affinity of the C33 did not translate into a greater in vitro recognition of tumor cells that express EDA. Conversely, the EDA CART carrying the ScFv clone F8 recognized very efficiently these PM299L tumor cells that express EDA on their surface (FIG. 16). This better recognition capacity of the EDA CART carrying the ScFv clone F8 was found to be accompanied by a greater antitumor efficacy in the F9 model of teratocarcinoma. It was found though, that both EDA CARTs showed significant antitumor efficacy in this model (FIG. 17). Accordingly, we confirmed the anti-tumor effect of the anti-EDA CAR T-cells using different scFv clones as EDA-targeting moiety of the CAR.

Therefore, in accordance with one aspect of the invention, the invention relates to a chimeric antigen receptor (CAR)—also referred herein a chimeric receptor—nucleic acid comprising a polynucleotide coding for a ligand binding domain comprising an extra-domain A of fibronectin (EDA) targeting-moiety, optionally, a polynucleotide coding for a hinge or spacer region, a polynucleotide coding for a transmembrane domain; and a polynucleotide coding for an intracellular signaling domain. In preferred embodiments, the CAR nucleic acid further comprises a polynucleotide encoding a costimulatory signaling domain. In other preferred embodiments, the coding polynucleotide of the EDA-targeting moiety is operably linked to a hinge or spacer region coding polynucleotide which is operably linked to a transmembrane region coding polynucleotide which is operably linked to a polynucleotide coding for a costimulatory signaling domain which is operably linked to a intracellular signaling domain coding polynucleotide.

In a second aspect the invention refers to a chimeric antigen receptor (CAR) polypeptide—also referred herein as a chimeric receptor polypeptide-coded for by a chimeric receptor nucleic acid as described herein.

In a third aspect the invention refers to an expression vector comprising a chimeric receptor nucleic acid as described herein.

In a fourth aspect, the present invention provides a cell comprising the nucleic acid, a vector and/or the CAR polypeptide as described herein.

In a fifth aspect, the present invention provides a pharmaceutical composition comprising a host cell, such as a plurality of cells, of the present invention and a pharmaceutically acceptable excipient, carrier or diluent.

In a sixth aspect, the present invention provides a chimeric receptor nucleic acid comprising:
a) a polynucleotide coding for a ligand binding domain comprising an extra-domain A of fibronectin (EDA) targeting-moiety as described herein, preferably a single chain Fv (scFv);
b) optionally, a polynucleotide coding for a hinge or spacer region;
c) a polynucleotide coding for a transmembrane domain as described herein; and
d) a polynucleotide coding for an intracellular signaling domain as described herein;
   or a chimeric receptor polypeptide coded for thereby as described herein;
   an expression vector comprising the chimeric receptor nucleic acid as described herein;
   a host cell comprising the chimeric receptor nucleic acid or the expression vector as described herein; or
   a pharmaceutical composition comprising any thereof and further comprising and a pharmaceutically acceptable excipient, diluent or carrier as described herein for use as a medicament.

In a seventh aspect, the present invention provides a chimeric receptor nucleic acid comprising:
a) a polynucleotide coding for a ligand binding domain comprising an extra-domain A of fibronectin (EDA) targeting-moiety as described herein, preferably a single chain Fv (scFv);
b) optionally, a polynucleotide coding for a hinge or spacer region;
c) a polynucleotide coding for a transmembrane domain; and
d) a polynucleotide coding for an intracellular signaling domain; or
   a chimeric receptor polypeptide coded for thereby as described herein;
   an expression vector comprising the chimeric receptor nucleic acid as described herein;
   a host cell comprising the chimeric receptor nucleic acid or the expression vector as described herein; or
   a pharmaceutical composition comprising any thereof and further comprising and a pharmaceutically acceptable excipient, diluent or carrier as described herein for use in a method of treating an EDA-positive cancer.

In a related aspect the present invention refers to a method for the treatment of an EDA-positive cancer in a subject, said method comprising administering the polynucleotide coding for a CAR as described herein, a CAR polypeptide as described herein, a vector as described herein, a host cell as described herein or a pharmaceutical composition as described herein to a subject in need thereof.

In a further related aspect, the invention provides the use of the polynucleotide coding for a CAR as described herein, a CAR polypeptide as described herein, a vector as described herein, a host cell as described herein or a pharmaceutical composition as described herein in the manufacturing of a medicament for the treatment an EDA-positive cancer in a subject in need thereof.

In a further aspect, the invention relates to CAR Tregs of the invention for use in the treatment or prevention of diseases mediated by an excessive or inadequate response of the immune system, such as transplant rejection, especially cardiac transplant rejection. It further pertains to related methods of treatment.

The invention further provides a kit which comprises a dosage form of a CAR nucleic acid, the encoded CAR polypeptide, a vector, a host cell or a pharmaceutical composition as described herein, optionally with a dosage form of another drug; and instructions for the use thereof.

### BRIEF DESCRIPTION OF THE FIGURES

FIG. 1. EDA expression on tumor tissues (A, B) Percentage of spliced fibronectin in several types of human tumor samples compared with healthy tissue. RNAseq analysis from TCGASpliceSeq data set (A.1), and from RNAseq analysis from TCGASpliceSeq data set having paired (tumor and non-tumor) data (A.2) or from a cohort of patients with hepatocarcinoma (B.1 and B.2). EDA expression in tumor samples (black bars) and in healthy tissue (grey bars) is plotted. (C, E) Immunohistochemical detection of EDA in tumor biopsies from different cancer patients and the corresponding healthy tissue as control (C) and from tumor biopsies from NSG mice xenografted with human hepatocarcinoma (PLC, HUH7) or cholangiocarcinoma (HUCCT and TFK1) cell lines (E). (D, F) Quantification of EDA expression in the indicated tumor samples and healthy tissues measured by using ImageJ software (G) Confocal Immunofluorescence analysis of the expression of CD31 and EDA in PLC, HUH7, HUCCT and TFK1 tumor samples obtained from NSG mice). (H) Co-localization experiments of EDA and type IV collagen in F9 and PLC tumors as well as normal kidney. Paraffin-embedded tissue sections were labeled with anti-Collagen-IV, EDA antibodies and DAPI for nuclear counterstain and studied by confocal microscopy. (I) EDA expression in tumor samples from NSG mice challenged with human hepatocarcinoma and cholangiocarcinoma cell lines. The anti-human EDA IST-9 antibody and the scFv F8 was used to detect the murine and the human versions of EDA protein. Human biopsies from patients with hepatocarcinoma or cholangiocarcinoma were used as positive controls for EDA staining with each antibody. *p<0.05, ****p<0.001. Student t test. Bars representing the mean and SD are plotted. T: Tumor. N: Normal. PSI: Percent-spliced-in value

FIG. 2. Flow cytometry analysis of EDA expression in different tumor murine cell lines and tumors. (A) B16-OVA melanoma, LLC-OVA lung carcinoma and F9 teratocarcinoma were cultured in vitro in the absence or presence of TGFβ for 16 h and stained for the analysis of EDA expression by flow cytometry. Representative cases of the flow cytometry analysis (upper panel) and histograms summarizing the results obtained (lower panel). (B) EDA expression in tumor tissues induced in mice after subcutaneous injection of the indicated cell lines at day 15 after tumor challenge. Representative examples of the immunohistochemical analysis. (C) EDA/CD31 co-localization experiments in B16-OVA and F9 derived tumor sections at day 15 after tumor challenge.

FIG. 3. Generation and in vitro characterization of EDA CAR-T cells. (A) Scheme representing the second generation EDA CAR and control PSMA CAR structures. (B) CAR expression measured on the surface of transduced T cell, both CD8 (right) and CD4 (left) (C) EDA ligand binding assay for EDA CART (gray dark histogram) or control PSMA CAR-T (gray light histogram). CD4+ or CD8+ CAR-T cells were incubated with recombinant human and mouse His-EDA protein (50 μg/ml) and stained with anti His-APC antibodies. Numbers in histograms indicate mean fluorescence intensity. (D-E) CAR-T function after antigen-specific stimulation. (D) IFN-γ production by EDA CAR-T or PSMA CAR-T cells after 48 hours of stimulation with mouse EDA (m) or human EDA (h) protein coated plates. (E) CD4+ or CD8+ CAR-T cell proliferation in response to EDA measured by 3H-Timidine incorporation. Data are representative of three independently repeated experiments. **p<0.01, ***p<0.005, ****p<0.001. One-way ANOVA with Bonferroni multiple comparisons test (D, E). Bars representing the mean and SD are plotted.

FIG. 4. EDA CAR-T cells recognize EDA-expressing tumors. (A-B) Number of IFN-γ producing cells in response to PM299L-EDA C2, C3 and C7 clones. CD4 (A) and CD8 (B) CAR-T cell were stimulated for 48 h with PM299L expressing high levels of EDA (clones C2 and C7) or low levels of EDA (clone C3) on their membrane or with wild type PM299L-Thy1.1 cell lines at 1:1 ratio. (C) CAR-T cell proliferation, CD69 expression and IL-2 or IFN-γ production in response to stimulation with PM299L-EDA C7 (EDA-high) or with PM299L-Thy1.1. (D) Lytic activity of CAR-T cells. (Left panel) Lysis of PM299L-EDA^{high} (clone C7) or PM299L-EDA^{low} (clone C3) tumor target cells measured by flow cytometry after 24 h of cell cocultures (1:1 or 0,2:1 CAR-T: target cell ratio). (E) Real-time cytotoxicity measured by xCELLigence: percentage of specific lysis was analyzed after 4 hours of co-culture. *p<0.05, **p<0.01, ****p<0.001. One-way ANOVA with Bonferroni multiple comparisons test (A, B). Bars representing the mean and SD are plotted. NT: no stimulated. Clones PM299L-EDA C2 and C7 express high levels of EDA. PM299L-EDA C3: express low levels of EDA. PM299L-Thy.1.1: EDA negative cell line used as a control.

FIG. 5. Generation and characterization of EDA expressing PM299L tumor cell clones. (A) Schematic representation of the procedure for clone selection. (B) Flow cytometry analysis of EDA expression levels of different PM299L EDA expressing clones. Numbers in histograms indicate mean fluorescence intensity. (C) Immunohistochemical analysis of EDA expression of tumor tissues obtained from C57BL/6 mice challenged with the indicated PM299L EDA expressing clones.

FIG. 6. Antitumor activity of EDA CAR-T therapy in EDA+ tumors. (A) C57BL/6 J mice were injected subcutaneously with 2×10⁶ cells PM299L-EDA^{high} tumor cells. Four h later, mice were treated with EDA-CART. At day 40, mice were rechallenged with PM299L-EDA^{high} tumor cells. Mean tumor are is ploted (left panel). EDA-CART cell expansion measured in blood of mice after tumor re-challenge (right panel). (B) C57BL/6 J mice were injected subcutaneously with 2×10⁶ cells PM299L-EDA C7 tumor cells. At day 7, mice bearing tumors were treated by adoptive transfer with 9×10⁶ EDA CAR-T or with PSMA CAR-T cells as a control. Tumor growth and Kaplan Meier survival curves are represented (n=7 mice per group). Data are representative of two independently repeated experiments. (C) Percentage of tumor infiltrating CAR-T cells and expression of CD137, PD1 and TIGIT in PSMA CAR-T and EDA CAR-T cells. (D) Percentage of proliferating (Ki67⁺ IFN-γ⁺) CD4⁺ and CD8⁺ EDA CAR-T and PSMA CAR-T cells isolated from the spleen of mice bearing tumors in response to EDA stimulation. (n=4 mice per group). (E) Migratory capacity of the infused CAR-T to the site of antigen expression. Mice bearing the PM299L-EDA C2 in the right flank and the original PM299L tumor cell line (EDA negative) in the left flank were treated intravenously with EDA CAR-T or PSMA CAR-T cells and seven days later, the CAR-T cells infiltrating each tumor were analyzed by flow cytometry. Data are representative of two independently repeated experiments. *p<0.05, **p<0.01, ***p<0,005, ****p<0.001. Survival curve were analysed by Logrank test (A, C). Student's t-test (D), One-way ANOVA with the Bonferroni multiple comparisons test (E). Bars representing the mean and SD are plotted. Each symbol represents an individual mouse.

FIG. 7. Lack of toxicity after EDA CAR-T cell administration in mice. Mice bearing PM299L tumors were treated with 1×10⁷ CAR-T cells (5×10⁶ CD8 and 5×10⁶ CD4). At day 7 after CAR-T cell infusion blood and organs (liver, spleen, lung, kidney and heart) were collected from the mice and stained with hematoxylin and eosin (H&E) for toxicity evaluation. (A.1) Body weight was measured and serum samples were analyzed for measure biochemical parameters; AST: aspartate aminotransferase, ALT: alanine aminotransferase, serum albumin, AMYL2: alpha Amylase, Urea, Creatinine, CRPLX: C-reactive protein, ALP: alkaline phosphatase, LDH: lactate dehydrogenase; (B.1) H&E staining of liver, spleen, kidney, lung and heart tissue sections (scale bar 200 μm; right, scale bar 50 μm); Naïve C57BL6 J mice were treated with 1×10⁷ CAR-T cells (5×10⁶ CD8 and 5×10⁶ CD4). (A.2) Body weight was measured periodically during all the follow up and serum samples were analyzed at day 17 and day 30 to measure biochemical parameters; AST: aspartate aminotransferase, ALT: alanine aminotransferase, serum albumin, AMYL2: alpha Amylase, Urea, Creatinine, CRPLX: C-reactive protein, ALP: alkaline phosphatase, LDH: lactate dehydrogenase (B.2) H&E staining of thymus, liver, hearth, lung, and kidney tissue sections obtained at day 30 of the follow up. (scale bar 200 μm; right, scale bar 50 μm). (C) EDA expression detected in human samples from a healthy liver, from 3 patients with alcoholic cirrhosis, 2 patients with HCV and 2 HBV related cirrhosis, 4 primary biliary cirrhosis, 2 autoimmune and 1 cryptogenic cirrhosis as compared to that observed in patients with hepatocarcinoma (HCC). (D) EDA expression in an animal model of liver fibrosis induced by CCL4 treatment and a model of inflammatory colitis induced by DSS administration. The corresponding tissue sections from untreated healthy mice were used as controls. (E) EDA expression detected in human samples from a healthy liver, from 3 patients with alcoholic cirrhosis, 2 patients with HCV and 2 HBV related cirrhosis, 4 primary biliary cirrhosis, 2 autoimmune and 1 cryptogenic cirrhosis as compared to that observed in patients with hepatocarcinoma (HCC).

FIG. 8. EDA CAR-T cells delay F9 teratocarcinoma tumor growth. (A) 129Sv mice bearing F9 tumor cells (n=10 mice per group) were treated with a single dose of 1×10⁷ of EDA CAR-T or PSMA CAR-T cells. Tumor growth at different time points (left) and Kaplan-Meier plot of survival of mice bearing tumors (right) are plotted. (B) Characterization of PD1+ CAR-T cells infiltrating the tumor 7 days after cell transfer (n=5). (C, D) Percentage of IFN-γ producing CD4 or CD8 CAR-T cells in the draining lymph nodes (C) and in the spleen (D). Functional analysis after short PMA/ionomycin stimulation on CD4 CAR-T cells 7 days after adoptive T cell transfer (left) and on endogenous CD8 (right) in draining lymph nodes. (D) Splenocytes were stimulated with PM299L cells (10 splenocytes: 1 tumor cell), after 16 hours, the number of IFN-γ producing cells was measured by ELISPOT. (E) Direct in vivo estimation of tumor vasculature using Angiosense 750 at days −1 and day 14 after EDA CAR-T-cell infusion, expressed as relative fluorescence units (RFU) divided by tumor volume in SV129 mice bearing F9 subcutaneous tumors. Untreated mice were used as negative control (n=5-6 mice/group). (F) α-SMA immunohistochemistry analysis on tumor samples at day 14 after EDA CAR-T therapy. Two representative images for untreated and EDA-CART treated mice and the quantification of α-SMA positive area are shown. Data are representative of two independently repeated experiments. (G) Effect of IFN-γ neutralization on the antitumor effect of EDA-CART cell infusion. (A) Survival curve was analyzed by the log-rank test. Student t test (B, C-left), One-way ANOVA with Bonferroni multiple comparisons test (C-right, D). Paired-t test (E). The mean and SD for each group are plotted. Each symbol represents an individual mouse. ns=non-significant, *p<0.05, **p<0.01, ****p<0.001.

FIG. 9. Flow cytometric analysis of leukocyte infiltration into the F9 tumors treated with EDA-CART, PSMA-CART or treated only with TBI+IL2. (A) Percentage of CD4⁺ CAR⁻, CD8⁺ CAR⁻, CD11b, CD11c, NKp46 or F4/80⁺ cells in total CD45^{+ve} cells. (B) Percentage of Ly6G⁺ Ly6C⁻ and Ly6G⁺ Ly6C⁺ in total CD11b⁺ cells. (C) Percentage of MHCII^{high} (M2) and CD206 (M2) cells in total F4/80⁺ cells. (D) Percentage of CD25+ Foxp3+ cells in total CD4⁺ cells and % ICOS+ TIGIT+ cells in total Treg cells. (E) Lack of efficacy of EDA CAR-T cells in mice bearing B16OVA tumor cells. C57BL/6 mice bearing tumors were treated 7 days after tumor challenge with CD8+ or a mixture of CD4+ and CD8+ EDA CAR-T or PSMA CAR-T lymphocytes. (left) Mean tumor growth at different time points and (right) Kaplan-Meier plot of survival are plotted. (n=6-8 mice per group).

FIG. 10. Transcriptomic analysis of tumor tissues isolated from untreated mice and from mice treated with EDA CART cells. 129Sv mice bearing F9 tumor cells were treated with EDA CAR-T or left untreated and 14 days after adoptive cell transfer, mRNA from tumor samples was isolated for RNASec analysis. (A) Volcano plots for differential gene expression of Control vs EDA-CART cell treated mice. Genes with a padj<0.05 were considered differentially expressed. (B) Hallmark gene set enrichment analysis with positive and negative enrichment scores for specific biological states in untreated and EDA-CART treated mice. (C) Selected gene sets with a significant negative enrichment in tumors treated with EDA-CART cells. FDR q values were calculated using GSEA software.

FIG. 11. EDA CAR-T cells have antitumor activity in NSG mice xenotransplanted with the human hepatocarcinoma cell line PLC. (A) Human EDA CAR binding to EDA protein measured by flow cytometry. Numbers in histogram indicate mean fluorescence intensity. (B) Flow cytometric analysis of the activation of NFAT, AP1 and NF-kB in the triple parameter T cell reporter Jurkat cell line expressing the human EDA CAR in response to different doses of the EDA protein. (C) Cytokine production and proliferation of human EDA CAR-T or a control CAR-T cells in response to EDA stimulation. (D) Antitumor activity in NSG mice xenotransplanted with the human hepatocarcinoma cell line PLC. NSG mice (n=6-9 mice per group) were challenged with PLC tumor cells and, 8 days later, when the tumors reached 5 mm in diameter, mice were treated with 5×10⁶ untransduced or EDA CAR transduced T cells. (D) Mean tumor area at different time points (left) and tumor area of each individual mouse in each experimental group (right). (E) Phenotypic analysis of CD4 or CD8 EDA CAR-T cells isolated from the spleen or tumors, seven days after adoptive transfer. Activation markers (in green) and exhaustion markers (in red) were analyzed by flow cytometry. Data are representative of two independently repeated experiments. Two-way ANOVA with the Bonferroni multiple comparisons test ****p<0.001. The mean and SD for each condition are plotted.

FIG. 12. Flow cytometry analysis of the activation of NFAT, AP1 and NF-kB in the triple parameter reporter Jurkat TPR cell line expressing the EDA-CAR in response to stimulation with wild type Jurkat cells previously incubated with EDA protein. (A) Measurement of EDA binding to wild type Jurkat cells. Cells were incubated with 25 μg/ml of EDA protein for 30 min at 37° C., washed and labeled with anti-His-APC labelled antibodies. EDA binding to Jurkat cells was then measured by flow cytometry. (B) Activation of NFAT, AP1 or NF-kB in Jurkat TPR reporter cell line in response to their co-culture with wild type Jurkat cells previously incubated with EDA. ****p<0.0001. Two-way ANOVA with Bonferroni multiple comparisons test. NL: non labelled.

FIG. 13. Production and characterization of the anti EDA scFv F8. (A) Human and mouse EDA aminoacid sequence alignment. (B) Coomassie blue staining of the polyacrylamide gel with the purified scFv F8. (C, D) Recognition of human and mouse EDA proteins coated to ELISA plates by scFv F8 antibody fragment. (D) Titration experiment for EDA recognition using different human or mouse EDA concentrations coated to the ELISA plates. (E) Analysis of equilibrium dissociation constant (KD) of scFv F8 to soluble EDAs by using Bio-Layer Interferometry (BLI) technique.

FIG. 14. IFN-gamma production by EDA CAR-T cells after 48 hours of stimulation with mouse EDA (m) or human EDA (h) protein coated plates at 5 μg/ml.

FIG. 15. EDA CAR-T cell proliferation after 48 hours of stimulation with mouse EDA (m) or human EDA (h) protein coated plates at 5 μg/ml.

FIG. 16. IFN-gamma production of EDA CAR-T cells in response to stimulation with PM299L-EDA expressing tumor cells. EDA CAR-T cells were stimulated with PM299L-EDA tumor cells at two different tumor/CART cell ratios. IFN-gamma production was measured by ELISPOT. IFN-gamma production by EDA CAR-T cells alone was used as a negative control (No tumor).

FIG. 17. EDA CAR-T cells delay F9 teratocarcinoma tumor growth. 129Sv mice bearing F9 tumor cells (n=10 mice per group) were treated with a single dose of 1×10⁷ of F8 or C33 EDA CAR-T. Tumor growth at different time points is plotted.

FIG. 18. EDA expression in liver biopsies from primary and metastatic HCC (A) and in pancreatic biopsies from patients with PDAC (B), measured by immunohistochemistry using the anti EDA F8 ScFv.

FIG. 19. Antitumor effect of EDA CAR-T, EDB CAR-T or IIICS CAR-T cells in NSG mice xenotransplanted with the human hepatocarcinoma cell line PLC. Mean tumor area+/−SEM at different time points for each treatment is plotted.

### DETAILED DESCRIPTION OF THE INVENTION

### Definitions

“Administering” or “administration of” a medicament to a patient (and grammatical equivalents of this phrase) refers to direct administration, which may be administration to a patient by a medical professional or may be self-administration, and/or indirect administration, which may be the act of prescribing a drug. E.g., a physician who instructs a patient to self-administer a medicament or provides a patient with a prescription for a drug is administering the drug to the patient.

The term “affibody” refers to a protein that is derived from the Z domain of protein A and that been engineered to bind to a specific target (see Frejd & Kim, 2017. Exp Mol Med. 49 (3): e306).

The term “antibody” refers to a molecule comprising at least one immunoglobulin domain that binds to, or is immunologically reactive with, a particular target. The term includes whole antibodies and any antigen binding portion or single chains thereof and combinations thereof; for instance, the term “antibody” in particular includes bivalent antibodies and bivalent bispecific antibodies.

A typical type of antibody comprises at least two heavy chains (“HC”) and two light chains (“LC”) interconnected by disulfide bonds.

Each “heavy chain” comprises a “heavy chain variable domain” (abbreviated herein as “VH”) and a “heavy chain constant domain” (abbreviated herein as “CH”). The heavy chain constant domain typically comprises three constants domains, CH1, CH2, and CH3.

Each “light chain” comprises a “light chain variable domain” (abbreviated herein as “VL”) and a “light chain constant domain” (“CL”). The light chain constant domain (CL) can be of the kappa type or of the lambda type. The VH and VL domains can be further subdivided into regions of hypervariability, termed Complementarity Determining Regions (“CDR”), interspersed with regions that are more conserved, termed “framework regions” (“FW”).

Each VH and VL is composed of three CDRs and four FWs, arranged from amino-terminus to carboxy-terminus in the following order: FW1, CDR1, FW2, CDR2, FW3, CDR3, FW4. The present disclosure inter alia presents VH and VL sequences as well as the subsequences corresponding to CDR1, CDR2, and CDR3.

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme) and Lefranc et al. 2003 Dev Comp Imunol. 27:55-77 (“IMGT” numbering scheme).

Accordingly, a person skilled in the art would understand that the sequences of FW1, FW2, FW3 and FW4 are equally disclosed. For a particular VH, FW1 is the subsequence between the N-terminus of the VH and the N-terminus of H-CDR1, FW2 is the subsequence between the C-terminus of H-CDR1 and the N-terminus of H-CDR2, FW3 is the subsequence between the C-terminus of H-CDR2 and the N-terminus of H-CDR3, and FW4 is the subsequence between the C-terminus of H-CDR3 and the C-terminus of the VH. Similarly, for a particular VL, FW1 is the subsequence between the N-terminus of the VL and the N-terminus of L-CDR1, FW2 is the subsequence between the C-terminus of L-CDR1 and the N-terminus of L-CDR2. FW3 is the subsequence between the C-terminus of L-CDR2 and the N-terminus of L-CDR3, and FW4 is the subsequence between the C-terminus of L-CDR3 and the C-terminus of the VL.

The variable domains of the heavy and light chains contain a region that interacts with a binding target, and this region interacting with a binding target is also referred to as an “antigen-binding site” or “antigen binding site” herein. The constant domains of the antibodies can mediate the binding of the antibody to host tissues or factors, including various cells of the immune system (e.g., effector cells) and the first component (C1q) of the classical complement system. Exemplary antibodies of the present disclosure include typical antibodies, but also bivalent fragments and variations thereof such as a F(ab′)2.

As used herein, the term “antibody” encompasses intact polyclonal antibodies, intact monoclonal antibodies, bivalent antibody fragments (such as F(ab′)2), multispecific antibodies such as bispecific antibodies, chimeric antibodies, humanized antibodies, human antibodies, and any other modified immunoglobulin molecule comprising an antigen binding site.

An antibody can be of any the five major classes (isotypes) of immunoglobulins: IgA, IgD, IgE, IgG, and IgM, or subclasses thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as therapeutic agents or diagnostic agents to form immunoconjugates.

The term “anticalin” refers to a protein that is derived from the lipocalin and that been engineered to bind to a specific target (see Skerra, 2008. FEBS J. 275 (11): 2677-83).

The term “anti-cancer treatment” or “anti-cancer therapy” as used herein may include any treatment to stop or prevent cancer, including but not limited to surgery, radiotherapy, anti-cancer agents and any other existing therapies or to be developed.

The term “anti-cancer agent” as used herein refers to any therapeutic agents useful in treating cancer. Examples of anti-cancer therapeutic agents include, but are limited to, e.g., chemotherapeutic agents, growth inhibitory agents, cytotoxic agents, anti-hormonal agents, agents used in radiation therapy, anti-angiogenesis agents, apoptotic agents, anti-tubulin agents, and other agents to treat cancer, such as anti-HER-2 antibodies (e.g., Herceptin®), anti-CD20 antibodies, an epidermal growth factor receptor (EGFR) antagonist (e.g., a tyrosine kinase inhibitor), HER1/EGFR inhibitor (e.g., erlotinib (Tarceva<®>)), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “antigen-binding fragment” or “Fab” refers to an antibody fragment comprising one constant and one variable domain of each of the heavy and light chain. A Fab fragment may be obtained by digesting an intact monoclonal antibody with papain.

The term “cancer” refers to a group of diseases, which can be defined as any abnormal benign or malignant new growth of tissue that possesses no physiological function and arises from uncontrolled usually rapid cellular proliferation and has the potential to invade or spread to other parts of the body. Examples of cancer include but are not limited to, carcinoma, lymphoma, blastoma, sarcoma, and leukemia. More particular examples of such cancers include breast cancer, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, colon cancer, colorectal cancer, rectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The term “chimeric antigen receptor” or “CAR” refers to a synthetic receptor that targets T cells to a chosen antigen and reprograms T cell function, metabolism and persistence (Rivière & Sadelain, 2017). Similarly, the term “CART” refers to a T cell that comprises a CAR.

A “coding sequence” or a sequence which “encodes” a gene product as used herein, refers to a nucleic acid sequence which is transcribed (in the case of DNA) and translated (in the case of mRNA), in vitro or in vivo when placed under the control of appropriate regulatory sequences.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, three or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

A “complete response” or “complete remission” or “CR” indicates the disappearance of all target lesions as defined in the RECIST v1.1 guideline. This does not always mean the cancer has been cured.

The terms DNA “control sequences” and “control elements” as used herein, refer collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences/elements need always be present so long as the selected coding sequence is capable of being replicated, transcribed and translated in an appropriate host cell.

The term “costimulatory signaling domain” refers to a signaling moiety that provides to T cells a signal which, in addition to the primary signal provided by for instance the CD34 chain of the TCR/CD3 complex, mediates a T cell response, including, but not limited to, activation, proliferation, differentiation, cytokine secretion, and the like. A co-stimulatory domain can include all or a portion of, but is not limited to, CD27, CD28, 4-1BB (CD137), OX40 (CD134), CD30, CD40, 1COS, lymphocyte function-associated antigen-1 (LFA-1), CD2, CD7, LIGHT, NKG2C, B7-H3, and a ligand that specifically binds with CD83. In some embodiments, the co-stimulatory signaling domain is an intracellular signaling domain that interacts with other intracellular mediators to mediate a cell response including activation, proliferation, differentiation and cytokine secretion, and the like.

The term “designed ankyrin repeat proteins” or “DARPin” refers to a protein that is derived from an ankyrin repeat that has been engineered to bind to a specific target (see Pluckthun, 2015. Annu Rev Pharmacol Toxicol. 55:489-511).

“Disease free survival” (DFS) refers to the length of time during and after treatment that the patient remains free of disease.

The term “dosage form” refers to a pharmaceutical composition devised to enable administration of a drug medication in the prescribed dosage amounts. Depending on the method/route of administration different dosage forms will be used. Oral dosage forms comprise liquids (i.e., solutions, suspensions, and emulsions), semi-solids (i.e., pastes), and solids (i.e., tablets, capsules, powders, granules, premixes, and medicated blocks), these may be immediate release or modified release forms. Parenteral dosage forms and delivery systems include injectables (i.e., solutions, suspensions, emulsions, and dry powders for reconstitution), intramammary infusions, intravaginal delivery systems, and implants. Topical dosage forms include solids (i.e., dusting powders), semi-solids (i.e., creams, ointments, and pastes), and liquids (i.e., solutions, suspension concentrates, suspoemulsions, and emulsifiable concentrates). It further includes transdermal delivery systems.

The term “EDA” refers to an isoform of fibronectin (FN) arising from alternative splicing which incorporates the extra domain A (also referred as Extra-domain-A fibronectin or EDA-FN) and also specifically to this extra domain. FN molecules have multiple isoforms generated from a single gene by alternative splicing of combinations of 3 exons: extra domain-A (EDA), extra domain-B (EDB), and III-CS. Both EDA and EDB exons are type III repeating units. Cellular FNs (cFNs), many of which are insoluble and incorporated into the pericellular matrix, contain the EDA and EDB segments in various combinations (Saito et al., 1999). This isoform is expressed during embryonic development, restricted in normal adult tissues and re-expressed in adults during cancer progression (Wang J P et al., 2017). EDA has been reported to be strongly expressed in many tumor types as compared to normal tissues (reviewed in Kumra H et al., 2016), and this increased expression has been correlated with cancer progression, dissemination and poor prognosis. Exemplary sequence and data related to human EDA domain has been deposited in the RCSB Protein Databank under ID number 1J8K. The NMR structure of the fibronectin EDA domain is available at https://www.rcsb.org/structure/1j8k.

“EDA-positive” cancer, including a “EDA-positive” cancerous disease, is one comprising EDA as part of the extracellular matrix (ECM) in the tumor microenvironment (TME). The term “EDA-positive” also refers to a cancer that produces sufficient levels of EDA in the TME, such that a CAR-comprising cell of the present invention has a therapeutic effect, mediated by the binding of the CAR to EDA. In some embodiments, the EDA-positive cancer is colorectal cancer, liver cancer, pancreatic cancer, breast cancer, ovary cancer, prostate cancer, testis cancer, bladder cancer, glioma, melanoma, lymphoma, head and neck cancer, or cholangiocarcinoma.

The term “EDA-targeting moiety” refers to a substance that can bind EDA. Within the context of a CAR, an EDA-targeting moiety targets the CAR expressing cells, such as T cells, to an EDA-positive tumor. Within the context of a CAR, it is to be understood that the EDA-targeting moiety is genetically encodable.

As used herein, the term “effective amount” of an agent, e.g., a therapeutic agent such as a CART, is that amount sufficient to effect beneficial or desired results, for example, clinical results, and, as such, an “effective amount” depends upon the context in which it is being applied. For example, in the context of administering a therapeutic agent that treats cancer, an effective amount can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof. The term “effective amount” can be used interchangeably with “effective dose,” “therapeutically effective amount,” or “therapeutically effective dose”.

The term “fynomer” refers to a protein that is derived from the SH3 domain of human Fyn kinase that has been engineered to bind to a specific target (see Bertschinger et al., 2007. Protein Eng Des Sel. 20 (2): 57-68).

“Infusion” or “infusing” refers to the introduction of a therapeutic agent-containing solution into the body through a vein for therapeutic purposes. Generally, this is achieved via an intravenous bag.

“Intracellular signaling domain” as used herein refers to all or a portion of one or more domains of a molecule (here the chimeric receptor molecule) that provides for activation of a lymphocyte. Intracellular domains of such molecules mediate a signal by interacting with cellular mediators to result in proliferation, differentiation, activation and other effector functions. Examples of intracellular signaling domains for use in a CAR of the invention as described herein include the intracellular sequences of the CD3ζ chain, and/or co-receptors that act in concert to initiate signal transduction following CAR engagement, as well as any derivative or variant of these sequences and any synthetic sequence that has the same functional capability. T cell activation can be said to be mediated by two distinct classes of cytoplasmic signaling sequence: those that initiate antigen-dependent primary activation and provide a T cell receptor like signal (primary cytoplasmic signaling sequences) and those that act in an antigen-independent manner to provide a secondary or co-stimulatory signal (secondary cytoplasmic signaling sequences). Primary cytoplasmic signaling sequences that act in a stimulatory manner may contain signaling motifs which are known as receptor tyrosine-based activation motifs or ITAMs. Examples of ITAM containing primary cytoplasmic signaling sequences include those derived from CD3ζ, FcRγ, CD3γ, CD3δ, CD3ε, CD5, CD22, CD79a, CD79b, and CD66d.

The term “metastasis” as used herein refers to distant metastasis affecting organs other than the primary tumor site. Metastasis may be defined as the process by which cancer spreads or transfers from the primary site to other regions of the body with the development of a similar cancerous lesion at the new location (see for instance: Chambers A F et al., Nat Rev Cancer 2002; 2:563-72). For instance, in colorectal cancer, metastasis in another organ (e.g., the liver) typically shows an enteroid adenocarcinoma pattern. A “metastatic” or “metastasizing” cell is typically one that loses adhesive contacts with neighboring cells and migrates via the bloodstream or lymph from the primary site of disease to invade neighboring body structures.

The term “monobody” refers to a protein that is derived from a fibronectin type III domain that has been engineered to bind to a specific target (see Koide et al., 2013. J Mol Biol. 415 (2): 393-405).

The term “nanobody” refers to a protein comprising the soluble single antigen-binding V-domain of a heavy chain antibody, preferably a camelid heavy chain antibody (see Bannas et al., 2017. Front Immunol. 8:1603).

“Operably linked” as used herein refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of diagnosis or treatment.

A “partial response” or “PR” refers to at least a 30% decrease in the sum of diameters of target lesions, taking as reference the baseline sum diameter, in response to treatment, as defined in the RECIST v1.1 guideline (European Journal of Cancers 45 (2009) 228-247) or in the iRECIST guideline for response criteria for use in trials testing immunotherapeutics (Seymour et al, Lancet Oncol. 2017 March; 18 (3): e143-e152).

The term “peptide aptamer” refers to a short, 5-20 amino acid residue sequence that can bind to a specific target. Peptide aptamers are typically inserted within a loop region of a stable protein scaffold (see Reverdatto et al., 2015. Curr Top Med Chem. 15 (12): 1082-101).

As used herein, “pharmaceutically acceptable excipient”, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington's Pharmaceutical Sciences 16th edition, Osol, A. Ed. (1980) may also be included in a pharmaceutical composition described herein, provided that they do not adversely affect the desired characteristics of the pharmaceutical composition.

“Progressive disease” or “disease that has progressed” refers to the appearance of one more new lesions or tumors and/or the unequivocal progression of existing non-target lesions as defined in the RECIST v1.1 guideline (European Journal of Cancers 45 (2009) 228-247) or in the iRECIST guideline for response criteria for use in trials testing immunotherapeutics (Seymour et al, Lancet Oncol. 2017 March; 18 (3): e143-e152). Progressive disease or disease that has progressed can also refer to a tumor growth of more than 20 percent since treatment began, either due to an increase in mass or in spread of the tumor.

“Progression free survival” (PFS) refers to the time from enrollment to disease progression or death. PFS is generally measured using the Kaplan-Meier method and Response Evaluation Criteria in Solid Tumors (RECIST) 1.1 standards (European Journal of Cancers 45 (2009) 228-247) or in the iRECIST guideline for response criteria for use in trials testing immunotherapeutics (Seymour et al, Lancet Oncol. 2017 March; 18 (3): e143-e152). Generally, progression free survival refers to the situation wherein a patient remains alive, without the cancer getting worse.

The term “promoter” as used herein refers to a region of DNA that initiates transcription of a particular coding sequence. Promoters are located near the transcription start sites of genes, on the same strand and upstream on the DNA (towards the 5′ region of the sense strand).

Promoters can be about 100-1000 base pairs long. An “eukaryotic promoter” includes cis-acting elements such as binding sites for activating protein-1 (AP-1), nuclear factor κB (NF-κB), CArG binding factor A (CBF-A), nuclear factor Y (NF-Y) and others, in addition to the TATA box sequence.

The term “repebody” refers to a protein that is derived from a leucine-rich repeat module and that been engineered to bind to a specific target (see Lee et al., 2012. PNAS. 109 (9): 3299-3304).

The term “respond favorably” generally refers to causing a beneficial state in a subject. With respect to cancer treatment, the term refers to providing a therapeutic effect on the subject. Positive therapeutic effects in cancer can be measured in a number of ways (See, Weber, 2009. J Nucl Med. 50 Suppl 1: 1S-10S). For example, tumor growth inhibition, molecular marker expression, serum marker expression, and molecular imaging techniques can all be used to assess therapeutic efficacy of an anti-cancer therapeutic. With respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. A favorable response can be assessed, for example, by increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof.

The term “sequence identity” refers to a percentage value obtained when two sequences are compared using a pairwise sequence alignment tool. In the present case, the sequence identity is obtained using the global alignment tool “EMBOSS Needle” using the default settings (Rice et al., 2000. Trends Genet. 16 (6): 276-7; Li et al., 2015. Nucleic Acids Res. 43(W1): W580-4). The global alignment tool is available at: https://www.ebi.ac.uk/Tools/psa/.

The term “variant” refers to an amino acid molecule having a percentage of sequence identity with a (poly) peptide. Preferably, variants described herein are characterized by being able to perform the biological function of the naturally occurring or canonical sequence of the (poly) peptide.

The term “signal peptide” as used herein refers to an amino acid sequence which permits the secretion of a recombinant polypeptide from the cell and cleavage of the signal peptide. The signal peptide has typically from 15 to 30 amino acid residues but may have up to 50 amino acids, and is usually located at the amino-terminus. A person skilled in the art will know how to select the most appropriate signal peptide. The best choice for a signal peptide sequence may be the proteins native signal peptide unless truncations from the amino terminus are to be explored. In either case, testing a small panel of commonly utilized signal sequences may be desirable. A handful of efficient and well-described signal sequences include interleukin-2, CD5, the Immunoglobulin Kappa light chain, trypsinogen, serum albumin, and prolactin, although there are many others that have proved beneficial as well (Stern et al. Trends Cell Mol Biol 2:1-17; Kober et al., Biotechnol Bioeng 110:1164-1173).

The term “single-chain antigen-binding fragment” or “scFab” refers to a fusion protein comprising one variable and one constant domain of the light chain of an antibody attached to one variable and one constant domain of the heavy chain of an antibody, wherein the heavy and light chains are linked together through a short peptide.

The term “single-chain variable fragment” or “scFv” refers to a fusion protein comprising the variable domains of the heavy chain and light chain of an antibody linked to one another with a peptide linker, preferably a 14 to 20 amino acid linker. Suitable linkers are known in the art and available to the skilled person. The scFv has two possible structures, with the VH at the N-termini or with the VL at the N-termini. The term also includes a disulfide stabilized Fv (dsFv).

Methods of stabilizing scFvs with disulfide bonds are disclosed in Reiter et al., 1996. Nat Biotechnol. 14 (10): 1239-45.

“Stable disease” refers to disease without progression or relapse as defined in the RECIST v1.1 guideline (European Journal of Cancers 45 (2009) 228-247) or in the iRECIST guideline for response criteria for use in trials testing immunotherapeutics (Seymour et al, Lancet Oncol. 2017 March; 18(3):e143-e152). In stable disease there is neither sufficient tumor shrinkage to qualify for partial response, nor sufficient tumor increase to qualify as progressive disease.

The term “stroma” as used herein refers to the extracellular matrix (ECM), which is composed of proteoglycans, hyaluronic acid, and fibrous proteins such as collagen, fibronectin, and laminin; as well as to growth factors, chemokines, cytokines, antibodies, and metabolites; and mesenchymal supporting cells (e.g., fibroblasts and adipocytes), cells of the vascular system, and cells of the immune system.

The term “subject” as used herein refers to a mammalian subject. Preferably, it is selected from a human, companion animal, non-domestic livestock or zoo animal. For example, the subject may be selected from a human, dog, cat, cow, pig, sheep, horse, bear, and so on. In a preferred embodiment, said mammalian subject is a human subject. In preferred embodiments, the term “subject” is used to designate a human being and is not meant to be limiting in any way. The terms “individual”, “patient” or “subject” may be used interchangeably in the present application. The “individual”, “patient” or “subject” can be of any age, sex and physical condition. The term “patient in need thereof” usually refers to a patient who suffers from a EDA-positive cancer.

“Time to Tumor Progression” (TTP) is defined as the time from enrollment to disease progression. TTP is generally measured using the RECIST v1.1 criteria (European Journal of Cancers 45 (2009) 228-247) or the iRECIST criteria for use in trials testing immunotherapeutics (Seymour et al, Lancet Oncol. 2017 March; 18(3):e143-e152).

The terms “treatment” and “therapy”, as used in the present application, refer to a set of hygienic, pharmacological, surgical and/or physical means used with the intent to cure and/or alleviate a disease and/or symptoms with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.

### DETAILED DESCRIPTION

### Chimeric Receptor Nucleic Acid and Polypeptides of the Invention

A first aspect of the invention relates to a chimeric antigen receptor (CAR)—also referred herein a chimeric receptor—nucleic acid comprising a polynucleotide coding for a ligand binding domain comprising an extra-domain A of fibronectin (EDA) targeting-moiety, optionally, a polynucleotide coding for a hinge or spacer region, a polynucleotide coding for a transmembrane domain; and a polynucleotide coding for an intracellular signaling domain.

### Ligand Binding Domain

In some embodiments, the EDA-targeting moiety can be an antibody or antibody fragment (e.g. scFv, Fab or scFab), anticalin, repebody, monobody, affibody, fynomer, DARPin, nanobody, or peptide aptamer that specifically binds to EDA.

Binding molecules that bind specifically to EDA of fibronectin are known in the art. Exemplary human antibodies against EDA are the F8, B7 and D5 antibodies described by Villa et al. 2008, see also Rybak J N et al. 2007 and US20180079793 A1; or the IST-9 antibody described by Carnemolla et al., 1987. Other antibodies specifically binding to EDA have been described in WO2015/088348 A1, such as antibodies 17G8.72, 27A12.70, 29E7.35, 42H11.51 and 33E3.10. In some embodiments, said binding domain binds specifically to mouse EDA (SEQ ID NO: 29) and/or human EDA (SEQ ID NO: 30). In preferred embodiments, said binding domain is cross-reactive to mouse and human EDA protein, such as the F8 antibody or the 33E3.10 antibody and antigen binding fragments thereof.

It is also known in the art that antibodies that specifically bind an antigen, such as a tumor cell surface molecule, can be prepared using methods of obtaining monoclonal antibodies, methods of phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. In some embodiments, antibodies specifically bind to a tumor cell surface molecule and do not cross react with nonspecific components such as bovine serum albumin or other unrelated antigens. Once identified, the amino acid sequence or polynucleotide sequence coding for the antibody can be isolated and/or determined.

Antibodies or antigen binding fragments include all or a portion of polyclonal antibodies, a monoclonal antibody, a human antibody, a humanized antibody, a synthetic antibody, a chimeric antibody, a bispecific antibody, a minibody, and a linear antibody. Antibody fragments comprise a portion of an intact antibody, preferably the antigen binding or variable region of the intact antibody and can readily be prepared. Examples of antibody fragments include Fab, Fab′, F(ab′)₂, and Fv fragments; diabodies; linear antibodies; single-chain antibody molecules; and multispecific antibodies formed from antibody fragments.

A number of different antibodies that bind to EDA can be isolated and characterized. In some embodiments, the antibodies are characterized based on epitope specificity of the targeted molecule. In addition, in some cases, antibodies that bind to the same epitope can be selected based on the affinity of the antibody for that epitope. In some embodiments, an antibody has an affinity of at least 1 mM, and preferably <50 nM. In some embodiments, an antibody is selected that has a higher affinity for the epitope as compared to other antibodies. For example, an antibody is selected that has at least a 2 fold, at least a 5 fold, at least a 10 fold, at least a 20 fold, at least a 30 fold, at least a 40 fold, or at least a 50 fold greater affinity than a reference antibody that binds to the same epitope.

Methods of generating and selecting non-immunoglobulin scaffolds that bind to a particular target are also known in the art (see, for example, Škrlec, et al., 2015. Trends Biotechnol. 33(7):408-18).

In some embodiments, the EDA-targeting moiety is an antibody, scFv, Fab, or scFab comprising a VH domain and VL domain, wherein said VH domain comprises a HCDR1 comprising or consisting of [GFTFSVM] (SEQ ID NO: 1), HCDR2 comprising or consisting of [SGSGGS] and HCDR3 comprising or consisting of [STHLYLFDY] (SEQ ID NO: 3); and said VL domain comprises a LCDR1 comprising or consisting of [RASQSVSNAFLA] (SEQ ID NO: 4), LCDR2 comprising or consisting of [GASSRAT] (SEQ ID NO: 5), and LCDR3 comprising or consisting of [QQMRGRPPT] (SEQ ID NO: 6).

In some embodiments, the EDA-targeting moiety is an antibody, scFv, Fab, or scFab, preferably a scFv, comprising a VH domain and VL domain, wherein the VH domain comprises or consists of SEQ ID NO: 7 and the VL domain comprises or consists of SEQ ID NO: 8.

VH domain (SEQ ID NO: 7):
[EVQLLESGGGLVQPGGSLRLSCAASGFTFSVMKMSWVRQAPGKGLEWV
SAISGSGGSTYYADSVKGRFTISRDNSKNTLYLQMNSLRAEDTAVYYCA
KSTHLYLFDYWGQGTLVTVSS]
VL domain (SEQ ID NO: 8):
[EIVLTQSPGTLSLSPGERATLSCRASQSVSNAFLAWYQQKPGQAPRLL
IYGASSRATGIPDRFSGSGSGTDFTLTISRLEPEDFAVYYCQQMRGRPP
TFGQGTKVEIK].

In a preferred embodiment of any of the above, said EDA-targeting moiety is a human or humanized scFv. In some embodiments, the VL domain is at the N-terminal end and the VH domain at the C-terminal end. In other embodiments, the VH domain is at the N-terminal end and the VL domain at the C-terminal end.

As defined above, the term “single-chain variable fragment” or “scFv” refers to a fusion protein comprising the variable domains of the heavy chain and light chain of an antibody linked to one another with a peptide linker. The term “peptide linker”, “linker” or “spacer” as used herein refers to a spacer acting as a hinge region between polypeptide domains, allowing them to move independently from one another while maintaining the three-dimensional form of the individual domains. In this sense, a preferred spacer would be a hinge region characterized by a structural ductility or flexibility allowing this movement.

The length of the spacer can vary; typically, the number of amino acids in the spacer is 100 or less amino acids, preferably 50 or less amino acids, more preferably 40 or less amino acids, still more preferably, 30 or less amino acids, or even more preferably 20 or less amino acids. Preferred ranges are from 5 to 50, preferably from 10 to 30, more preferably from 15 to 25 amino acids. In a preferred embodiment the length of the spacer is of about 15 or about 20 amino acids. In another preferred embodiment, the length of the spacer is of around 20 amino acids.

In preferred embodiments, said spacer is a peptide having structural flexibility (i.e., a flexible linking peptide or “flexible linker”) and comprises 2 or more amino acids selected from the group consisting of glycine, serine, alanine and threonine. Preferably, wherein at least 65%, preferably 70%, 75%, 80%, 85%, 90%, 95%, 96%, 97%, 98%, 99% or 100% of the amino acids in said flexible peptide linker are selected from the group consisting of glycine, serine, alanine and threonine.

The spacer peptide may preferably contain repeats of amino acid residues, particularly Gly and Ser, or any other suitable repeats of amino acid residues. Regardless the presence or absence of repeats, it is also preferred that at least 65%, preferably 70%, 75%, 80%, 85%, 90%, 95%, 96%, 97%, 98%, 99% or 100% of the amino acids in the peptide linker are selected from the group consisting of Gly and Ser.

Non-limiting examples of linkers are disclosed in Shen et al., Anal. Chern. 80 (6): 1910-1917 (2008) and WO 2014/087010. Various linker sequences are known in the art, including, without limitation, glycine serine (GS) linkers such as (GS)n, (GSGGS)n, (GGGS)n, and (GGGGS)n, where n represents an integer of at least 1, preferably between 1 and 5. In preferred embodiments, said linker comprises or consists of (GGGGS)n, where n represents an integer of at least 1, preferably between 1 and 5; preferably comprises or consists of SEQ ID NO: 23.

In some embodiments, the EDA-targeting moiety is a scFv comprising or consisting of SEQ ID NO: 9.

anti-EDA F8 scFv (SEQ ID NO: 9):
[EVQLLESGGGLVQPGGSLRLSCAASGFTFSVMKMSWVRQAPGKGLEWV
SAISGSGGSTYYADSVKGRFTISRDNSKNTLYLQMNSLRAEDTAVYYCA
KSTHLYLFDYWGQGTLVTVSSGGGGSGGGGSGGGGSGGGGSEIVLTQSP
GTLSLSPGERATLSCRASQSVSNAFLAWYQQKPGQAPRLLIYGASSRAT
GIPDRFSGSGSGTDFTLTISRLEPEDFAVYYCQQMRGRPPTFGQGTKVE
IK]
CDRs underlined and linker (SEQ ID NO: 23:
GGGGGGGGSGGGGSGGGGS) in bold.

In some embodiments, the EDA-targeting moiety is a scFv comprising a VH domain and VL domain, wherein the VH and VL domain comprise the corresponding CDRs of the F8 scFv as determined by the Kabat numbering scheme. For instance, antibody amino acid residue positions in F8 ScFv are assigned using ANARCI (SAbPred: ANARCI (ox.ac.uk)), and alignments are done with the Kabat antibody numbering schemes. CDR annotations are assigned using SCALOP (SAbPred: SCALOP (ox.ac.uk)). In other embodiments, the EDA-targeting moiety is a scFv comprising variable regions that have at least 90% amino acid sequence identity to that of the F8 scFv and that have at least the same affinity for EDA.

In other embodiments, the EDA-targeting moiety is an antibody, scFv, Fab, or scFab, preferably a scFv, comprising the six CDRs or the VH domain and VL domain of the 27A12.70 antibody described in WO2015/088348 A1. In a particular embodiment, the EDA-targeting moiety is a scFv comprising or consisting of SEQ ID NO: 26 (scFv clone 27).

In still other embodiments, the EDA-targeting moiety is an antibody, scFv, Fab, or scFab, preferably a scFv, comprising the six CDRs or the VH domain and VL domain of the 33E3.10 antibody described in WO2015/088348 A1. In a particular embodiment, the EDA-targeting moiety is a scFv comprising or consisting of SEQ ID NO: 28 (scFv clone 33).

### Hinge or Spacer Region

The (EDA) targeting-moiety can be linked at its C-terminal end to a hinge or spacer region. The hinge or spacer region is defined as the extracellular structural region that extends the binding units from the transmembrane domain. The hinge provides flexibility which helps to overcome steric hindrance and contributes to the length in order to allow the antigen-binding domain to access the targeted epitope. The length of the hinge region can vary. Typically, smaller target antigens will be associated with longer hinge regions. In some embodiments, the hinge region has from 10 to 100 amino acids, preferably from 20 to 80, from 30 to 70, from 40 to 60 or from 45 to 55 amino acids in length. In a preferred embodiment, the hinge region has 45 amino acids. The hinge region may for instance be derived from the amino acid sequence of the transmembrane domain as defined herein below, commonly employed hinge regions are derived from amino acid sequences from CD8, CD28 or from the Fc fragment of IgG1 or IgG4.

In some embodiments, the hinge region comprises or consists of the hinge region of CD8, preferably human CD8 (SEQ ID NO: 10) or a sequence that has at least 70%, 75%, 80%, 85%, 90%, 95%, 96%, 97%, 98%, 99% or 100% sequence identity thereto. In some embodiments, the hinge region comprises or consists of the hinge region of CD8, preferably human CD8 (SEQ ID NO: 10) or a sequence that has at least 95% sequence identity thereto. In some embodiments, the hinge region comprises or consists of SEQ ID NO: 10 or a sequence that has at least 98% sequence identity thereto. In some embodiments, the hinge region comprises or consists of SEQ ID NO: 10 or a sequence that has at least 99% sequence identity thereto.

Hinge region of human CD8 (SEQ ID NO: 10):
TTTPAPRPPTPAPTIASQPLSLRPEACRPAAGGAVHTRGLDFACD

### Transmembrane Domain

The transmembrane domain is involved in the anchoring of the chimeric receptor in the cell membrane. The transmembrane domain may be derived either from a natural or a synthetic source. When the source is natural, the domain may be derived from any membrane-bound or transmembrane protein. Transmembrane regions may comprise or consist of at least the transmembrane region(s) of the α-, β- or ζ-chain of CD28, CD3, CD45, CD4, CD8, CD9, CD16, CD22, CD33, CD37, CD64, CD80, CD86, CD134, CD137, or CD154.

A transmembrane domain may be synthetic or a variant of a naturally occurring transmembrane domain. In some embodiments, synthetic or variant transmembrane domains comprise predominantly hydrophobic residues such as leucine and valine. In some embodiments, a transmembrane domain has at least 70%, 75%, 80%, 85%, 90%, 95%, 96%, 97%, 98%, 99% or 100% identity to the naturally occurring transmembrane domain, such as CD28, CD3, CD45, CD4, CD8, CD9, CD16, CD22, CD33, CD37, CD64, CD80, CD86, CD134, CD137 and CD154, preferably CD8.

In some embodiments, the transmembrane domain comprises or consists of the transmembrane domain of CD28, CD3, CD45, CD4, CD8, CD9, CD16, CD22, CD33, CD37, CD64, CD80, CD86, CD134, CD137, CD154, preferably CD8, or a variant of any thereof, wherein the variant thereof has at least 95% sequence identity.

In some embodiments, the transmembrane domain comprises or consists of the transmembrane domain of CD28, CD3, CD45, CD4, CD8, CD9, CD16, CD22, CD33, CD37, CD64, CD80, CD86, CD134, CD137, CD154, preferably CD8, or a variant of any thereof, wherein the variant thereof has at least 98% sequence identity.

In some embodiments, the transmembrane domain comprises or consists of the transmembrane domain of CD28, CD3, CD45, CD4, CD8, CD9, CD16, CD22, CD33, CD37, CD64, CD80, CD86, CD134, CD137, or CD154. Preferably, the CD28, CD3, CD45, CD4, CD8, CD9, CD16, CD22, CD33, CD37, CD64, CD80, CD86, CD134, CD137, or CD154 protein as referred herein is the corresponding human or murine protein. In preferred embodiments, the transmembrane domain comprises or consists of the transmembrane domain of CD8, preferably human CD8 (SEQ ID NO: 11).

Transmembrane domain of human CD8 (SEQ ID NO: 11)
[IYIWAPLAGTCGVLLLSLVITLYC]

### Intracellular Signaling Domain

The intracellular signaling domain provides for the activation of at least one function of the cell expressing the CAR upon binding to the ligand expressed on tumor cells. In some embodiments, the intracellular signaling domain contains one or more intracellular signaling domains. In some embodiments, the intracellular signaling domain is a portion of and/or a variant of an intracellular signaling domain that provides for activation of at least one function of the CAR-comprising cell. In some embodiments, the intracellular signaling domain has at least 70%, 75%, 80%, 85%, 90%, 95%, 96%, 97%, 98%, 99% or 100% identity to the intracellular domain of CD3ζ, FcRγ, CD3γ, CD3δ, CD3ε, CD5, CD22, CD79a, CD79b, CD66b, preferably of CD3ζ.

In some embodiments, the intracellular signaling domain comprises or consists of the intracellular domain of CD3ζ, FcRγ, CD3γ, CD3δ, CD3ε, CD5, CD22, CD79a, CD79b, CD66b, or a variant thereof, wherein the variant thereof has at least 95% sequence identity.

In some embodiments, the intracellular signaling domain comprises or consists of the intracellular domain of CD3ζ, FcRγ, CD3γ, CD3δ, CD3ε, CD5, CD22, CD79a, CD79b, CD66b, or a variant thereof, wherein the variant thereof has at least 98% sequence identity.

In some embodiments, the intracellular signaling domain comprises the intracellular domain of CD3ζ, FcRγ, CD3γ, CD3δ, CD3ε, CD5, CD22, CD79a, CD79b or CD66b. Preferably, the CD3ζ, FcRγ, CD3γ, CD3δ, CD3ε, CD5, CD22, CD79a, CD79b, CD66b protein as referred herein is the corresponding human or murine protein. In preferred embodiments, the intracellular signaling domain comprises or consists of the intracellular domain of CD3ζ, preferably human CD3ζ (SEQ ID NO: 17) or a variant thereof having at least 70% identity, for instance a C-terminal truncated variant such as SEQ ID NO: 12.

Intracellular signaling domain of human CD3ζ
(SEQ ID NO: 12)
[RVKFSRSADAPAYQQGQNQLYNELNLGRREEYDVLDKRRGRDPEMGG
KPRRKNPQEGLYNELQKDKMAEAYSEIGMKGERRRGKGHDGLYQGLST
AT]
Intracellular signaling domain of human CD3ζ-
complete sequence
(SEQ ID NO: 17)
[RVKFSRSADAPAYQQGQNQLYNELNLGRREEYDVLDKRRGRDPEMGG
KPRRKNPQEGLYNELQKDKMAEAYSEIGMKGERRRGKGHDGLYQGLST
ATKDTYDALHMQALPPR]

### Costimulatory Signaling Domain

In some embodiments, the CAR may further comprise a costimulatory signaling domain. In some embodiments, the costimulatory signaling domain comprises or consists of the intracellular domain of CD27, CD28, CD137, CD134, CD30, CD40, lymphocyte function-associated antigen-1 (LFA-1), CD2, CD7, LIGHT, NKG2C, CD276, preferably CD137, or a variant of any thereof, wherein the variant thereof has at least 70%, 75%, 80%, 85%, 90%, 95%, 96%, 97%, 98%, 99% or 100% identity thereto.

In some embodiments, the costimulatory signaling domain comprises or consists of the intracellular domain of CD27, CD28, CD137, CD134, CD30, CD40, lymphocyte function-associated antigen-1 (LFA-1), CD2, CD7, LIGHT, NKG2C, CD276, preferably CD137, or a variant of any thereof, wherein the variant thereof has at least 95% sequence identity.

In some embodiments, the costimulatory signaling domain comprises or consists of the intracellular domain of CD27, CD28, CD137, CD134, CD30, CD40, lymphocyte function-associated antigen-1 (LFA-1), CD2, CD7, LIGHT, NKG2C, CD276, preferably CD137, or a variant of any thereof, wherein the variant thereof has at least 98% sequence identity.

In some embodiments, the costimulatory signaling domain comprises or consists of the intracellular domain of CD27, CD28, CD137, CD134, CD30, CD40, lymphocyte function-associated antigen-1 (LFA-1), CD2, CD7, LIGHT, NKG2C, or CD276. Preferably, the CD27, CD28, CD137, CD134, CD30, CD40, lymphocyte function-associated antigen-1 (LFA-1), CD2, CD7, LIGHT, NKG2C, or CD276 protein as referred herein is the corresponding human or murine protein. In preferred embodiments, the costimulatory signaling domain comprises or consist of the intracellular domain of CD137, preferably human CD137 (SEQ ID NO: 13).

Costimulatory signaling domain of CD137
(SEQ ID NO: 13)
[KRGRKKLLYIFKQPFMRPVQTTQEEDGCSCRFPEEEEGGCEL]

### Full Sequence CARs According to the Present Invention

In some embodiments, the CAR comprises:
(i) an EDA-targeting moiety as described herein;
(ii) optionally, a hinge or spacer region comprising or consisting of SEQ ID NO: 10 or a sequence that has at least 95% sequence identity thereto;
(iii) a transmembrane domain comprising or consisting of SEQ ID NO: 11 or a sequence that has at least 95% sequence identity thereto;
(iv) an intracellular signaling domain comprising or consisting of SEQ ID NO: 12, SEQ ID NO: 17 or a sequence that has at least 95% sequence identity to any thereto; and
(v) a costimulatory signaling domain comprising or consisting of SEQ ID NO: 13 or a sequence that has at least 95% sequence identity thereto.

In some embodiments, the CAR comprises:
(i) an EDA-targeting moiety as described herein;
(ii) optionally, a hinge or spacer region comprising or consisting of SEQ ID NO: 10 or a sequence that has at least 98% sequence identity thereto;
(iii) a transmembrane domain comprising or consisting of SEQ ID NO: 11 or a sequence that has at least a 98% sequence identity thereto;
(iv) an intracellular signaling domain comprising or consisting of SEQ ID NO: 12, SEQ ID NO: 17 or a sequence that has at least a 98% sequence identity to any thereto; and
(v) a costimulatory signaling domain comprising or consisting of SEQ ID NO: 13 or a sequence that has at least a 98% sequence identity thereto.

In some embodiments, the CAR comprises:
(i) an EDA-targeting moiety as described herein;
(ii) optionally, a hinge or spacer region comprising or consisting of SEQ ID NO: 10 or a sequence that has at least 99% sequence identity thereto;
(iii) a transmembrane domain comprising or consisting of SEQ ID NO: 11 or a sequence that has at least 99% sequence identity thereto;
(iv) an intracellular signaling domain comprising or consisting of SEQ ID NO: 12, SEQ ID NO: 17 or a sequence that has at least 99% sequence identity to any thereto; and
(v) a costimulatory signaling domain comprising or consisting of SEQ ID NO: 13 or a sequence that has at least 99% sequence identity to thereto.

In some embodiments, the CAR comprises:
(i) an EDA-targeting moiety as described herein;
(ii) optionally, a hinge or spacer region comprising SEQ ID NO: 10;
(iii) a transmembrane domain comprising of SEQ ID NO: 11;
(iv) an intracellular signaling domain comprising of SEQ ID NO: 12 or SEQ ID NO: 17; and
(v) a costimulatory signaling domain comprising SEQ ID NO: 13.

In some embodiments, the CAR comprises:
(i) an EDA-targeting moiety as described herein;
(ii) optionally, a hinge or spacer region consisting of SEQ ID NO: 10;
(iii) a transmembrane domain consisting of SEQ ID NO: 11;
(iv) an intracellular signaling domain consisting of SEQ ID NO: 12 or SEQ ID NO: 17; and
(v) a costimulatory signaling domain consisting of SEQ ID NO: 13.

In some embodiments, the CAR comprises:
(i) a scFv comprising a VH domain and VL domain, wherein the VH domain comprises a HCDR1 consisting of SEQ ID NO: 1, a HCDR2 consisting of SEQ ID NO: 2 and a HCDR3 consisting of SEQ ID NO: 3; and the VL domain comprises a LCDR1 consisting of SEQ ID NO: 4, a LCDR2 consisting of SEQ ID NO: 5 and a LCDR3 consisting of SEQ ID NO: 6;
(ii) optionally, a hinge or spacer region comprising or consisting of SEQ ID NO: 10 or a sequence that has at least 95% sequence identity thereto;
(iii) a transmembrane domain comprising or consisting of SEQ ID NO: 11 or a sequence that has at least 95% sequence identity thereto;
(iv) an intracellular signaling domain comprising or consisting of SEQ ID NO: 12, SEQ ID NO: 17 or a sequence that has at least 95% sequence identity to any thereto; and
(v) a costimulatory signaling domain comprising or consisting of SEQ ID NO: 13 or a sequence that has at least 95% sequence identity thereto.

In some embodiments, the CAR comprises:
(i) a scFv comprising a VH domain and VL domain, wherein the VH domain comprises a HCDR1 consisting of SEQ ID NO: 1, a HCDR2 consisting of SEQ ID NO: 2 and a HCDR3 consisting of SEQ ID NO: 3; and the VL domain comprises a LCDR1 consisting of SEQ ID NO: 4, a LCDR2 consisting of SEQ ID NO: 5 and a LCDR3 consisting of SEQ ID NO: 6;
(ii) optionally, a hinge or spacer region comprising or consisting of SEQ ID NO: 10 or a sequence that has at least 98% sequence identity thereto;
(iii) a transmembrane domain comprising or consisting of SEQ ID NO: 11 or a sequence that has at least a 98% sequence identity thereto;
(iv) an intracellular signaling domain comprising or consisting of SEQ ID NO: 12, SEQ ID NO: 17 or a sequence that has at least a 98% sequence identity to any thereto; and
(v) a costimulatory signaling domain comprising or consisting of SEQ ID NO: 13 or a sequence that has at least a 98% sequence identity thereto.

In some embodiments, the CAR comprises:
(i) a scFv comprising a VH domain and VL domain, wherein the VH domain comprises a HCDR1 consisting of SEQ ID NO: 1, a HCDR2 consisting of SEQ ID NO: 2 and a HCDR3 consisting of SEQ ID NO: 3; and the VL domain comprises a LCDR1 consisting of SEQ ID NO: 4, a LCDR2 consisting of SEQ ID NO: 5 and a LCDR3 consisting of SEQ ID NO: 6;
(ii) optionally, a hinge or spacer region comprising or consisting of SEQ ID NO: 10 or a sequence that has at least 99% sequence identity thereto;
(iii) a transmembrane domain comprising or consisting of SEQ ID NO: 11 or a sequence that has at least 99% sequence identity thereto;
(iv) an intracellular signaling domain comprising or consisting of SEQ ID NO: 12, SEQ ID NO: 17 or a sequence that has at least 99% sequence identity to any thereto; and
(v) a costimulatory signaling domain comprising or consisting of SEQ ID NO: 13 or a sequence that has at least 99% sequence identity to thereto.

In some embodiments, the CAR comprises:
(i) a scFv comprising a VH domain and VL domain, wherein the VH domain comprises a HCDR1 consisting of SEQ ID NO: 1, a HCDR2 consisting of SEQ ID NO: 2 and a HCDR3 consisting of SEQ ID NO: 3; and the VL domain comprises a LCDR1 consisting of SEQ ID NO: 4, a LCDR2 consisting of SEQ ID NO: 5 and a LCDR3 consisting of SEQ ID NO: 6;
(ii) optionally, a hinge or spacer region comprising SEQ ID NO: 10;
(iii) a transmembrane domain comprising of SEQ ID NO: 11;
(iv) an intracellular signaling domain comprising of SEQ ID NO: 12 or SEQ ID NO: 17; and
(v) a costimulatory signaling domain comprising SEQ ID NO: 13.

In some embodiments, the CAR comprises:
(i) a scFv comprising a VH domain and VL domain, wherein the VH domain comprises a HCDR1 consisting of SEQ ID NO: 1, a HCDR2 consisting of SEQ ID NO: 2 and a HCDR3 consisting of SEQ ID NO: 3; and the VL domain comprises a LCDR1 consisting of SEQ ID NO: 4, a LCDR2 consisting of SEQ ID NO: 5 and a LCDR3 consisting of SEQ ID NO: 6;
(ii) optionally, a hinge or spacer region consisting of SEQ ID NO: 10;
(iii) a transmembrane domain consisting of SEQ ID NO: 11;
(iv) an intracellular signaling domain consisting of SEQ ID NO: 12 or SEQ ID NO: 17; and
(v) a costimulatory signaling domain consisting of SEQ ID NO: 13.

In some embodiments, the CAR comprises:
(i) a scFv comprising a VH domain and VL domain, wherein the VH domain consists of SEQ ID NO: 7 and the VL domain consists of SEQ ID NO: 8;
(ii) optionally, a hinge or spacer region comprising or consisting of SEQ ID NO: 10 or a sequence that has at least 95% sequence identity thereto;
(iii) a transmembrane domain comprising or consisting of SEQ ID NO: 11 or a sequence that has at least 95% sequence identity thereto;
(iv) an intracellular signaling domain comprising or consisting of SEQ ID NO: 12, SEQ ID NO: 17 or a sequence that has at least 95% sequence identity to any thereto; and
(v) a costimulatory signaling domain comprising or consisting of SEQ ID NO: 13 or a sequence that has at least 95% sequence identity thereto.

In some embodiments, the CAR comprises:
(i) a scFv comprising a VH domain and VL domain, wherein the VH domain consists of SEQ ID NO: 7 and the VL domain consists of SEQ ID NO: 8;
(ii) optionally, a hinge or spacer region comprising or consisting of SEQ ID NO: 10 or a sequence that has at least 98% sequence identity thereto;
(iii) a transmembrane domain comprising or consisting of SEQ ID NO: 11 or a sequence that has at least 98% sequence identity thereto;
(iv) an intracellular signaling domain comprising or consisting of SEQ ID NO: 12, SEQ ID NO: 17 or a sequence that has at least 98% sequence identity to any thereto; and
(v) a costimulatory signaling domain comprising or consisting of SEQ ID NO: 13 or a sequence that has at least 98% sequence identity thereto.

In some embodiments, the CAR comprises:
(i) a scFv comprising a VH domain and VL domain, wherein the VH domain consists of SEQ ID NO: 7 and the VL domain consists of SEQ ID NO: 8;
(ii) optionally, a hinge or spacer region comprising or consisting of SEQ ID NO: 10 or a sequence that has at least 99% sequence identity thereto;
(iii) a transmembrane domain comprising or consisting of SEQ ID NO: 11 or a sequence that has at least 99% sequence identity thereto;
(iv) an intracellular signaling domain comprising or consisting of SEQ ID NO: 12, SEQ ID NO: 17 or a sequence that has at least 99% sequence identity to any thereto; and
(v) a costimulatory signaling domain comprising or consisting of SEQ ID NO: 13 or a sequence that has at least 99% sequence identity thereto.

In some embodiments, the CAR comprises:
(i) a scFv comprising a VH domain and VL domain, wherein the VH domain consists of SEQ ID NO: 7 and the VL domain consists of SEQ ID NO: 8;
(ii) optionally, a hinge or spacer region comprising SEQ ID NO: 10;
(iii) a transmembrane domain comprising SEQ ID NO: 11;
(iv) an intracellular signaling domain comprising SEQ ID NO: 12 or SEQ ID NO: 17; and
(v) a costimulatory signaling domain comprising SEQ ID NO: 13.

In some embodiments, the CAR comprises:
(i) a scFv comprising a VH domain and VL domain, wherein the VH domain consists of SEQ ID NO: 7 and the VL domain consists of SEQ ID NO: 8;
(ii) optionally, a hinge or spacer region consisting of SEQ ID NO: 10;
(iii) a transmembrane domain consisting of SEQ ID NO: 11;
(iv) an intracellular signaling domain consisting of SEQ ID NO: 12 or SEQ ID NO: 17; and
(v) a costimulatory signaling domain consisting of SEQ ID NO: 13.

In some embodiments, the CAR comprises:
(i) a scFv comprising or consisting of SEQ ID NO: 9;
(ii) optionally, a hinge or spacer region comprising or consisting of SEQ ID NO: 10 or a sequence that has at least 95% sequence identity thereto;
(iii) a transmembrane domain comprising or consisting of SEQ ID NO: 11 or a sequence that has at least 95% sequence identity thereto;
(iv) an intracellular signaling domain comprising or consisting of SEQ ID NO: 12, SEQ ID NO: 17 or a sequence that has at least 95% sequence identity to any thereto; and
(v) a costimulatory signaling domain comprising or consisting of SEQ ID NO: 13 or a sequence that has at least 95% sequence identity thereto.

In some embodiments, the CAR comprises:
(i) a scFv comprising or consisting of SEQ ID NO: 9;
(ii) optionally, a hinge or spacer region comprising or consisting of SEQ ID NO: 10 or a sequence that has at least 98% sequence identity thereto;
(iii) a transmembrane domain comprising or consisting of SEQ ID NO: 11 or a sequence that has at least 98% sequence identity thereto;
(iv) an intracellular signaling domain comprising or consisting of SEQ ID NO: 12, SEQ ID NO: 17 or a sequence that has at least 98% sequence identity to any thereto; and
(v) a costimulatory signaling domain comprising or consisting of SEQ ID NO: 13 or a sequence that has at least 98% sequence identity thereto.

In some embodiments, the CAR comprises:
(i) a scFv comprising or consisting of SEQ ID NO: 9;
(ii) optionally, a hinge or spacer region comprising or consisting of SEQ ID NO: 10 or a sequence that has at least 99% sequence identity thereto;
(iii) a transmembrane domain comprising or consisting of SEQ ID NO: 11 or a sequence that has at least 99% sequence identity thereto;
(iv) an intracellular signaling domain comprising or consisting of SEQ ID NO: 12, SEQ ID NO: 17 or a sequence that has at least 99% sequence identity to any thereto; and
(v) a costimulatory signaling domain comprising or consisting of SEQ ID NO: 13 or a sequence that has at least 99% sequence identity thereto.

In some embodiments, the CAR comprises:
(i) a scFv comprising or consisting of SEQ ID NO: 9;
(ii) optionally, a hinge or spacer region comprising SEQ ID NO: 10;
(iii) a transmembrane domain comprising SEQ ID NO: 11;
(iv) an intracellular signaling domain comprising SEQ ID NO: 12 or SEQ ID NO: 17; and
(v) a costimulatory signaling domain comprising SEQ ID NO: 13.

In some embodiments, the CAR comprises:
(i) a scFv consisting of SEQ ID NO: 9;
(ii) optionally, a hinge or spacer region consisting of SEQ ID NO: 10;
(iii) a transmembrane domain consisting of SEQ ID NO: 11;
(iv) an intracellular signaling domain consisting of SEQ ID NO: 12 or SEQ ID NO: 17; and
(v) a costimulatory signaling domain consisting of SEQ ID NO: 13.

Preferably, the intracellular signaling domain in (iv) of any of the above embodiments is SEQ ID NO: 17.

In some embodiments, the CAR comprises or consists of SEQ ID NO: 14 or a sequence that has at least 95% sequence identity thereto. In some embodiments, the CAR comprises or consists of SEQ ID NO: 14 or a sequence that has at least 98% sequence identity thereto. In some embodiments, the CAR comprises or consists of SEQ ID NO: 14 or a sequence that has at least 99% sequence identity thereto. In some preferred embodiments, the CAR comprises or consists of SEQ ID NO: 14.

Full sequence of the CAR (SEQ ID NO: 14) without signaling
peptide- CD3ζ intracellular domain variant

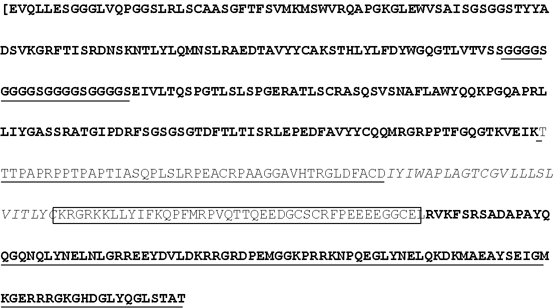

Legend: F8-ScFv in bold (VH-linker-VL); hinge CD8 underlined;
CD8 transmembrane (italics), 41BB co-stimulatory domain
(square); CD3ζ intracellular domain (bold and underlined).
Full sequence of the CAR (SEQ ID NO: 18) without signaling
peptide - complete CD3ζ intracellular domain

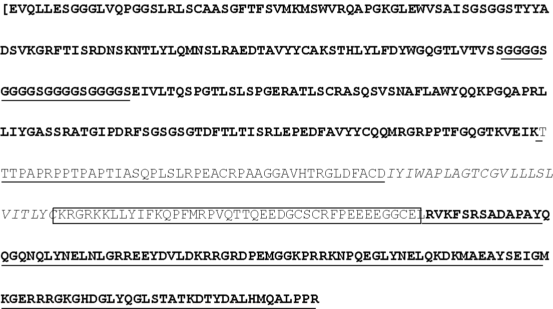

Legend: F8-ScFv in bold (VH-linker-VL); hinge CD8 underlined;
CD8 transmembrane (italics), 41BB co-stimulatory domain
(square); CD3ζ intracellular domain (bold and underlined).

In these particular embodiments, the CAR comprises the following elements in the indicated order:
EDA-ScFv-HingeCD8-CD8 Transmembrane-41bb (CD137)-CD3z

The polynucleotide coding for the EDA-targeting moiety, the hinge or spacer region, the transmembrane domain, the intracellular signaling domain, the costimulatory signaling domain and the CAR polypeptide can be readily prepared by synthetic or recombinant methods from the amino acid sequence. In some embodiments, the coding polynucleotide of the EDA-targeting moiety is operably linked to a transmembrane region coding polynucleotide which is operably linked to a polynucleotide coding for an intracellular signaling domain. In some embodiments, the coding polynucleotide of the EDA-targeting moiety is operably linked to a hinge or spacer region coding polynucleotide which is operably linked to a transmembrane region coding polynucleotide which is operably linked to a polynucleotide coding for the intracellular signaling domain. In some embodiments, the coding polynucleotide of the EDA-targeting moiety is operably linked to a hinge or spacer region coding polynucleotide which is operably linked to a transmembrane region coding polynucleotide which is operably linked to a polynucleotide coding for a costimulatory signaling domain which is operably linked to a intracellular signaling domain coding polynucleotide. In some embodiments, the coding polynucleotide of the EDA-targeting moiety is operably linked to a hinge or spacer region coding polynucleotide which is operably linked to a transmembrane region coding polynucleotide which is operably linked to a polynucleotide coding for the intracellular signaling domain which is operably linked to a costimulatory signaling domain coding polynucleotide. In some embodiments, the polynucleotide coding for any of the above may also have one or more restriction enzyme sites at the 5′ and/or 3′ ends of the coding sequence to be excised and replaced by other components for customization of the CAR polypeptide. Genetic engineering techniques are well known in the art and are described in handbooks such as Sambrook et al, “Molecular Cloning: A Laboratory Manual” (4th.Ed.), Vols. 1-3, Cold Spring Harbor Laboratory Press (2012).

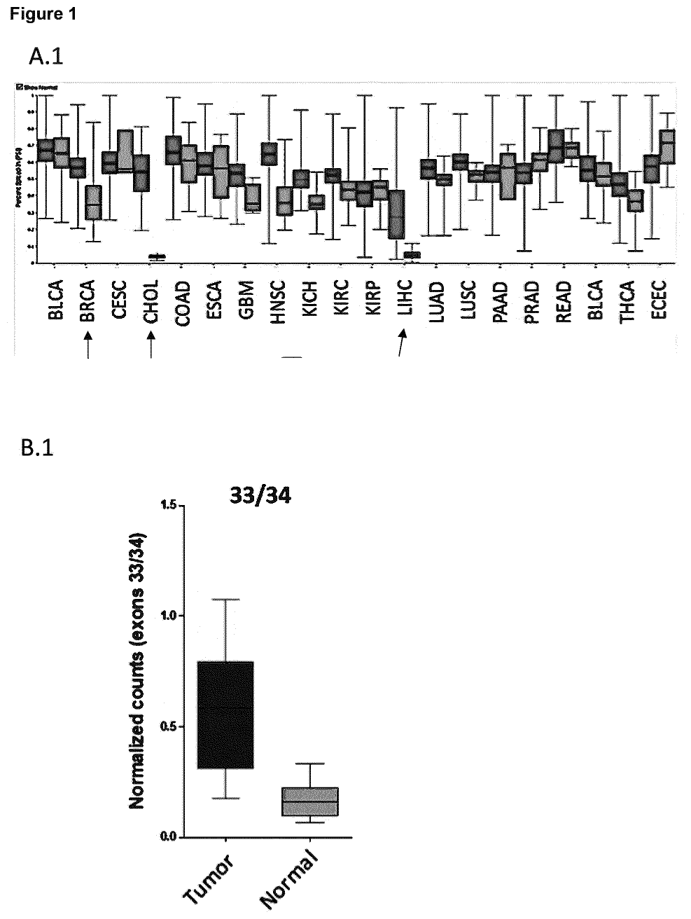

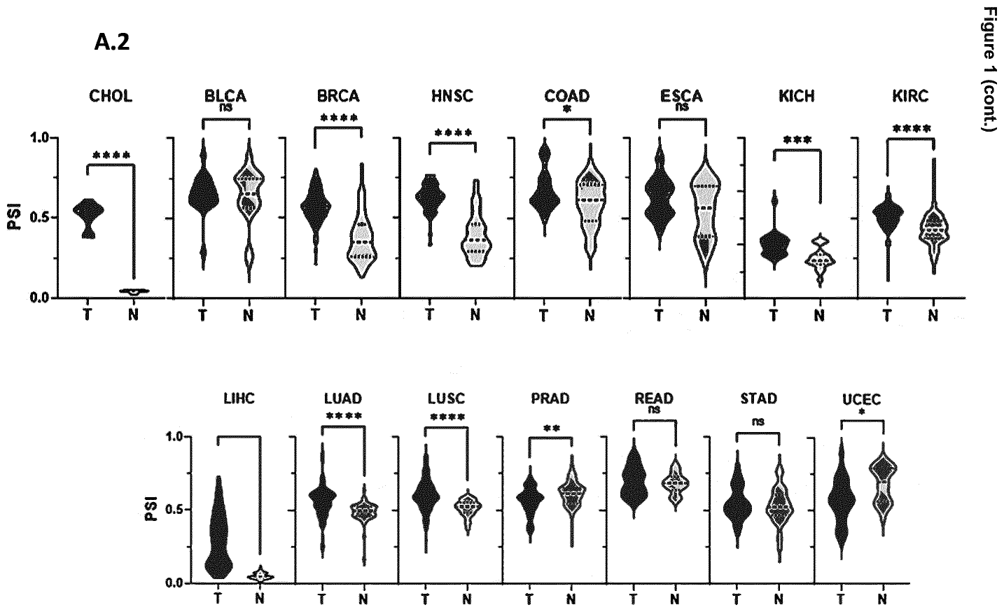

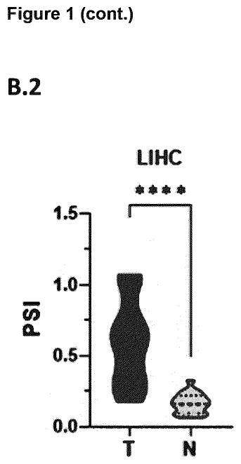

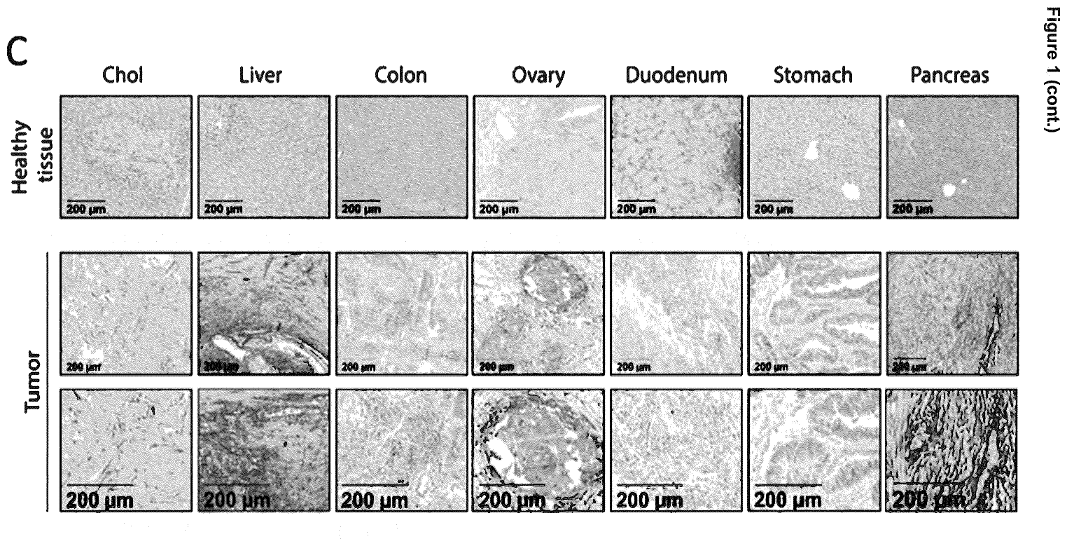

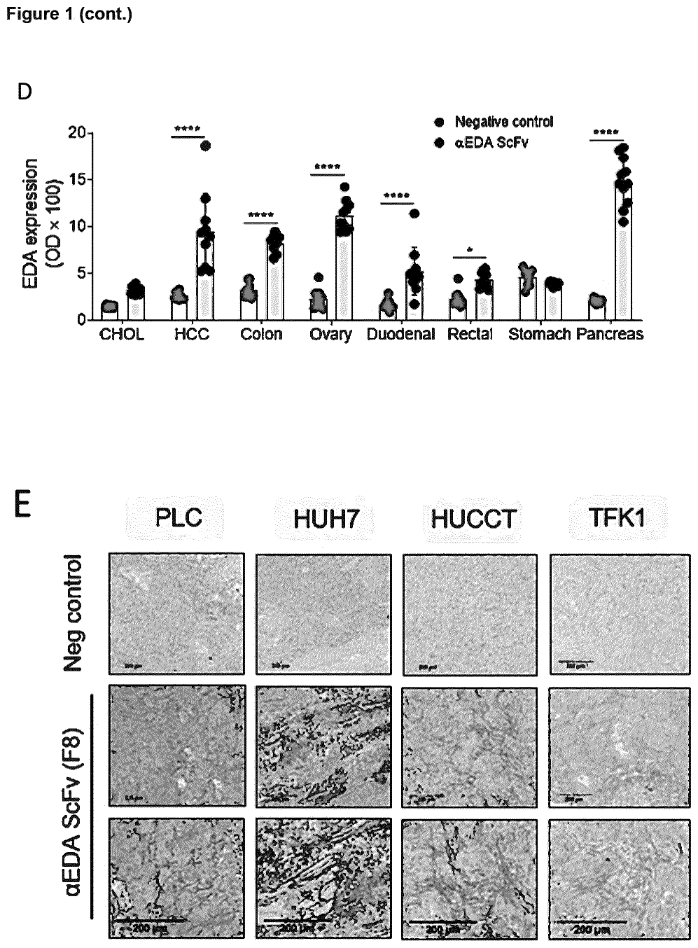

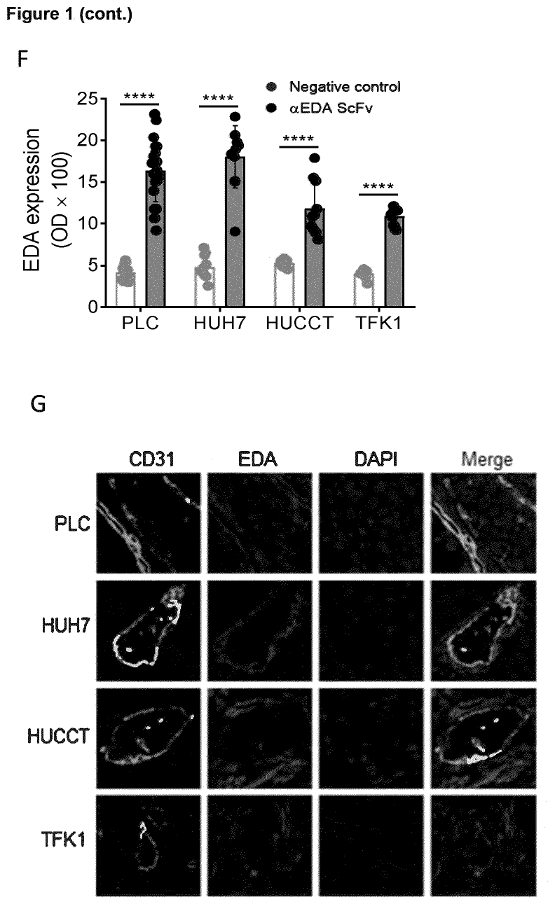

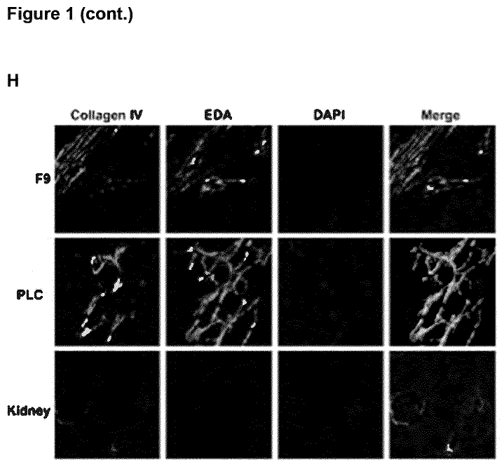

In some embodiments, said nucleic acid is operably linked to at least one promoter, preferably to a eukaryotic promoter, i.e., which enables the expression of the inserted coding sequence in eukaryotic cells, for instance, in mammalian cells. In some embodiments, optionally in combination with any of the above, to favor protein expression on the cell membrane, the CAR polynucleotide has a signal peptide at is N-terminal end. In these embodiments, the polynucleotide encoding the signal peptide will be present in the CAR encoding polynucleotide, but the encoded peptide will not be present in the polypeptide expressed in the cell membrane. Preferably, said signal peptide is useful for the secretion of a recombinant polypeptide from a mammalian cell. Signal peptides from human proteins can be obtained for instance from the Signal Peptide Database (http://www.signalpeptide.de). Some examples of human signal peptides of 16 amino acids in length are provided at S1 FIG. 1 of Güler-Gane et al. 2016. Some other example signal peptides include the signal peptides of secrecon, mouse IgKVIII, human IgKVIII, CD33, tPA as disclosed in Table 1 of of Güler-Gane et al. 2016.

In some embodiments, the signal peptide comprises or consists of the signal peptide of the transmembrane domain used. In some embodiments, the signal peptide comprises or consists of the CD8 signal peptide (SEQ ID NO: 15) or a sequence that has at least 95% sequence identity thereto. In some embodiments, the signal peptide comprises or consists of SEQ ID NO: 15 or a sequence that has at least 98% sequence identity thereto. In some embodiments, the signal peptide comprises or consists of SEQ ID NO: 15 or a sequence that has at least 99% sequence identity thereto. In some preferred embodiments, the signal peptide comprises or consists of SEQ ID NO: 15.

Signal peptide
(SEQ ID NO: 15)
MALPVTALLLPLALLLHAARP

In some embodiments, the CAR polynucleotide of the invention comprises a polynucleotide encoding the CD8 signal peptide at his N-terminal end. In preferred embodiments, said polynucleotide encodes an EDA-CAR which comprises or consists of the following elements as described herein above:
CD8 signal peptide—EDA ScFv (F8) Heavy chain-Linker-EDA scFv (F8) Light chain—CD8a hinge and transmembrane sequence—CDF3z chain.

In a particular embodiment, the CAR polynucleotide of the invention encodes a human EDA CAR polypeptide of SEQ ID NO: 16:

MALPVTALLLPLALLLHAARPEVQLLESGGGLVQPGGSLRLSCAASGFT
FSVMKMSWVRQAPGKGLEWVSAISGSGGSTYYADSVKGRFTISRDNSKN
TLYLQMNSLRAEDTAVYYCAKSTHLYLFDYWGQGTLVTVSSGGGGSGGG
GSGGGGSGGGGSEIVLTQSPGTLSLSPGERATLSCRASQSVSNAFLAWY
QQKPGQAPRLLIYGASSRATGIPDRFSGSGSGTDFTLTISRLEPEDFAV
YYCQQMRGRPPTFGQGTKVEIKTTTPAPRPPTPAPTIASQPLSLRPEAC
RPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYQ
QGQNQLYNELNLGRREEYDVLDKRRGRDPEMGGKPRRKNPQEGLYNELQ
KDKMAEAYSEIGMKGERRRGKGHDGLYQGLSTAT

SEQ ID NO: 16 comprises SEQ ID NO: 12 as CD34 intracellular domain.

In another embodiment, the CAR polynucleotide of the invention encodes a human EDA CAR polypeptide of SEQ ID NO: 19:

MALPVTALLLPLALLLHAARPEVQLLESGGGLVQPGGSLRLSCAASGFT
FSVMKMSWVRQAPGKGLEWVSAISGSGGSTYYADSVKGRFTISRDNSKN
TLYLQMNSLRAEDTAVYYCAKSTHLYLFDYWGQGTLVTVSSGGGGSGGG
GSGGGGGGGGSEIVLTQSPGTLSLSPGERATLSCRASQSVSNAFLAWYQ
QKPGQAPRLLIYGASSRATGIPDRFSGSGSGTDFTLTISRLEPEDFAVY
YCQQMRGRPPTFGQGTKVEIKTTTPAPRPPTPAPTIASQPLSLRPEACR
PAAGGAVHTRGLDFACDIYIWAPLAGTCGVLLLSLVITLYCKRGRKKLL
YIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR

SEQ ID NO: 19 comprises SEQ ID NO: 17 as CD37 intracellular domain.

Annotation of the human CAR is as provided above for SEQ ID NO: 14 and SEQ ID NO: 18, respectively. The signal peptide added at the N-terminal end is highlighted in bold letters.

In a preferred embodiment, said polynucleotide encoding the human EDA CAR consisting of SEQ ID NO: 19 is SEQ ID NO: 20:

atggccctgcctgtgaccgccctgctgctgcccctggccctgctgctgc
acgcggcgcgcccgGAGGTTCAGTTGTTGGAGAGCGGCGGTGGACTCGT
TCAGCCTGGTGGTTCATTGCGGCTTTCATGCGCTGCAAGTGGATTCACC
ttcAGCGTTATGAAGATGTCTTGGGTCCGACAAGCTCCAGGGAAGGGAC
TTGAGTGGGTGTCCGCCATCTCTGGCTCAGGCGGCAGTACCTATTATGC
CGACTCAGTGAAGGGGAGATTTACTATCTCTAGAGACAATTCTAAGAAT
ACCCTCTATCTCCAGATGAACTCACTGCGCGCTGAAGACACCGCCGTGT
ATTACTGTGCAAAAAGCACCCATCTCTACCTGTTTGATTACTGGGGACA
AGGCACACTTGTGACAGTGAGTTCTGGCGGCGGTGGTTCCGGGGGTGGA
GGCTCCGGAGGTGGAGGTAGCGGCGGGGGTGGTTCTGAAATAGTACTCA
CTCAAAGCCCAGGAACCCTCTCTCTTAGCCCAGGTGAGAGGGCCACTCT
CTCCTGTCGAGCATCTCAGTCCGTCTCTAACGCTTTCCTGGCATGGTAC
CAGCAAAAACCCGGCCAAGCACCACGACTTCTCATCTACGGTGCTAGTA
GCAGAGCTACCGGTATACCCGACCGATTCTCAGGTTCAGGAAGTGGAAC
AGACTTTACATTGACCATTTCCCGACTGGAACCCGAGGACTTCGCTGTA
TATTACTGCCAGCAGATGCGCGGACGGCCCCCCACTTTCGGCCAAGGCA
CTAAAGTAGAGATCAAGaccacaacccctgcccctaggccaccaacacc
agcacctaccatcGCTAGCcagccactgagcctgcggcccgaggcctgt
aggccagccgccggcggagcagtgcacacccggggcctggactttgcct
gcgatatctacatctgggcaccactggccggaacatgtggcgtgctgct
gctgagcctggtcatcaccctgtattgcaagagaggcaggaagaagctg
ctgtacatcttcaagcagcccttcatgcggcccgtgcagacaacccagg
aggaggacggctgcagctgtaggttccctgaggaggaggagggaggatg
tgagctgcgcgtgaagttttctcggagcgccgatgcaccagcatatcag
cagggacagaaccagctgtacaacgagctgaatctgggccggagagagg
agtatgacgtgctggataagaggcgcggcagggaccctgagatgggcgg
caagcctagaagaaagaacccccaggaaggcctgtataacgaactgcag
aaagacaagatggccgaggcctacagcgagatcggaatgaagggcgagc
ggagaagaggcaagggccacgatggcctgtaccagggactgagcaccgc
caccaaggatacctatgacgcactgcacatgcaggccctgccccccaga
TGA

In a particular embodiment, the CAR polynucleotide of the invention encodes a murine EDA CAR polypeptide comprising or consisting of SEQ ID NO: 21:

MASPLTRFLSLNLLLLGESIILGSGEAEVQLLESGGGLVQPGGSLRLSCAASGFTFSVMKMSWVRQAP
GKGLEWVSAISGSGGSTYYADSVKGRFTISRDNSKNTLYLQMNSLRAEDTAVYYCAKSTHLYLFDYWG
QGTLVTVSSGGGGSGGGGSGGGGSGGGGSEIVLTQSPGTLSLSPGERATLSCRASQSVSNAFLAWY
QQKPGQAPRLLIYGASSRATGIPDRFSGSGSGTDFTLTISRLEPEDFAVYYCQQMRGRPPTFGQGTKV
EIKQLTTTKPVLRTPSPVHPTGTSQPQRPEDCRPRGSVKGTGLDFACDIYIWAPLAGICVALLLSLIITLICY

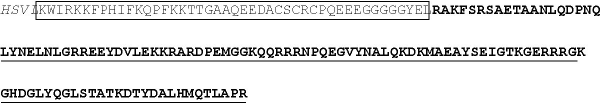

Legend: signal peptide CD8 in bold, italics and underlined; F8-ScFv
in bold (VH-linker-VL); hinge CD8 underlined; CD8 transmembrane
(italics), 41BB co-stimulatory domain (square); CD3ζ intracellular
domain (bold and underlined).

In a preferred embodiment, said polynucleotide encoding the murine EDA CAR consisting of SEQ ID NO: 21 is SEQ ID NO: 22:

ATGGCAAGTCCTCTGACAAGGTTTCTCTCCCTCAATCTCCTCCTCCTGG
GCGAGTCTATCATTCTGGGTAGCGGTGAAGCCGAGGTTCAGTTGTTGGA
GAGCGGCGGTGGACTCGTTCAGCCTGGTGGTTCATTGCGGCTTTCATGC
GCTGCAAGTGGATTCACCttcAGCGTTATGAAGATGTCTTGGGTCCGAC
AAGCTCCAGGGAAGGGACTTGAGTGGGTGTCCGCCATCTCTGGCTCAGG
CGGCAGTACCTATTATGCCGACTCAGTGAAGGGGAGATTTACTATCTCT
AGAGACAATTCTAAGAATACCCTCTATCTCCAGATGAACTCACTGCGCG
CTGAAGACACCGCCGTGTATTACTGTGCAAAAAGCACCCATCTCTACCT
GTTTGATTACTGGGGACAAGGCACACTTGTGACAGTGAGTTCTGGCGGC
GGTGGTTCCGGGGGTGGAGGCTCCGGAGGTGGAGGTAGCGGCGGGGGTG
GTTCTGAAATAGTACTCACTCAAAGCCCAGGAACCCTCTCTCTTAGCCC
AGGTGAGAGGGCCACTCTCTCCTGTCGAGCATCTCAGTCCGTCTCTAAC
GCTTTCCTGGCATGGTACCAGCAAAAACCCGGCCAAGCACCACGACTTC
TCATCTACGGTGCTAGTAGCAGAGCTACCGGTATACCCGACCGATTCTC
AGGTTCAGGAAGTGGAACAGACTTTACATTGACCATTTCCCGACTGGAA
CCCGAGGACTTCGCTGTATATTACTGCCAGCAGATGCGCGGACGGCCCC
CCACTTTCGGCCAAGGCACTAAAGTAGAGATCAAGCAATTGACCACAAC
CAAGCCAGTGCTTCGAACCCCATCCCCTGTGCACCCAACAGGAACCTCT
CAGCCACAGAGGCCCGAGGACTGTAGGCCAAGAGGCTCCGTGAAGGGAA
CAGGCCTGGACTTTGCCTGTGATATCTACATCTGGGCTCCCCTGGCTGG
AATCTGCGTGGCTCTGCTGCTGAGCCTGATCATCACCCTGATCTGCTAC
CACTCCGTGCTGAAGTGGATCAGGAAGAAGTTCCCCCACATCTTTAAGC
AGCCTTTCAAGAAGACAACCGGAGCTGCTCAGGAGGAGGACGCTTGCTC
TTGTCGCTGCCCTCAGGAAGAAGAAGGAGGAGGAGGAGGATACGAGCTG
AGGGCCAAGTTCAGCAGATCCGCTGAGACAGCCGCTAACCTGCAGGACC
CCAACCAGCTGTACAACGAGCTGAACCTGGGCAGGAGAGAGGAGTACGA
CGTGCTGGAGAAGAAGAGGGCCAGGGACCCCGAGATGGGAGGAAAGCAG
CAGCGGCGCAGGAACCCTCAGGAGGGCGTGTACAACGCTCTGCAGAAGG
ACAAGATGGCCGAGGCTTACAGCGAGATCGGAACCAAGGGAGAGAGACG
GCGCGGAAAGGGACACGATGGACTGTACCAGGGCCTGAGCACAGCAACT
AAAGACACTTACGATGCCCTCCACATGCAGACCCTCGCCCCACGGTGA

In some embodiments, the polynucleotide is suitable for transducing or transforming a cell. In some embodiments, the polynucleotide is suitable for transducing or transforming a T cell for use in adoptive immunotherapy. In some embodiments, the polynucleotide is codon optimized for expression in mammalian cells. Codon optimization methods are known in the art (see, for example, Parret et al., 2016. Curr Opin Struct Biol. 39:155-162).

In a second aspect the invention refers to a chimeric antigen receptor (CAR) polypeptide—also referred herein as chimeric receptor polypeptide-, coded for by a chimeric receptor nucleic acid as described herein. It can be characterized by its domain composition and/or protein sequence which may be as described herein above. In preferred embodiments, the CAR polypeptide of the invention comprises or consists of SEQ ID NO: 14 or SEQ ID NO: 18, preferably comprises or consists of SEQ ID NO: 18.

### Expression Vector and Host Cells of the Invention

In a third aspect the invention refers to an expression vector comprising a chimeric receptor nucleic acid as described herein. By “vector” is meant any genetic element, such as a plasmid, phage, transposon, cosmid, chromosome, virus, virion, etc., which is capable of replication when associated with the proper control elements and which can transfer gene sequences between cells. Expression vector also refers to a nucleic acid molecule capable of effecting expression of a gene (transgene) in host cells or host organisms compatible with such sequences. Expression vectors typically include at least suitable transcription regulatory sequences and optionally, 3′ transcription termination signals. Additional factors necessary or helpful in effecting expression may also be present, such as expression enhancer elements able to respond to a precise inductive signal (endogenous or chimeric transcription factors) or specific for certain cells, organs or tissues. Vectors include, but are not limited to, plasmids, phasmids, cosmids, transposable elements, viruses, and artificial chromosomes (e.g., YACs). Preferably, the vector of the invention is a vector suitable for use in gene or cell therapy.

In some embodiments, the expression vector is a non-viral vector, such as a plasmid, phasmid, cosmid, transposon-based vector, and artificial chromosomes (e.g., YACs). In other embodiments, the expression vector is a viral vector, such as vectors derived from Moloney murine leukemia virus vectors (MoMLV), MSCV, SFFV, MPSV or SNV, lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors. As is known in the art, depending on the specific viral vector considered, suitable sequences should be introduced in the vector of the invention for obtaining a functional viral vector, such as AAV ITRs for an AAV vector, or LTRs for retroviral or lentiviral vectors.

In some embodiments, the polynucleotide of the present invention may be comprised in a γ-retroviral or lentiviral vector which can be used to transduce or transform a T cell (see Rivière & Sadelain, 2017). The nucleic acid may also be inserted into a cell through the use of DNA transposons, RNA transfection or genome editing techniques such as TALEN, ZFN and CRISPR/Cas9 (see Rivière & Sadelain, 2017).

Retroviruses belong to the family Retroviridae, which is composed of numerous non-icosahedral, enveloped viruses which possess two copies of a single-stranded RNA genome that has a short dimerized region. Retroviruses are a common tool for gene delivery. Once the virus is integrated into the host genome, it is referred to as a “provirus.” The provirus serves as a template for RNA polymerase II and directs the expression of RNA molecules which encode the structural proteins and enzymes needed to produce new viral particles. Illustrative retroviruses include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV)) and lentivirus. In some embodiments, said retroviral vector is a γ-retroviral vector. Gamma-retroviral vectors have been widely used to generate CAR T cells for both preclinical and clinical settings (Watanabe N. and Mckenna M K, 2022). Gamma-retroviral vectors have a relatively high transduction efficiency, but in contrast to lentiviral vectors, their infectivity is limited to dividing cells. In some embodiments, said viral vector comprises the LTR of the Moloney murine Leukemia Virus (MLV), such as the pCL vector system (Naviaux R K et al. 1996).

As used herein, the term “lentivirus” refers to a group of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

A lentivirus usually comprises three (2nd generation) or four (3^{rd} generation) expression systems, comprising the following elements:
Lentiviral transfer plasmid encoding the insert of interest (e.g. the CAR as described herein). The transgene sequence is flanked by long terminal repeat (LTR) sequences, which facilitate integration of the transfer plasmid sequences into the host genome. Typically, it is the sequences between and including the LTRs that is integrated into the host genome upon viral transduction. For safety reasons, transfer plasmids are all replication incompetent and may contain an additional deletion in the 3′LTR, rendering the virus “self-inactivating” (SIN) after integration.
Packaging plasmid(s): Comprises the elements required for virus packaging, such as genes coding for structural proteins, other genes (except the Env gene). This plasmid lacks the packaging signal owing to which the virus is rendered incapable of reproduction after it has infected host cell.
Envelope plasmid: Encodes the viral envelope proteins.

The nucleic acid construct or the expression vector of the invention may be packaged into a virus capsid to generate a “viral particle”, also named “viral vector particle”. Thus, in an aspect the present invention relates to a viral particle comprising a nucleic acid construct or an expression vector of the invention.

In a forth aspect, the present invention provides a cell comprising the nucleic acid, a vector and/or the CAR polypeptide of the present invention. The EDA CAR cells of the invention can be obtained by any known method of ex vivo gene transfer, such as transduction by using viral vectors as described herein above or transformation using non-viral systems such as electroporation, lipofection, ultrasound, or magnetofection. In another aspect, the invention relates to a method for obtaining the EDA CAR cells of the invention comprising a step of transforming a cell as described herein with a nucleic acid or vector of the invention as described herein.

In some embodiments, the cell is a Natural Killer (NK) cell. In other embodiments, the cell is a T-cell (referred to as CAR T-cell or CART). CD8+ cells and CD4+ cells can be obtained and further sorted into subsets by using standard methods well known in the art.

In some embodiments, the cell is a CD8+T lymphocyte cell selected from the group consisting of naïve CD8+ T cells, stem cell memory CD8+ T cells, central memory CD8+ T cells, effector memory CD8+ T cells and bulk CD8+ T cells.

In other embodiments, the cell is a CD4+T lymphocyte cell selected from the group consisting of naïve CD4+ T cells, stem cell memory CD4+ T cells, central memory CD4+ T cells, effector memory CD4+ T cells and bulk CD4+ T cells.

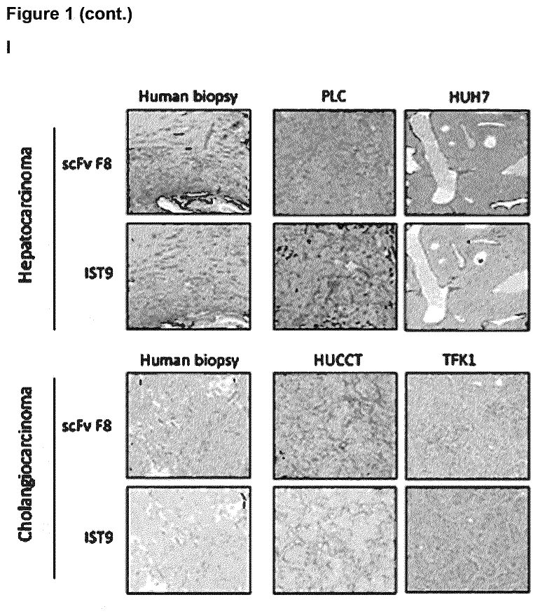

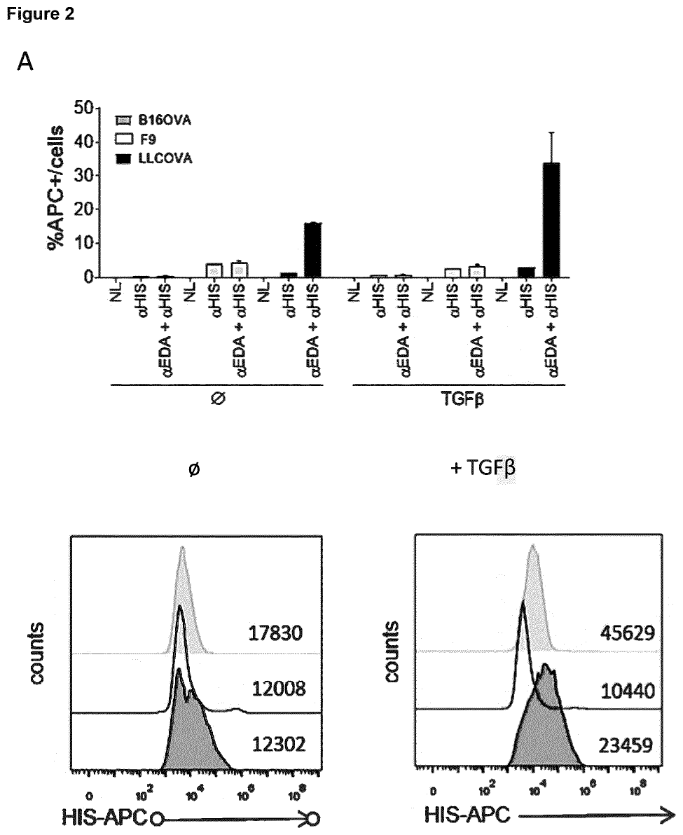

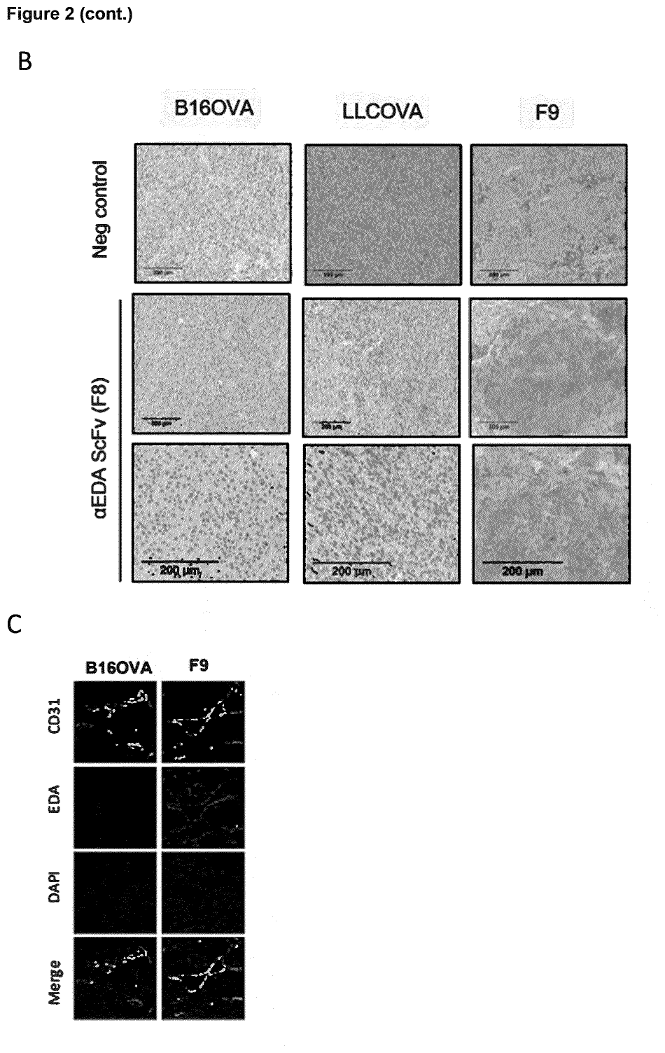

A straightforward and functionally significant means of classifying T cell subsets can be accomplished by assessing for the co-expression of the lymphoid homing molecules L-selectin (CD62L) and CC-chemokine receptor 7 (CCR7). T cells which display these 2 molecules have a propensity to home to secondary lymphoid structures where they can actively survey professional antigen presenting cells for the presence of the corresponding antigen. Cells in this category include naïve T cells (T_{N}) as well as two antigen experienced memory T cell populations: T memory stem cell (T_{SCM}) population and central memory T cells (T_{CM}). Although in humans both T_{N} and T_{SCM} express the RA isoform of CD45, T_{SCM} can be distinguished from T_{N} based on the expression of the IL-2/IL-15β chain receptor (CD122) and Fas (CD95). Central memory T cells, on the other hand, have acquired the expression of the prototypical human antigen experienced T cell marker, CD45RO (Klebanoff C A et al., 2012). For further details on the phenotypes of the indicated cell subsets, see Gattinoni et al. 2017, see FIG. 2.

In some embodiments, the CD4+ or CD8+ cells can be sorted into naïve, stem cell memory, central memory, and effector memory cells by identifying cell surface antigens that are associated with each of those types of cells. In some embodiments, memory T cells are present in both CD62L+ and CD62L− subsets of CD4+ or CD8+ peripheral blood lymphocytes. In some embodiments, the expression of phenotypic markers of central memory include CD45RO, CD62L, CCR7, CD28, CD3, and CD127 and are negative or low for granzyme B. In some embodiments, central memory T cells are CD45RO+, CD62L+, T cells. In some embodiments, effector memory cells are negative for CD62L, CCR7, CD28, and CD127, and positive for granzyme B and perforin. In some embodiments, naïve T lymphocytes are characterized by the expression of phenotypic markers of naïve T cells including CD62L, CCR7, CD28, CD3, CD127, and CD45RA.

In other embodiments, the cell is a T reg lymphocyte. In some embodiments, the Treg lymphocytes are characterized by the expression of phenotypic markers of Treg cells including CD25 and Foxp3. In some embodiments, the Treg cells are characterized by the further expression of functional markers such as CTLA-4 or CD39. The Treg cells of the invention can also be CD4+ or CD8+ Treg cells.

A cell or cell population can be determined to be positive or negative for a particular cell surface marker by flow cytometry using staining with a specific antibody for the surface marker and an isotype matched control antibody. A cell population negative for a marker may refer to the absence of significant staining of the cell population with the antibody specific for the marker above the isotype control, positive may refer to uniform staining of the cell population above the isotype control. In some embodiments, a decrease in expression of one or markers refers to loss of 1 log 10 in the mean fluorescence intensity and/or decrease of percentage of cells that exhibit the marker of at least about 20%, 25%, 30%, 35%, 40%, 45%, 50%, 55%, 60%, 65, 70%, 75%, 80%, 85%, 90%, 95%, or 100% of the cells and any percentage between 20% and 100% when compared to a reference cell population. In some embodiments, a cell population positive for one or more markers may refer to a percentage of cells that exhibit the marker of at least about 50%, 55%, 60%, 65%, 70%, 75%, 80%, 85%, 90%, 95% or 100% of the cells and any percentage between 50% and 100% when compared to a reference cell population.

In some embodiments, the cell is a lymphoid precursor, embryonic stem cell or an induced pluripotent stem cell with the capacity to differentiate into a mature T cell (Rivière & Sadelain, 2017).

In some embodiments, the cell is an autologous cell, such as an autologous T cell. The term “autologous cell” refers to a cell obtained from the same patient that is to be treated using any one of the methods of the present invention.

In some embodiments, the cell is allogeneic, such as an allogeneic T cell. In some embodiments, the cell is an allo-tolerant cell, such as an allo-tolerant T cell. The term “allo-tolerant cell” refers to a cell that has been engineered to decrease the risk of a Graft-versus-host disease response. In some embodiments, this is achieved by genomic editing-mediated deletion of TCR and/or β2-microglobulin. Allo-tolerant cells are known in the art (see section of allogeneic T cells in Rivière & Sadelain, 2017).

In some embodiments, the EDA CAR cells as described herein express an EDA-reactive chimeric receptor and elicit a cellular immune response.

### Pharmaceutical Composition of the Invention

In a fifth aspect, the present invention provides a pharmaceutical composition comprising a host cell, such as a plurality of cells, of the present invention and a pharmaceutically acceptable excipient, carrier or diluent. In some embodiments, said composition is an adoptive cellular immunotherapy composition.

A pharmaceutical composition as described herein may also contain other substances. These substances include, but are not limited to, cryoprotectants, surfactants, anti-oxidants, and stabilizing agents. The term “cryoprotectant” as used herein, includes agents which provide stability to the CARTs against freezing-induced stresses. Non-limiting examples of cryoprotectants include sugars, such as sucrose, glucose, trehalose, mannitol, mannose, and lactose; polymers, such as dextran, hydroxyethyl starch and polyethylene glycol; surfactants, such as polysorbates (e.g., PS-20 or PS-80); and amino acids, such as glycine, arginine, leucine, and serine. A cryoprotectant exhibiting low toxicity in biological systems is generally used.

In some embodiments, the cells are formulated by first harvesting them from their culture medium, and then washing and concentrating the cells in a medium and container system suitable for administration (a “pharmaceutically acceptable” carrier) in a therapeutically effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin, fetal bovine serum or other human serum components.

In some embodiments, the pharmaceutical composition comprises:
a CD8+T lymphocyte cell of the invention;
a CD4+T lymphocyte cell of the invention;
a CD8+T lymphocyte cell of the invention and a CD4+T lymphocyte cell of the invention;
a CD8+T lymphocyte cell of the invention and not a CD4+T lymphocyte cell of the invention; or
a CD4+T lymphocyte cell of the invention and not a CD8+T lymphocyte cell of the invention.

In some embodiments, it comprises a CD8+T lymphocyte cell of the invention and a CD4+T lymphocyte cell of the invention. The CD8+: CD4+ ratio in number of cells can be from 1:10 to 10:1, preferably from 1:9 to 9:1, from 1:8 to 8:1, from 1:7 to 7:1, from 1:6 to 6:1, from 1:5 to 5:1, from 1:4 to 4:1, from 1:3 to 3:1 or from 1:2 to 2:1. In some embodiments, the CD8+: CD4+ ratio in number of cells is of 1:1. In other embodiments, the CD8+: CD4+ ratio is 6:2, 6:2.5, 2:7 or 2:10.

In some embodiments, the pharmaceutical composition comprises a CD4+T lymphocyte cell of the invention and not a CD8+T lymphocyte cell of the invention.

A pharmaceutical composition is typically formulated to be compatible with its intended route of administration. Methods to accomplish the administration are known to those of ordinary skill in the art. These includes, for example, injections, by parenteral routes such as intravenous, intravascular, intraarterial, subcutaneous, intramuscular, intraperitoneal, intraventricular, intraepidural, intratumoral, intranodal or others; as well as oral, nasal, ophthalmic, rectal or topical administration. Sustained release administration is also specifically contemplated, e.g., as depot injections or erodible implants. Localized delivery is particularly contemplated, e.g., as delivery via a catheter to one or more arteries, such as the renal artery or a vessel supplying a localized site of interest. In one embodiment, said pharmaceutical composition is for oral administration. In another embodiment, said pharmaceutical composition is for intravenous, intramuscular or subcutaneous infusion or injection.

Said pharmaceutical composition can be administered a single time. It may also be administered regularly throughout the course of the method of treatment, for example, one, two, three, four, or more times a day, weekly, bi-weekly, every three weeks or monthly. The pharmaceutical composition may also be administered continuously to the subject (e.g, intravenously or by release from an implant, pump, sustained release formulation, etc.). The dosage to be administered can depend on multiple factors, including the type and severity of the cancer and/or on the characteristics of the subject, such as general health, age, sex, body weight and tolerance to drugs and should be adjusted, as needed, according to individual need and professional judgment. The dosage may also vary depending upon factors, such as route of administration, target site, or other therapies administered. The skilled artisan will be able to determine appropriate doses depending on these and other factors. A therapeutically effective amount may include, but is not limited to, dosage ranges of about 1×10⁴ cells/kg to about 1×10⁹ cells/kg, about 1×10⁵ cells/kg to about 1×10⁸ cells/kg or preferably about 1×10⁶ cells/kg to about 1×10⁷ cells/kg. The indicated dosage ranges are preferably doses per infusion.

Therapeutic agents and methods of administration, dosages, etc., are well known to those of skill in the art (see for example, the “Physicians Desk Reference”, Klaassen's “The Pharmacological Basis of Therapeutics”, “Remington's Pharmaceutical Sciences”, and “The Merck Index, Eleventh Edition”, incorporated herein by reference), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

### Medical Uses and Kit of the Invention

In a sixth aspect, the present invention provides a chimeric receptor nucleic acid comprising:
a) a polynucleotide coding for a ligand binding domain comprising an extra-domain A of fibronectin (EDA) targeting-moiety as described herein, preferably a single chain Fv (scFv);
b) optionally, a polynucleotide coding for a hinge or spacer region;
c) a polynucleotide coding for a transmembrane domain as described herein; and
d) a polynucleotide coding for an intracellular signaling domain as described herein;
   or a chimeric receptor polypeptide coded for thereby as described herein;
   an expression vector comprising the chimeric receptor nucleic acid as described herein;
   a host cell comprising the chimeric receptor nucleic acid or the expression vector as described herein; or
   a pharmaceutical composition comprising any thereof and further comprising and a pharmaceutically acceptable excipient, diluent or carrier as described herein for use as a medicament. Preferably, it refers to a cell according to the present invention or a pharmaceutical composition according to the present invention for use as a medicament.

### EDA-CAR Cells in the Treatment of EDA-Positive Cancer

In a seventh aspect, the present invention provides a chimeric receptor nucleic acid comprising:
a) a polynucleotide coding for a ligand binding domain comprising an extra-domain A of fibronectin (EDA) targeting-moiety as described herein, preferably a single chain Fv (scFv);
b) optionally, a polynucleotide coding for a hinge or spacer region;
c) a polynucleotide coding for a transmembrane domain; and
d) a polynucleotide coding for an intracellular signaling domain; or
   a chimeric receptor polypeptide coded for thereby as described herein;
   an expression vector comprising the chimeric receptor nucleic acid as described herein;
   a host cell comprising the chimeric receptor nucleic acid or the expression vector as described herein; or
   a pharmaceutical composition comprising any thereof and further comprising and a pharmaceutically acceptable excipient, diluent or carrier as described herein for use in a method of treating an EDA-positive cancer. Preferably, the cell of the present invention or the pharmaceutical composition of the present invention for use in a method of treating an EDA-positive cancer in a subject.

In a related aspect the present invention refers to a method for the treatment of an EDA-positive cancer in a subject, said method comprising administering the polynucleotide coding for a CAR, a CAR polypeptide, a vector, a host cell or a pharmaceutical composition as described herein to a subject in need thereof. Preferably, wherein said method comprises administering the cell of the present invention or the pharmaceutical composition of the present invention to a subject in need thereof.

In a further related aspect, the invention provides the use of the polynucleotide coding for a CAR, a CAR polypeptide, a vector, a host cell or a pharmaceutical composition as described herein in the manufacturing of a medicament for the treatment an EDA-positive cancer in a subject in need thereof. Preferably, the use of the cell of the present invention or the pharmaceutical composition of the present invention in the manufacturing of a medicament for the treatment an EDA-positive cancer in a subject in need thereof.

In some embodiments, the subject is administered a therapeutically effective amount of cells. In some embodiments, the patient is administered at least 10², 10³, 10⁴, 10⁵, 10⁶, 10⁷, 10⁸, 10⁹ or 10¹⁰ cells. The number of cells will depend upon the ultimate use for which the composition is intended as will the type of cells included therein. For example, if cells that are specific for a particular antigen are desired, then the population will contain greater than 70%, generally greater than 80%, 85% and 90-95% of such cells. For uses provided herein, the cells are generally in a volume of a liter or less, can be 500 ml or less, even 250 ml or less, or 100 ml or less. The clinically relevant number of cells can be apportioned into multiple infusions that cumulatively equal or exceed 10², 10³, 10⁴, 10⁵, 10⁶, 10⁷, 10⁸, 10⁹ or 10¹⁰ cells.

The polynucleotide coding for a CAR, the encoded CAR polypeptide, a vector, a host cell or a pharmaceutical composition as described herein, preferably a cell or pharmaceutical composition as described herein, can be administered as a single agent or in combination with another therapy or drug. In some embodiments, any of these can be administered to an EDA-positive cancer patient in combination with another anti-cancer treatment, preferably with another anti-cancer agent or radiotherapy.

The term anti-cancer agent has been defined herein above and may include but is not limited to chemotherapeutic agents, growth inhibitory agents, cytotoxic agents, anti-hormonal agents, agents used in radiation therapy, anti-angiogenesis agents, apoptotic agents, anti-tubulin agents, etc. and any combinations thereof. Said anti-cancer agent may be administered prior, concomitantly or after the agent of the present invention as described herein (e.g., the CAR cells or pharmaceutical composition of the invention). The two drugs may form part of the same composition or be provided as a separate composition for administration at the same time or at a different time. In some embodiments, said agent is an immune checkpoint inhibitor.

Immune checkpoints regulate T cell function in the immune system. T cells play a central role in cell-mediated immunity. Checkpoint proteins interact with specific ligands which send a signal into the T cell and essentially switch off or inhibit T cell function. Cancer cells take advantage of this system by driving high levels of expression of checkpoint proteins on their surface which results in control of the T cells expressing checkpoint proteins on the surface of T cells that enter the tumor microenvironment, thus suppressing the anti-cancer immune response. As such, inhibition of checkpoint proteins would result in restoration of T cell function and an immune response to the cancer cells. Examples of checkpoint proteins include, but are not limited to CTLA-4, PDL1, PDL2, PD1, B7-H3, B7-H4, BTLA, HVEM, TIM3, GAL9, LAG3, VISTA, KIR, 2B4 (belongs to the CD2 family of molecules and is expressed on all NK, γδ, and memory CD8⁺ (αβ) T cells), CD 160 (also referred to as BY55), CGEN-15049, CHK 1 and CHK2 kinases, A2aR and various B-7 family ligands. In some embodiments, the agent of the present invention as described herein (e.g., the CAR cells or composition of the invention) is used in combination with anti-PD1 or anti-PDL1 agents, such as anti-PD1 or anti-PDL-1 antibodies.

In some embodiments, the patient to be treated with the method of the present invention is in complete or near-complete remission after treatment with another therapy. In some embodiments, it may be preferable desirable to decrease the tumor burden before using the methods of the present invention. In some embodiments, the patient to be treated with the method of the present invention has previously been treated with another therapy which resulted in a partial response, complete response, stable disease, decrease in progressive disease, reduced time to tumor progression or any combination thereof.

In some embodiments, the EDA-positive cancer is colorectal cancer, liver cancer, pancreatic cancer, breast cancer, ovary cancer, prostate cancer, testis cancer, bladder cancer, glioma, melanoma, lymphoma, head and neck cancer, or cholangiocarcinoma. In preferred embodiments, the EDA-positive cancer is hepatocarcinoma, pancreatic carcinoma or teratocarcinoma (e.g., testis teratocarcinoma). In some embodiments, said EDA-positive cancer is a solid tumor. In certain embodiments, the cancer is a primary tumor. In other embodiments, the cancer is metastatic cancer. In certain embodiments, the method of treatment of the invention is for the prevention or treatment of metastasis, such as preventing metastatic recurrence. In certain embodiments, the number and/or size of metastases is reduced.

It has been described that cellular fibronectin is synthesized by many cell types, including fibroblasts, endothelial cells, myocytes or tumor cells (Spada S. et al. 2021). In some embodiments, the EDA-positive cancer is a cancer wherein EDA expression is mainly found in the tumor stroma and the endothelial vasculature, with substantially no EDA expression in the tumoral epithelial cells. For instance, EDA expression may occur mainly in the basal membrane of the tumoral endothelium as shown for cholangiocarcinoma (FIG. 1C) or teratocarcinoma (FIG. 1H or FIG. 2C). In other instances, EDA expression is found in the tumor ECM in general, such as in pancreatic cancer (FIG. 1C). In other embodiments, EDA expression may be found both in the ECM, the basal membrane of the endothelium and in some tumoral cells, such as in hepatocarcinoma (FIG. 1C).

For some cancer types, it is already known or may in the future be commonly known that these express EDA, such as previously described by Rybak J N et al., 2007; Glukhova M A et al., 1990, and as described herein, such as one or more of colorectal cancer, liver cancer, pancreatic cancer, breast cancer, ovary cancer, prostate cancer, testis cancer, bladder cancer, glioma, melanoma, lymphoma, head and neck cancer, teratocarcinoma and cholangiocarcinoma. In such embodiments, determination of whether the cancer is “EDA-positive” will generally not be necessary before implementing the methods of treatment of the invention.

For some other cancer types, it may be necessary to determine whether the tumor expresses EDA, in other words, whether it is “EDA-positive”. A person skilled in the art, will know how to determine whether a cancer is EDA-positive by well-established methods.

In some embodiments, determination of EDA expression is conducted in a tumor biopsy sample isolated from the subject, for instance obtained from a resected tumor. Tumors or portions thereof may be surgically resected from the patient or obtained by routine biopsy. Preferably, a tumor sample is obtained from the primary tumor. These types of samples are routinely used in the clinical practice and a person skilled in the art will know how to identify the most appropriate means for their obtaining and preservation. Once a sample has been obtained, it may be used fresh, it may be frozen or preserved using appropriate means (e.g., as a formalin-fixed, paraffin-embedded tissue sample). Such biological samples can be taken around the time of diagnosis, before, during or after treatment (e.g., surgical resection).

Methods for quantifying gene expression are well known in the art. Techniques to assay levels of individual biomarkers from test samples are well known to the skilled technician, and the invention is not limited by the means by which the components are assessed.

The mRNA expression level of a protein marker, such as EDA, can be determined by molecular biology methods for measuring quantities of target nucleic acid sequences well known in the art. These methods include but are not limited to end point PCR, competitive PCR, reverse transcriptase-PCR (RT-PCR), quantitative PCR (qPCR), reverse transcriptase qPCR (RT-qPCR), PCR-pyrosequencing, PCR-ELISA, DNA microarrays, gene expression panels (e.g. nanoString™), nucleic acid sequencing, such as next generation sequencing methods, in situ hybridization assays (such as dot-blot, Fluorescence In Situ Hybridization assay (FISH), RNA-ISH, automated quantitative RNA ISH (RNAscope®)), mass spectrometry, branched DNA (Nolte, Adv. Clin. Chem. 1998, 33:201-235) and to multiplex versions of said methods (see for instance, Andoh et al., Current Pharmaceutical Design, 2009; 15, 2066-2073) and the next generation of any of the techniques listed and combinations thereof, all of which are within the scope of the present invention. Such methods may also include the pre-conversion of mRNA into cDNA by the reaction with a reverse transcriptase (RT), for example the PCR or qPCR reaction is usually preceded by conversion of mRNA into cDNA and referred to as RT-PCR or RT-qPCR, respectively.

Diverse next-generation sequencing methods have been described and are well known to a person skilled in the art. These include for instance sequencing by synthesis with cyclic reversible termination approaches (e.g., Illumina, SEQLL, Qiagen), sequencing by synthesis with single-nucleotide addition approaches (e.g., Roche-454, Thermo Fisher-Ion Torrent), sequencing by ligation (e.g., Thermo Fisher SOLID and BGI-Complete Genomics), real-time long-read sequencing (e.g., Pacific Biosciences, Oxford Nanopore Technologies), synthetic long-read sequencing (e.g., Illumina, 10× Genomics, iGenomeX), see for instance Goodwin S, et al., Nat Rev Genet. 2016, 17 (6): 333-51).

In some embodiments, said molecular biology quantification methods are based on sequence specific amplification. Such an amplification-based assay comprises an amplification step which comprises contacting a sample (preferably an isolated DNA sample) with two or more amplification oligonucleotides specific for a target sequence in a target nucleic acid to produce an amplified product if the target nucleic sequence is present in the sample. Suitable amplification methods include for example, replicase-mediated amplification, ligase chain reaction (LCR), strand-displacement amplification (SDA), transcription mediated amplification (TMA) and polymerase chain reaction (PCR), which includes quantitative PCR.

One particularly preferred quantification method is quantitative PCR (qPCR), also known as real-time PCR. It relates to a type of PCR that amplifies and simultaneously quantifies a target DNA molecule. Its key feature is that the amplified DNA is detected as the reaction progresses in real time. Unless otherwise provided, the term qPCR as used herein encompasses reverse transcriptase (RT)-qPCR. Different instruments are available, such as ABI Prism 7700 SDS, GeneAmp 5700 SDS, ABI Prism 7900 HT SDS from Applied Biosystems; iCycler iQ from Bio-Rad; Smart Cycler from Cepheid; Rotor-Gene from Corbett Research; LightCycler from Roche Molecular Biochemicals and Mx4000 Multiplex from Stratagene. The qPCR process enables accurate quantification of the PCR product in real-time by measuring PCR product accumulation very early in the exponential phase of the reaction, thus reducing bias in the quantification linked to the PCR amplification efficiency occurring in end-point PCR. Real-time PCR is well known in the art and is thus not described in detail herein. Technology overview and protocols for qPCR are available for instance from the above-mentioned vendors, e.g., http://www.sigmaaldrich.com/technical-documents/protocols/biology/sybr-green-qpcr.html or http://www.sigmaaldrich.com/life-science/molecular-biology/pcr/quantitative-pcr/qpcr-technical-guide.html. For a review of qPCR methods see Wong M L y Medrano J F, Biotechniques 2005, 39 (1): 75-85. In a particular embodiment, the quantification method is a multiplex qPCR.

Expression levels may be absolute or relative. When the expression levels are normalized, normalization can be performed with respect to different measures in the sample. These procedures are well known to one skilled in the art. When gene expression is determined at the mRNA level, expression levels are typically normalized with respect to an “endogenous control”. An “endogenous control” as used herein may relate to a gene expression product whose expression levels do not change or change only in limited amounts in tumor cells with respect to non-tumorigenic cells. Housekeeping genes that can be used as endogenous control include for example ß-2-microglobulin, ubiquitin, 18-S ribosomal protein, cyclophilin, GAPDH, actin and HPRT.

In other embodiments, the determination of the expression of a protein marker, such as EDA, is carried out at protein level. Suitable methods for determining the levels of a given protein include, without limitation, those described herein below. Preferred methods for determining the protein expression are immunoassays. Various types of immunoassays are known to one skilled in the art for the quantitation of proteins of interest. These methods are based on the use of affinity reagents, which may be any antibody or ligand specifically binding to the target protein or to a fragment thereof, wherein said affinity reagent is preferably labeled. Illustrative, but non-exclusive, examples of labels that can be used include radioactive isotopes, enzymes, fluorophores, chemoluminescent reagents, enzyme cofactors or substrates, enzyme inhibitors, particles, dyes, etc.

Affinity reagents may be any antibody or ligand specifically binding to the target protein or to a fragment thereof. Affinity ligands may include proteins, peptides, peptide aptamers, affimers and other target specific protein scaffolds, like antibody-mimetics.

Specific antibodies against the protein markers used in the methods of the invention may be produced for example by immunizing a host with a protein of the present invention or a fragment thereof. Likewise, peptides specific against the protein markers used in the methods of the invention may be produced by screening synthetic peptide libraries.

Western blot or immunoblotting techniques allow comparison of relative abundance of proteins separated by an electrophoretic gel (e.g., native proteins by 3-D structure or denatured proteins by the length of the polypeptide). Immunoblotting techniques use antibodies (or other specific ligands in related techniques) to identify target proteins among a number of unrelated protein species. They involve identification of protein target via antigen-antibody (or protein-ligand) specific reactions. Proteins are typically separated by electrophoresis and transferred onto a sheet of polymeric material (generally nitrocellulose, nylon, or polyvinylidene difluoride). Dot and slot blots are simplified procedures in which protein samples are not separated by electrophoresis but immobilized directly onto a membrane.

Traditionally, quantification of proteins in solution has been carried out by immunoassays on a solid support. Said immunoassay may be for example an enzyme-linked immunosorbent assay (ELISA), a fluorescent immunosorbent assay (FIA), a chemiluminescence immunoassay (CIA), or a radioimmunoassay (RIA), an enzyme multiplied immunoassay, a solid phase radioimmunoassay (SPROA), a fluorescence polarization (FP) assay, a fluorescence resonance energy transfer (FRET) assay, a time-resolved fluorescence resonance energy transfer (TR-FRET) assay, a surface plasmon resonance (SPR) assay. Multiplex and any next generation versions of any of the above, such as bead-based flow-cytometry immunoassays (e.g., based on the Luminex xMAP technology) are specifically encompassed. In a particular embodiment, said immunoassay is an ELISA assay or any multiplex version thereof.

Other methods that can be used for quantification of proteins in solution are techniques based on mass spectrometry (MS) such as liquid chromatography coupled to mass spectrometry (LC/MS), described for example in US2010/0173786, or tandem LC-MS/MS (WO2012/155019, US2011/0039287, M. Rauh, J Chromatogr B Analyt Technol Biomed Life Sci 2012 Feb. 1, 883-884. 59-67) and multiplex versions of the above techniques, as well as the next generation of such techniques and combinations thereof.

For determining protein expression and location, immunohistochemical and in-situ hybridization analysis are usually preferred. Multiplex versions thereof are also encompassed.

Immunohistochemistry (IHC) analysis is typically conducted using thin sections of the biological sample immobilized on coated slides. These sections, when derived from paraffin-embedded tissue samples, are deparaffinised and preferably treated so as to retrieve the antigen. The detection can be carried out in individual samples or in tissue microarrays.

Immunohistochemical detection of a particular protein marker, such as EDA, may be conducted in a tumor sample, such as a tumor biopsy, using the corresponding healthy tissue as control. Quantification of EDA expression in the indicated tumor samples and healthy tissues can be measured by using a software for image analysis, such as ImageJ software. For instance, a number of fields (e.g., 10 fields) can randomly be selected in each tumor section and then the densitometry of each of them is to be quantified using the ImageJ program. For instance, the value of each field can be considered positive when it exceeded the mean value+2SD of the densitometry of an immunostaining obtained with a negative control antibody.

In some embodiments, the CAR nucleic acid, the encoded CAR polypeptide, a vector, a host cell or a pharmaceutical composition as described herein, preferably a cell or pharmaceutical composition as described herein, is characterized by being well tolerated and/or by the absence of signs of toxicity, specially “on-target/off-tumor” toxicity when administered in a therapeutically effective amount thereof in a method of treating an EDA-positive cancer as described herein. In some embodiments, the method of treatment is characterized by absence of CAR-T cell activation in healthy tissues.

In some embodiments, the method of treating cancer described herein is characterized by one or more of the following effects:
reduces or inhibits angiogenesis;
reduces the amount of fibrosis in the tumor, for instance as shown by a reduction of the expression of fibrotic markers, such as α-SMA;
increases immune system cells penetration into the tumor;
reduces or inhibits the tumor growth;
reduces the gene signatures associated with epithelial-mesenchymal transition, collagen synthesis or extracellular matrix organization; and/or
reduces gene signatures associated with inflammatory processes, such as IL-6-STAT5 and KRAS pathways.

In some embodiments, the method of treating cancer described herein results in an inhibition of tumor growth, a reduction of the number and/or size of tumors, a partial response, complete response, stable disease, decrease in progressive disease, reduced time to tumor progression or any combination thereof.

### EDA-CAR Cells in the Treatment or Prevention of Diseases Mediated by an Excessive or Inadequate Response of the Immune System

Cell therapy with Tregs in the treatment of diseases mediated by an excessive or inadequate response of the immune system, such as autoimmune processes (Bluestone J. A., et al., 2015, Expert Opin. Ther. Targets; 19:1091-103), graft-versus-host disease in bone marrow transplant patients (Brunstein C. G., et al., 2016, Blood; 127:1044-51) or transplant rejection (Safinia N., et al., 2015, Front Immunol; 6:438) has attracted great attention in the recent years.

The basic role of Tregs in transplants has been confirmed by various studies in animal models of skin and heart transplants, demonstrating that the Tregs present in the receptacle at the time of the transplant are critical to the induction and maintenance of tolerance to the graft (Wood K. J., Sakaguchi S., 2003, Nat. Rev. Immunol.; 3:199-210). These Treg cells will impede the activation and expansion of effector T cells, which are responsible for cellular rejection. Additionally, Tregs can also induce the death of B cells, preventing humoral rejection, as already demonstrated in a cardiac xenotransplantation model (Ma Y., et al., 2008, Xenotransplantation; 15:56-63).

CAR-Tregs have been disclosed as a promising tool for inducing immunological tolerance and in the treatment of autoimmune diseases. When delivered into the patient, the CAR-Tregs would migrate to the target sites and bind to target antigens, activating and helping to maintain immune homeostasis and prevent autoimmune disease. CAR-Tregs have been described to exert a more potent and specific immunosuppression than do polyclonal Tregs (Zhang Q, et al. Front Immunol. 2018 Oct. 12; 9:2359).

In a further aspect, the invention relates to CAR Tregs of the invention for use in the treatment or prevention of diseases mediated by an excessive or inadequate response of the immune system, such as transplant rejection, especially in cardiac transplant rejection. It further pertains to related methods of treatment.

During cardiac tissue remodelling occurring in different cardiovascular disorders it has been observed the expression of fetal variants of Fibronectin (Fn) and Tenascin-C(Tn-C) generated by alternative splicing. It has been described that these molecular variants are virtually absent in the healthy adult human organ while these are highly expressed during cardiovascular remodelling. In particular ED-A domain containing Fibronectin (EDA-FN) and A1 domain containing Tenascin-C(A1 Tn-C) have been suggested to be extensively re-expressed during myocardial tissue reorganisation in coronary artery disease or aortic valve stenosis (Franz M, et al., J Mol Histol. 2014 October; 45 (5): 519-32). It has further been described that the expression of EDA in the recipient of cardiac allografts promotes cardiac allograft fibrosis which is associated with chronic rejection (Booth A J, et al., J Pathol. 2012 March; 226 (4): 609-18).

In some embodiments, the subject is administered a therapeutically effective amount of cells. In some embodiments, the patient is administered at least 10², 10³, 10⁴, 10⁵, 10⁶, 10⁷, 10⁸, 10⁹ or 10¹⁰ cells. The number of cells will depend upon the ultimate use for which the composition is intended as will the type of cells included therein. For example, if cells that are specific for a particular antigen are desired, then the population will contain greater than 70%, generally greater than 80%, 85% and 90-95% of such cells. For uses provided herein, the cells are generally in a volume of a liter or less, can be 500 ml or less, even 250 ml or less, or 100 ml or less. The clinically relevant number of cells can be apportioned into multiple infusions that cumulatively equal or exceed 10², 10³, 10⁴, 10⁵, 10⁶, 10⁷, 10⁸, 10⁹ or 10¹⁰ cells.

### Kit of the Invention

The invention further provides a kit which comprises a dosage form of a CAR nucleic acid, the encoded CAR polypeptide, a vector, a host cell or a pharmaceutical composition as described herein, preferably a cell or pharmaceutical composition as described herein; optionally with a dosage form of another drug; and instructions for the use thereof. The containers in which the compound or pharmaceutical composition is supplied can be any conventional container that is capable of holding the supplied dosage forms.

It is contemplated that any embodiment discussed in this specification can be implemented with respect to any polynucleotide, polypeptide, vector, cell, medical use, pharmaceutical composition, kit, method of treatment, method of manufacturing a medicament and combination therapies of the invention, and vice versa. It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications are herein incorporated by reference to the same extent as if each individual publication or patent application was specifically and individually indicated to be incorporated by reference.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” or “around” may vary from the stated value by ±1, 2, 3, 4, 5, 6, 7, 10, 12 or 15%.

### Examples

### I. Materials & Methods

The assays disclosed below were carried out using the following materials and methods:

### Data Acquisition

Data from TCGA samples for 20 different tumor types and adjacent normal samples were explored for changes of alternative splicing events (exon 33 skip (EDA)) using SpliceSeq tool (version 2.1) (Ryan M C et al., 2012) (http://projects.insilico.us.com/TCGASpliceSeg/). The percent spliced in (PSI) value was calculated to quantify alternative splicing events ranging from 0 to 1 in TCGA SpliceSeq (Ryan M et al., 2016). From a subgroup of 15 different tumor types, only pair samples including also the EDA expression in the normal tissue surrounding the tumor were considered. The percent-splice-in (PSI) value was calculated to quantify alternative splicing events ranging from 0 to 1 in TCGA SpliceSeq (Ryan M et al., 2016) The ratio of normalized read counts indicating inclusion of EDA element over the total normalized reads for that event (both inclusion and exclusion reads).

EDA alternative splicing events were also evaluated in a cohort of 17 hepatocellular carcinoma (HCC) patients with available RNASec data from our institution. The ratio between the expression levels (read counts) of exon 33 (EDA) and exon 34 (included in all fibronectin isoforms) in both tumor and normal hepatic tissue was used as an estimation of EDA expression in HCC.

### Mice

Female C57BL/6 J mice were purchased from Harlan Laboratories. 129Sv mice were obtained from Janvier Laboratory (Le Genest Saint Isle, France). NSG mice were bred in our animal facilities at the Centro de Investigación Medica Aplicada. All animal handling and tumor experiments were approved and conducted under the institutional guidelines of our institutional ethics committee (Ref: 019-19) and following the European Directive 2010/63/EU.

### Cell Lines

The murine hepatocellular carcinoma PM-299L (provided by Dr. Lujambio, NY), the murine melanoma B16F10 (ATCC CRL-6475), the murine testicular teratoma F9 (ATCC CRL-1720), the human hepatocarcinoma PLC and HuH7 and the human cholangiocarcinoma HUCCT and TFK1 cell lines (provided by Dr. Ávila, Pamplona, Spain) were cultured in complete medium (RPMI 1640 or DMEM containing 10% FCS, antibiotics, 2 mM glutamine and 50 μM 2-ME). Jurkat cells transfected with a triple parameter reporter (TPR) system (Rosskopf S et al. 2018), provided by Dr Hudecek (Würzburg, Germany) and cultured in complete DMEM medium, allow the measurement by flow cytometry of the main CAR-mediated activation signaling pathways (NFAT, NFkB and AP1 pathways) after antigen recognition (Jutz S et al., 2016). The PM299L-Thy1.1 and PM299L-EDA cell lines were generated by retroviral transduction of PM299L-WT cells with a retrovirus expressing EDA fused to the transmembrane domain of PDGFR and the membrane cell marker Thy1.1 (RV-EDA-PDGFR-IRES-Thy1.1 plasmid). A cell line expressing only the Thy1.1 cell marker was also generated. Three different PM299L-EDA cell clones were isolated to express low (clone C3), intermediate (clone C2) and high (clone C7) levels of EDA on the cell membrane. The Platinum Ecotropic (Plat-E, ATCC) and HEK293T cell lines were cultured in DMEM supplemented with 10% FCS and the selection antibiotics puromycin (100 ug/ml) and blasticidin (10 ug/ml). All cell lines were cultured at 37° C. in a humidified atmosphere with 6.5% CO2.

Expression and Purification of Anti-EDA scFv (F8)

A DNA sequence coding for the scFv recombinant antibody fragment F8 (Rybak J N et al., 2007) was cloned in pET20b plasmid (Novagen), which enables expression of fusion proteins carrying six histidine residues at the carboxyl terminus. The resulting plasmid was transfected into BL21(DE3) cells for the expression of the recombinant scFv which was purified by affinity chromatography using protein A Sepharose (Amersham Biosciences) according to the manufacturer's instructions. Purified antibody fragments were analyzed by Coomassie and Western blot using anti-His antibodies. Recognition of EDA protein by the purified scFv F8 fragment was tested by ELISA using plates coated with recombinant EDA protein produced as described previously (Lasarte J J et al., 2007). The equilibrium dissociation constant (K_{D}) of scFv F8 to soluble human and mouse EDAs was calculated by Bio-Layer Interferometry (BLI) using an Octet N1 (Sartorius) (FIG. 13). The scFv F8 was coated to the AR2G Biosensors (Fortébio) following manufacturer's instructions.

### Plasmids and Viral Transduction

### Murine Chimeric EDA CAR

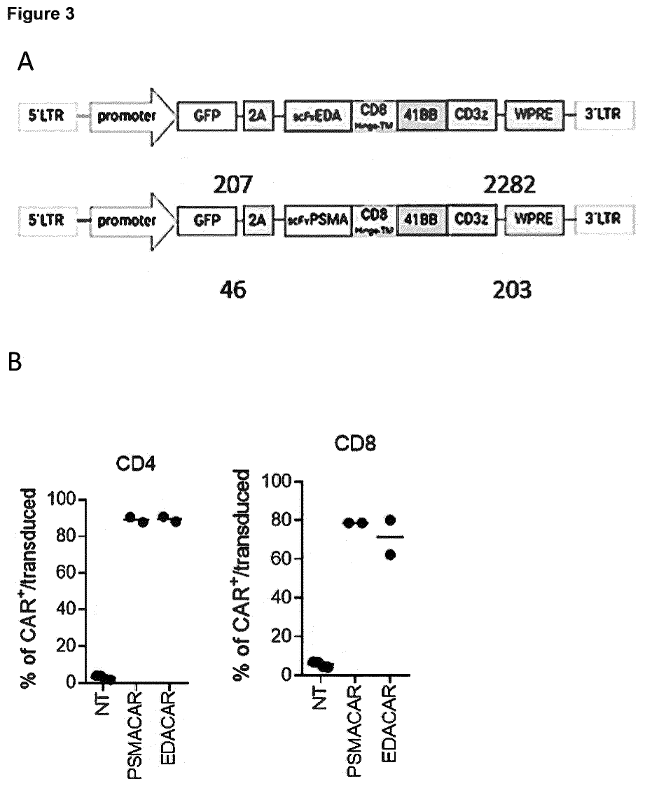

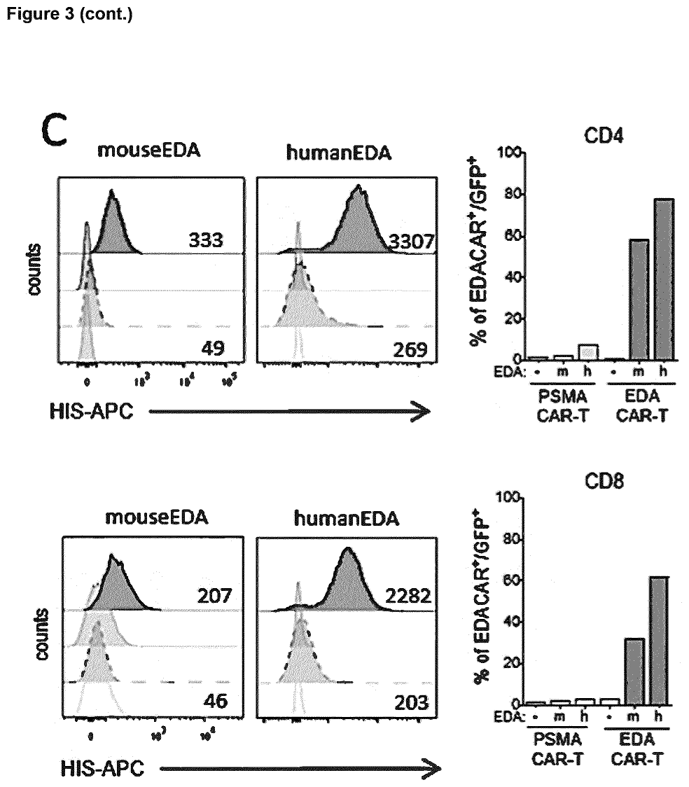

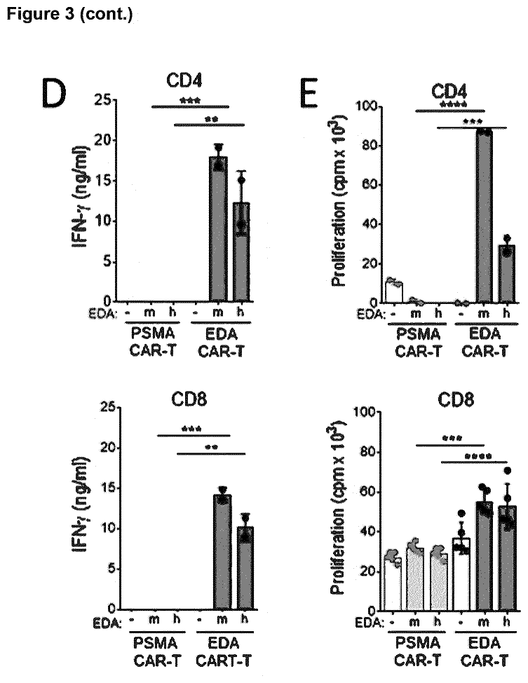

The first chimeric EDA CAR used to generate the murine anti-EDA CAR T cells is composed of the anti-EDA F8 scFv (SEQ ID NO: 9) and a murine 4-1BB-CD3ζ expression cassette linked through a F2A self-cleaving peptide sequence to eGFP (FIG. 2A, SEQ ID NO: 22). The PSMA CAR, used as an irrelevant CAR, included the anti-human PSMA scFv obtained from mouse hybridoma J591 (PTA-3709 ATCC), that was cloned in the same expression cassette. Two other chimeric EDA CARs were generated containing (i) the anti-EDA C27 ScFv (SEQ ID NO: 26) or (ii) the anti EDA C33 ScFv (SEQ ID NO: 28), respectively, instead of the scFv F8, which were also cloned in the same expression cassette. All these constructs were cloned in the pRubiG cassete (modifying the backbone of pRubiC-T2A-cre (Addgene #66692 plasmid) (see FIG. 3A) for retrovirus production. These plasmids were synthesized by Genscript.

### Human Chimeric EDA CAR

The EDA CAR used to produce human CAR-T cells (SEQ ID NO: 20) also contained the anti-EDA F8 scFv and the human endodomains 4-1BB-CD3ζ. This cassette was cloned in a third-generation self-inactivating lentiviral vector (LV) and regulated under an EF1α promoter. To facilitate the quantification of the transduction, LV EDA CAR also included the reporter gene blue fluorescent protein (BFP). These plasmids were synthesized by Genscript.

For retrovirus productions, Plat-E cells (Invitrogen, Toulouse, France) were transfected with 5 μg of retroviral plasmid DNA along with 2.5 μg pCL-Eco plasmid (Addgene plasmid #12371; Naviaux R K et al. 1996) DNA using lipofectamine 2000 (Invitrogen) for 6 h in antibiotic-free medium. Retroviral supernatants were collected at 48 and 72 h. For the retroviral transduction of lymphocytes, the supernatants were spun at 2000×g for 90 min at 32° C. into 12-well plates with mouse preactivated CD4 or CD8 with coated beads CD3/CD28 (BD). Lymphocytes were cultured with 50 IU of recombinant human IL-2.

For lentivirus production, replication-defective lentiviral vectors comprising the EDA-CAR, were produced in HEK293T cells using a third generation lentiviral packaging system [pMD2.G: VSV-G envelope expressing plasmid (Addgene plasmid #12259); pRSV-Rev: Lentiviral packaging plasmid—contains Rev (Addgene plasmid #12253) and pMDLg/pRRE: Lentiviral packaging plasmid—contains Gag and Pol (Addgene plasmid #12251); Dull T et al. 1998]. Briefly, 6×10⁶ cells were plated in p100 culture dishes and 24 h later were cotransfected with 6.9 μg of the CAR expressing plasmid, 3.41 μg pMDLg/pRRE (Gag/Pol), 1.7 μg pRSVRev and 2 μg pMD2.G (VSVG envelope) packaging plasmid using lipofectamine 2000. Forty hours after transfection, supernatants were collected, filtered and concentrated using the Lenti-X Concentrator (Takara) following the manufacturer's specifications. T cells were infected with the LV at MOI 3 and 10 μl/ml of LentiBoost (Sirion Biotech) to enhance viral infection. Transduction efficiency was evaluated by FACS measuring reporter gene expression.

### Antibodies and Flow Cytometry

Samples were tested with a FACS Canto II flow cytometer (Becton Dickinson) and data were analyzed by FlowJo software (TreeStar). EDA and control PSMA CARs were detected by the expression of GFP and/or by using Biotin-SP-conjugated anti-human or anti-mouse immunoglobulin G antibodies (109-066-097 and 115-066-072 respectively, Jackson Immunoresearch), followed by incubation with fluorochrome-conjugated Streptavidin (Biolegend).

The transduced PM299L-WT cells (PM299L-EDA or PM299L-Thy1.1) were sorted by using an anti-Thy1.1 antibody (OX-7, Biolegend). EDA expression in tumor cell lines was tested though flow cytometry with anti-EDA scFv (F8) antibody followed by a fluorochrome-conjugated anti-6x-His Tag antibody.

T-cell phenotype was assessed by using the following fluorochrome-conjugated antibodies (Biolegend) used at 0.25-1 μg/ml: CD8a (53-6.7), CD4, (RM4-5) CD45.1 (A20), CD45.2 (104), CD44 (IM7), CD62L (MEL-14), CD137 (17B5), PD-1 (29F.1A12), TIGIT (1G9) and LAG3 (C9B7W). For staining, cells were incubated with the Zombie NIR™ Fixable Viability kit (Biolegend) for 15 min at room temperature and then washed once with washing buffer. Cells were fixed and permeabilized with the Foxp3/Transcription Factor Staining kit buffers (eBioscence) and then stained intracellularly (15 min, RT) with fluorochrome-conjugated mAbs against mouse, Ki67 (16A8), TNF-α (MP6-XT22) and IFN-γ (XMG1.2). Perfect-Count beads (Cytognos) were added for the flow cytometry quantification of absolute cell numbers.

### Immunohistochemistry

For immunohistochemichal detection of EDA, α-SMA, CD31 or collagen IV in human or mouse paraffin embedded tumor tissue sections, antigen retrieval was performed by heating the samples in a microwave oven using citrate buffer (10 mM, pH 9 for EDA, and pH 6 for CD31 and collagen IV). After blocking non-specific binding sites, the primary antibody scFv F8 was added and incubated at 5 ug/ml overnight at 4° C. After washing with TBS solution, slides were incubated with the anti-6x-His Tag antibody (RM146, Abcam) at a 1:200 dilution for 45 minutes at room temperature. After TBS washes, samples were incubated with Dako EnVision+ System-HRP Labelled Polymer Anti-Rabbit during 30 minutes at room temperature. For quantification, slides were scanned with the Aperio CS2 scanner (Leica, Barcelona, Spain) and images were visualized with the Aperio Image Scope (v12.1.05029). Slides were scanned with the Aperio CS2 scanner (Leica, Barcelona, Spain) and images were visualized with the Aperio Image Scope (v12.1.05029). For quantification, ten non-overlapping fields were randomly selected from each tissue preparation, and optical density (DAB chromogen signal corresponding to EDA protein expression) was calculated in all tissue slides using ImageJ software using the Colour Deconvolution plugin. In human tumor samples, the obtained signal was compared with that corresponding to each healthy tissue stained with the scfvF8. The rest of the samples were compared with the negative control consisting of the same slide only stained with the secondary antibody.

### Multiplexed Immunohistochemistry

Paraffin-embedded tissue sections were used for Multiplexed immunohistochemistry using anti EDA scFv F8 antibody and anti-human CD31 (77699S, Cell Signaling) and Collagen IV (ab6586, Abcam). Samples were stained using an Opal 4-color anti Rabbit Kit (NEL840001KT a validated kit from Akoya Biosciences (Marlborough, Massachusetts, USA), according to the manufacturer's recommendation. Samples were counterstained using DAPI and digitalized using a Phenolmager HT scanner (Akoya Biosciences).

### Characterization of CAR-T Cells

### CAR Expression on Surface and Binding to EDA Protein

The CAR expression was measured by flow cytometry, 1*10⁵ EDA or PMSA CAR-T cell were incubated with biotinylated anti-human or anti mouse IgG, respectively, at 1 μg/ml of (Jackson ImmunoReseach) for 40 min at 37° C. After washing, cells were stained with streptavidin-APC conjugated for 15 min at room temperature.

To evaluate the capacity of the EDA-CAR T cells to bind EDA, cells were incubated with murine and human recombinant EDA proteins at 5 ug/ml. After 20 min of incubation with the protein at 37° C., cells were washed and labelled with the anti-HIS tag AF647 antibody during 15 min at room temperature and analyzed by flow cytometry.

### CAR-T Cell Response to EDA

5×10⁵ CAR-T cells were plated in 96-well plates pre-coated with recombinant EDA or recombinant ovalbumin OVA (Endograde) as an irrelevant protein for 48 h. Also CAR-T cells were plated for 24 h in the presence of irradiated PM299L-EDA and PM299L-Thy1.1 cells. The number of IFN-γ producing cells was measured by ELISPOT as described by Lozano et al. (Lozano T et al., 2019). CAR-T cell proliferation and IFN-γ production were measured by 3H-thymidine incorporation (0.5 μCi per well) and ELISA, respectively, as described by Casares et al. (Casares N et al., 2010). In some cases, CAR-T cell proliferation was measured by Cell trace violet (CTV) dilution assay; Lymphocytes were incubated with the dye (5 μM) for 15 minutes at 37° C. and washed with RPMI 10% FBS. 8×10⁵ labelled lymphocytes were plated and co-incubated with PM299L-EDA^{high} and PM299L-Thy1.1 tumor cells at a ratio of 1:1 for 72 hours at 37° C. Subsequently, the proliferating population (measured by CTV dilution) was analyzed within GFP+ lymphocytes. The proliferation index was calculated as the ratio between proliferating cells in the presence of tumor cells expressing EDA (PM299L-EDA^{high}) and the number of proliferating cells in the presence of tumor cells not expressing EDA (PM299L. Thy1.1) at the end of the co-culture.

### Cytotoxicity Assays

Real-time cytotoxicity assay (xCELLigence) was carried out to analyze the cytotoxicity of the CD8⁺ CAR-T cells as previously described (Lozano T et al., 2019) and using two different Effector: Tumor cell ratios (1:1 and 0.2:1). All experiments were performed in duplicate.

CAR-T cell cytotoxicity was also measured by flow cytometry. 6×10⁵ CAR-T cells were cocultured with PM299L-EDA or PM299L-Thy1.1 cells for 24 h at two different Effector: Tumor ratios (1:1 and 0.2:1). Then, cells were washed and incubated with a fluorochrome-conjugated antibody against CD8. Perfect-Count beads (Cytognos) were added for the flow cytometric quantification of absolute cell numbers.

### In Vivo Assessment of Anti EDA CAR-T Cell Antitumor Activity

Winn assay. C57BL/6 mice (8-10 weeks of age) were sublethally irradiated (total body irradiation) with 4 Gy. Then, mice were injected with 2×10⁶ PM299L-EDA C2 cells by the subcutaneous (s.c) route and received 2×10⁶ CD4+ and 6×10⁶ CD8+ CAR-T cells by the intravenous (i.v) route.

PM299L-EDA established tumor. C57BL/6 female mice (8-10 weeks of age) were injected with 2×10⁶ PM299L-EDA C7 cells by the s.c route. Seven days later, mice were sublethally irradiated with 4 Gy and received 9×10⁶ CAR-T cells.

F9 established tumor. 129Sv mice (8-10 weeks of age) were injected with 3×10⁶ F9 cells by the subcutaneous (s.c.) route. Seven days later, mice were sublethally irradiated and received 1×10⁷ CAR-T cells (CD4 and CD8 ratio 5:1).

All mice received also 20,000 U human IL2/day during 4 days after T cell infusion.

Xenograft mouse model. Eight to 10-week-old male/female NSG mice obtained from the CIMA NSG colony were injected s.c. with 3×10⁶ PLC tumor cells embedded in Matrigel (Corning; 1:1 diluted in PBS). On day 8 after tumor injection, when tumors were 5-7 mm in diameter, mice received a single i.v. dose of 5×10⁶ untransduced T cells, EDA CAR-T cells, or were left untreated. In all these models, tumor area was monitored with a caliper every 2-3 d after T cell infusion. Mice were sacrificed when tumor diameter reached a value >2 cm. There were no exclusion of animals in the analyses.

### Angiosense Imaging

Mice bearing F9 tumors were injected with a single i.v. dose of Angiosense 750 (2 nmol/100 μL) as recommended by the manufacturer (NEV10011EX, PerkinElmer;). After 24 hours of Angiosense 750 adminsitration, fluorescence accumulation into the tumor was visualized using the PhotonImager Optima (Biospace, Paris, France). Relative fluorescent units were calculated by measuring tumor fluorescence divided by tumor volume.

### RNAseq Analysis

Total RNA from tumors was isolated using the MagMAX mirVana total RNA isolation Kit (Applied Biosystems). Following mechanical homogenization with an Ultra-turrax (T10 basis Ultra-Turrax, IKA), RNA was extracted according to the manufacturer's instructions and stored at −80C until processed. RNA concentration was quantified using a Qubit 3.0 Fluorometer and its quality was examined in Agilent's 4200 TapeStation System. Roughly 150 ng of quality total RNA were used for the transcriptome interrogation using the Illumina Stranded Total RNA Prep Ligation with Ribo-Zero Plus kit according to the manufacturer's instructions (Illumina). Briefly, cytoplasmic and mitochondrial rRNAs as well as beta globin transcripts were depleted from the samples. The remaining RNA was fragmented and reverse-transcribed. A second strand cDNA synthesis step removed the RNA template while incorporating dUTP in place of dTTP in order to preserve strand specificity. Next, double-stranded cDNA was A-tailed, then ligated to Illumina anchors bearing T-overhangs. PCR-amplification of the library allowed the barcoding of the samples with 10 bp dual indexes and the completion of Illumina sequences for cluster generation. Libraries were quantified with Qubit dsDNA HS Assay Kit and their profile was examined using Agilent's HS D1000 ScreenTape Assay. Sequencing was carried out in an Illumina NextSeq2000 using paired-end, dual-index sequencing (Rd1: 59 cycles; i7: 10 cycles; i5: 10 cycles Rd2: 59 cycles) at a depth of 50 million reads per sample.

RNA-seq reads are trimmed using Trim Galore v0.4.4 using default parameters to remove the Nextera adapter sequence. Mapping is performed using STAR (2.6) against the mouse NCBIM37 genome, guided by gene models from Ensembl annotation release 68. Quantification and generation of gene expression matrices were performed with the function featureCounts, implemented in the R package Rsubread. Aligned fragments are imported into RStudio and before statistical analysis, the function filterbyExpr, implemented in the R package edgeR, was used to determine genes with enough counts for further analyses. Differential gene expression analysis is performed using the DESeq2 algorithm within R and RStudio. Gene set enrichment analysis was carried out using GSEA software (https://www.gsea-msigdb.org/).

### Toxicity Studies

C57BL/6 mice bearing PM299L-EDA C7 tumors mice were sublethally irradiated and received 1×10⁷ CAR-T cells (5×10⁶ CD8 and 5×10⁶ CD4). Seven days after CAR-T cell infusion, mice were sacrificed and blood and tissues were collected. Serum biochemical parameters were measured by a Roche Cobas 6000 analyzer (Roche Diagnostics, Mannheim, Germany). Liver, spleen, lung, kidney and heart were also resected from the mice and stained with hematoxylin and eosin (H&E) for toxicity evaluation.

Naïve C57BL/6 J mice were sublethally irradiated and received 1×10⁷ CAR-T cells (5×10⁶ CD8 and 5×10⁶ CD4). Blood samples were obtained at day 17 and 30 after CAR-T cell infusion and mice were sacrificed and tissues were collected at day 30. Serum biochemical parameters were measured by a Roche Cobas 6000 analyzer (Roche Diagnostics, Mannheim, Germany). Liver, spleen, lung, kidney and heart were also resected from the mice and stained with hematoxylin and eosin (H&E) for toxicity evaluation.

### Ex Vivo Analysis of Tumor-Infiltrating T Cells

PM-299L-EDA or F9 tumors were harvested between days 4-9 after T cell injection. Excised tumors were digested with 400 U/mL collagenase-D and 50 μg/mL DNase-I (Roche) for 20 min at 37° C. For functional analyses, cells were stimulated with PMA (50 ng/ml)/lonomycin (1 μg/ml) and GolgiStop/GolgiPlug (BD Biosciences). After 5 h, cells were incubated with Zombie NIR Fixable dye (Biolegend). Subsequently, they were stained with fluorochrome-conjugated mAbs against CD45.2 (104), CD8 (XMG1.4), PD-1 (29F.1A12), LAG3 (C9B7W), and TIGIT (1G9) in the presence of purified anti-CD16/32 mAb. For intracellular staining, cells were fixed and permeabilized with the BD Fixation/Perm buffer (BD Biosciences) and stained with anti-IFN-γ (XMG1.2) and with anti-KI67 (16A8) mAbs. Samples were acquired on a FACSCanto-II cytometer (BD Biosciences). Data were analyzed using FlowJo software (TreeStar).

Flow Cytometry Analysis of the Activation of NFAT, AP1 and NF-kB in the Triple Parameter Reporter Jurkat TPR Cell Line Expressing the EDA-CAR in Response to Stimulation with Wild Type Jurkat Cells Previously Incubated with EDA Protein

Wild type Jurkat cells (Clone E6-1, ATCC) were incubated with 25 μg/ml of EDA protein (produced as described in Lasarte J J et al, 2007) for 30 min at 37° C., and used as EDA expressing cells to evaluate the capacity to be recognized by EDA CAR-T cells. We first measured the EDA binding to wild type Jurkat cells by flow cytometry by labeling EDA protein coated to the cells using anti-His-APC antibodies (anti-HIS tag AF647 (Thermo Fisher).

Then, we measured the capacity of EDA coated cells to be recognized by EDA CAR expressing Jurkat cells. Thus, TPR Jurkat cells were transduced with the CAR α-EDA, and stimulated in triplicate in the presence of wild type jurkat cells previously incubated with the EDA protein. After 24 h of co-culture, cells were analyzed by flow cytometry to evaluate the activation of the main CAR-mediated activation signaling pathways (NFAT, NFkB and AP1 pathways) using the CytoFLEX LX flow cytometer (Beckman Coulter).

### Statistical Analysis

Normality was assessed with Shapiro-Wilk W test. Statistical analyses were performed using parametric Student t tests, two-tailed paired t-tests, and one-way ANOVA with the Bonferroni multiple comparison test, as indicated. The Mann-Whitney U and Kruskal-Wallis tests were used for non-parametric analyses. For all tests a p value <0.05 was considered statistically significant. Descriptive data for continuous variables were reported as means+−SD. GraphPad Prism version 7 (GraphPad Software) was used for statistical analysis.

### EXAMPLE 1. EDA is Expressed in Human Tumor Samples.

We analyzed the EDA fibronectin (FN1) mRNA splicing pattern in a panel of 20 different tumors using The Cancer Genome Atlas (TCGA) RNASeq data and the TCGA SpliceSeq resource (http://projects.insilico.us.com/TCGASpliceSeq) (FIG. 1A.1). This analysis highlighted the upregulation of EDA in breast cancer (BRCA), cholangiocarcinoma (CHOL), liver cancer (LIHC), glioblastoma (GLB) or head and neck squamous cell carcinoma (HNSC) with respect to the EDA expression in the corresponding adjacent normal tissues. The paired analysis in FIG. 1A.2 highlighted the upregulation of EDA in human cholangiocarcinomas (CHOL), breast cancer (BRCA), head and neck squamous cell carcinoma (HNSC) and in liver cancer (LIHC), with respect to the EDA expression in the corresponding adjacent normal tissues. We also found significant differences in Colon adenocarcinoma (COAD), Kidney Renal Clear Cell Carcinoma (KIRC), Lung Adenocarcinoma (LUAD), Lung Squamous Cell Carcinoma (LUSC), Prostate Adenocarcinoma (PRAD) or Uterine Corpus Endometrial Carcinoma (UCEC). However, in these cases, the differences in mean between the tumor and the normal tissue were lower than 10%, as opposed to the strong differences (>25%) found for CHOL, BRCA, HNSC or LIHC.

We confirmed the results obtained from the TCGA data base in a cohort of 17 patients with hepatocarcinoma (HCC) with available RNASec data from our institution. HCC tumor samples had a very significant increase in EDA expression in tumor tissues compared to the corresponding adjacent non-tumoral tissue (FIGS. 1B.1 and 1B.2).

We then measured the EDA expression in different tumors at the protein level using the human antibody fragment scFv (F8) specific for the EDA domain (Rybak J N et al., 2007). These analyses confirmed and expanded the results previously reported by Rybak et al. As compared to that observed in the corresponding healthy tissue, we found strong EDA expression in human biopsies of cholangiocarcinoma, hepatocarcinoma, colon, ovarian and pancreatic cancers, whereas these differences were mild in other gastrointestinal cancers (duodenal, rectal and stomach cancers) (FIG. 1C). EDA expression compared to the corresponding negative controls was quantified using ImageJ software (FIG. 1D).

We also measured the EDA expression in tumor tissues induced in NSG mice after subcutaneous injection of human hepatocarcinoma (PLC and HuH7) and cholangiocarcinoma (HuCCT and TFK1) cell lines (FIG. 1E, 1F). In agreement with the results found in human tumor biopsies, prominent staining of tumor stroma and the neovasculature within the tumor was observed in both types of tumors. Indeed, CD31 co-localized with EDA in immunofluorescence staining in PLC tumors. EDA/CD31 co-localization experiments in tumor samples from these NSG mice challenged with PLC, HUH7, HUCCT and TFK1 human tumor cell lines, showed in all cases a broad EDA staining of tumor stroma and also in the basement membrane of the tumor neovasculature, close to CD31 staining. These images suggest that EDA is expressed in the extracellular matrix surrounding and supporting the endothelial cells but not in the endothelial cells themselves (FIG. 1G).

Since type IV collagen is the main component of the basement membrane and it plays a role in endothelial cell proliferation (Madri J A, 1997), we carried out also collagen IV-EDA co-localization experiments in PLC tumors. As control, we included tissue sections from normal murine kidney. It was found an important but not exclusive co-localization of EDA and type IV collagen in F9 and PLC tumors whereas no EDA expression was detected in the normal kidney (FIG. 1H).

It has been described that cellular fibronectin is synthesized by many cell types, including fibroblasts, endothelial cells, myocytes or tumor cells (Spada S. et al. 2021). To find out the origin of the EDA protein detected in human tumors induced in NSG mice, we used two different anti-EDA antibodies: the αEDA IST-9 antibody (Carnemolla et al., 1987) that only detects the human protein and the scFv F8 that detects the protein from both origins. It was observed that in the case of the hepatocarcinoma tumors studied (PLC and HUH7), the origin of the EDA deposited in the extracellular matrix and around the endothelium was from human origin and thus must be produced by the human tumor cell itself. However, in the case of the TFK1 cholangiocarcinoma tumor, unlike the HuCCT, the origin is from mouse cells, possibly macrophages, fibroblasts, or endothelial cells. We included the staining of a human hepatocarcinoma and a human cholangiocarcinoma to check the staining pattern of both antibodies (FIG. 1I).

### EXAMPLE 2. EDA Expression in Murine Tumor Cell Lines and Tumor Tissues.

Since F8 scFv engages human and mouse EDA protein (96.6% homology), we also measured by flow cytometry the EDA expression in different tumor murine cell lines (B16-OVA melanoma, LLC-OVA lung carcinoma and F9 teratocarcinoma) cultured in vitro. We found a marginal staining in the LLC-OVA cell line, which is upregulated when cells are cultured for 16 h in the presence of TGFβ, a well described inducer of EDA splicing (Balza E et al., 1988; Ventura E et al., 2018) (FIG. 2A). EDA expression was then analyzed in tumor tissues induced in mice after subcutaneous injection of the indicated cell lines. We found some positive staining for EDA in F9 tumors. However, no expression was detected in LLC-OVA or in B16-OVA induced tumors. As previously described for F9 tumors (Rybak J N et al., 2007), EDA staining was mainly located in endothelial cells (FIG. 2B).

### EXAMPLE 3. EDA CAR-T Cells Recognize EDA and Kill EDA Expressing Tumor Cells.

We prepared a retroviral construct encoding a cassette for the expression of EDA-specific CAR cloned in the pRubiG plasmid (Addgene Plasmid #66696) in frame with the eGFP-P2A gene to express eGFP and the CAR simultaneously. As a control we also prepared a pRubiG plasmid expressing an anti-PSMA CAR (FIG. 3A). EDA and PSMA CAR-T cells were generated by retroviral transduction of CD4⁺ or CD8⁺ T cells with the corresponding retrovirus. Five days after infection, T cells were analyzed by flow cytometry. Both CD4⁺ and CD8⁺ PSMA CAR-T and EDA CAR-T transduced cells express their respective CAR construct with an efficiency of transduction in the 80% range in both cases (FIG. 3B). Both CD4⁺ and CD8⁺ EDA CAR-T but not PSMA CAR-T cells were able to interact with both human and mouse recombinant EDA proteins, demonstrating the specificity of the EDA CAR (FIG. 3C).

To evaluate the functionality of the CAR construct, EDA CAR-T cells were cultured in EDA coated plates for 48 h. Both CD4+ and CD8+ EDA CAR-T, but not PSMA CAR-T cells produced high amounts of IFN-γ (FIG. 3D) and proliferated in response to EDA (FIG. 3E). Interestingly, there was an apparent inverse correlation between EDA binding capacity (FIG. 3C) and functional responses by EDA CAR-T cells to mouse and human EDAs (FIGS. 3D and 3E). This discrepancy could be due to the methodological differences between assays. Indeed, in the binding experiments EDA proteins are added in solution and bind to the CAR-T cells directly. However, in the functional experiments (IFN-γ production or CAR-T cell proliferation), the EDA proteins are coated into the plate to allow the CAR crosslinking. Using Bio-Layer Interferometry (BLI) assays we found that although scFv F8 binds with similar KD for both proteins in solution, the human EDA is recognized with a slightly higher affinity than the murine EDA (7.763×10⁻⁸ M versus 9.954×10⁻⁸ M respectively). However, scFv F8 recognizes better to murine EDA protein when it is coated to ELISA plates (FIG. 13). These findings might explain why the EDA CAR-T cells bind better the human EDA when it is in solution (FIG. 3C) whereas they produce more IFN-γ in response to murine EDA when it is coated in plastic plates (FIGS. 3D and 3E).

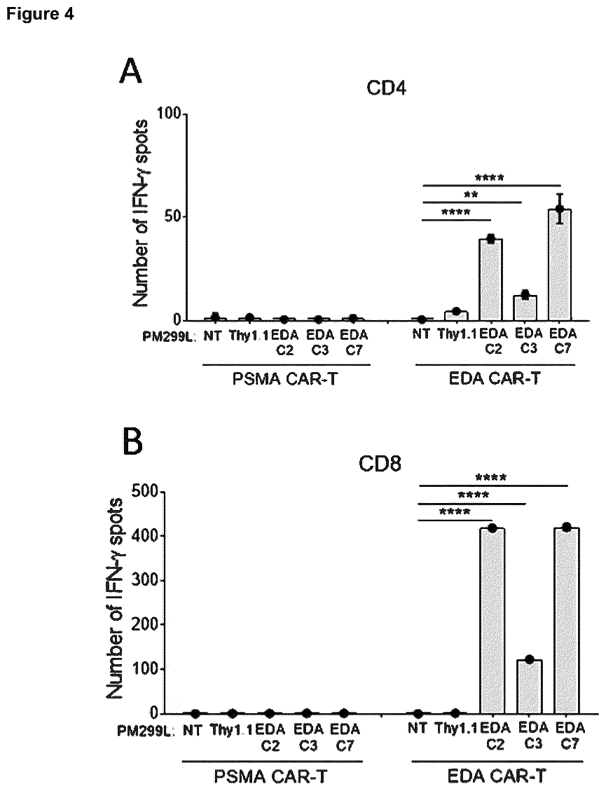

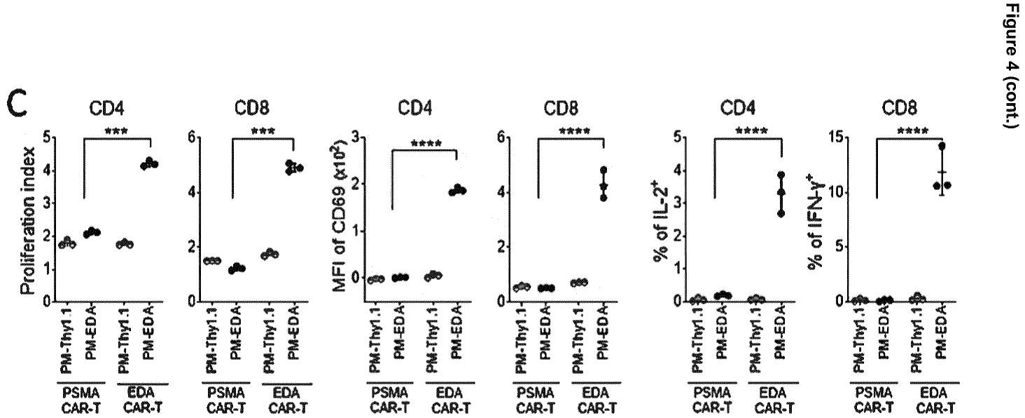

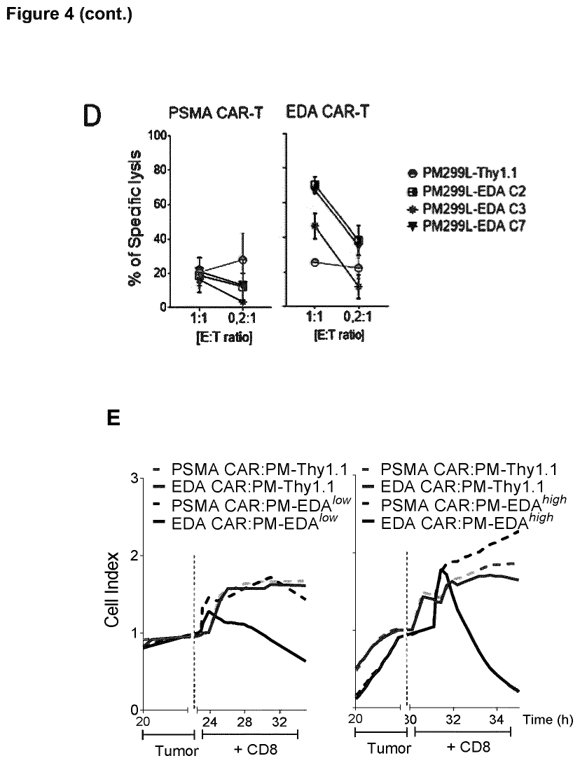

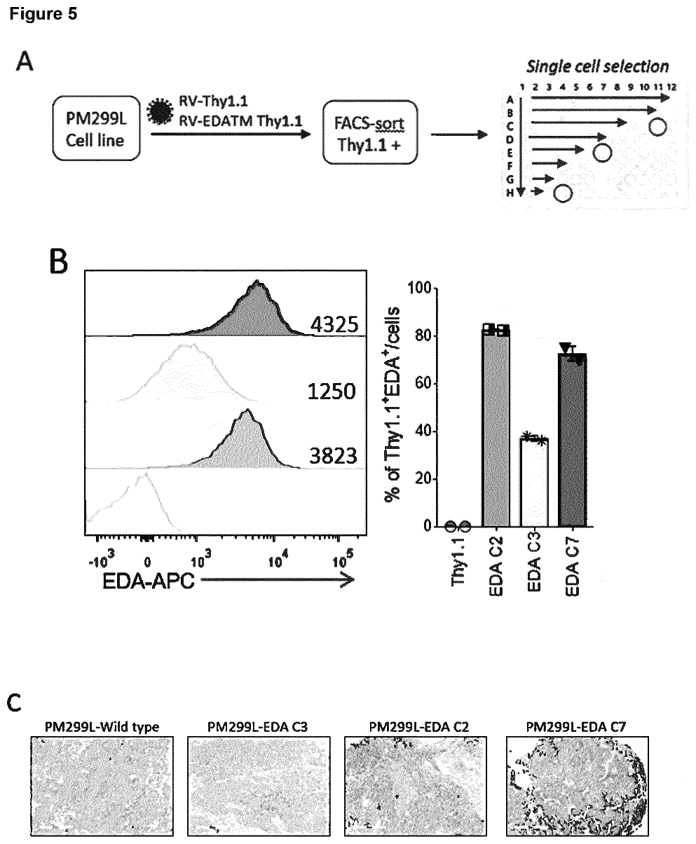

To study the capacity of EDA CAR-T cells to recognize tumor cells expressing EDA, we used the PM299L hepatocellular carcinoma cell clones C2, C3 and C7, expressing different levels of EDA on the cell membrane (FIGS. 5A and 5B). EDA CAR-T cells produced high levels of IFN-γ in response to the different PM299L cells clones. As expected, PM299L-EDA clones 2 and 7, which express high levels of the antigen, stimulated the secretion of higher levels of IFN-γ by both CD4+ and CD8+ EDA CAR-T cells than those induced by clone 3. Control PSMA CAR-T cells did not react against these cell lines indicating the specificity of EDA recognition only by EDA CAR-T cells (FIGS. 4A and 4B). T cell proliferation, CD69 expression and the release of IL-2 and IFN-γ cytokines by CD4 or CD8 EDA CAR-T cells in response to stimulation with PM299L-EDA clone 7, indicated that EDA CAR is antigen specific and efficiently triggers T cell activation (FIG. 4C).

The tumor killing capacity of EDA CAR-T cells against the different EDA-expressing PM299L clones was measured by flow cytometry (FIG. 4D). EDA CAR-T cells lysed with higher efficiency the PM299L-EDA cell clones C2 and C7 than the clone C3. However, PSMA CAR-T cells did not recognize these cell clones (left panels in FIG. 4D). Similar results were found in the real-time cytotoxicity assay (xCELLigence) (FIG. 4E). The PM299L-Thy1.1cell line, that does not express EDA, was used as control. Percentage of cell lysis was proportional to the level of EDA expression by PM299L cells (FIG. 4D, right panels).

### EXAMPLE 4. EDA CAR-T Cells Exerts Anti-Tumor Activity in Different Murine Tumor Models.

To evaluate the antitumor activity of EDA CAR-T cells we used different murine tumor models.

Example 4.1—Challenge of Immunocompetent C57BL/6 Mice Model with EDA-Expressing Transgenic PM299L-C2 Cells

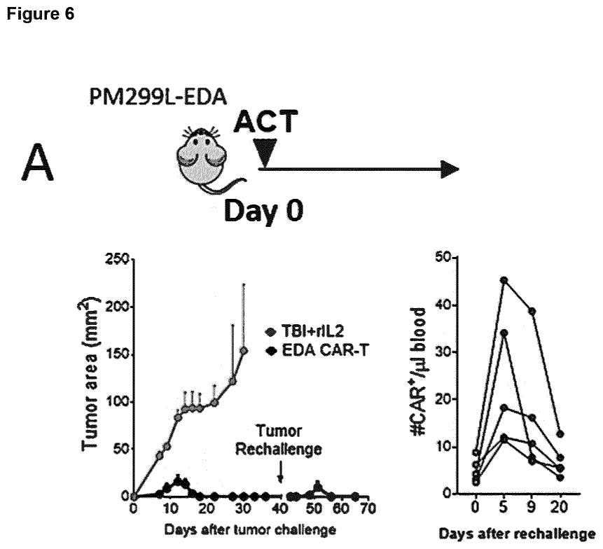

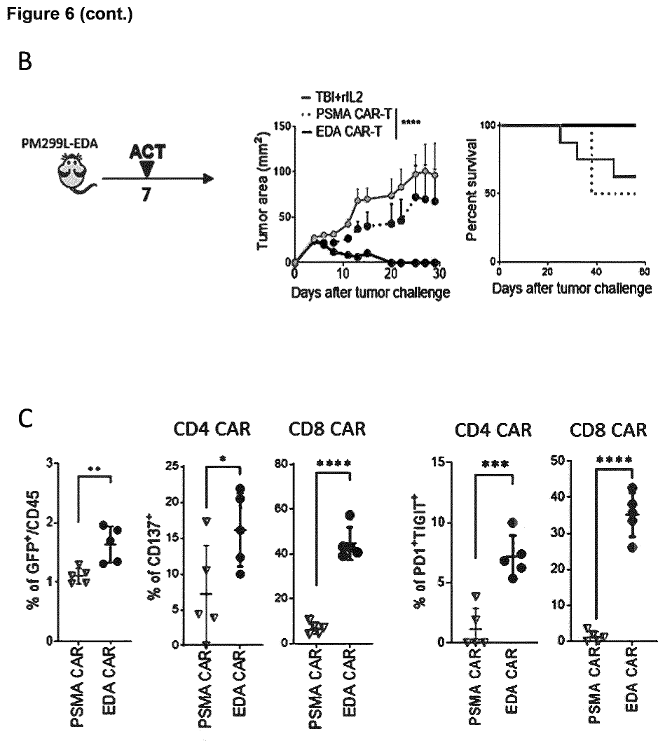

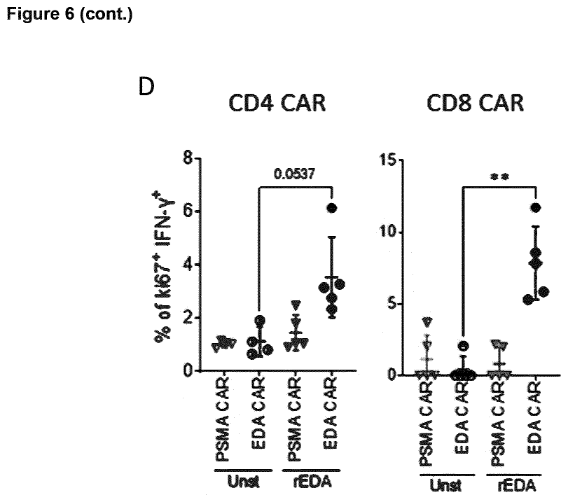

First, the antitumor activity of EDA CAR-T cells was tested in a Winn type assay (Winn H J. et al. 1961) where C57BL/6 mice were challenged subcutaneously with 2.5×10⁶ PM299L-EDA expressing cells (clone C2) (Supplementary FIGS. 5B and 5C respectively) and treated intravenously at the same time with 2.5×10⁶ CD4⁺ and 6×10⁶ CD8⁺ EDA CAR-T or PSMA CAR-T cells. While all mice treated with PSMA CAR-T cells developed tumors, administration of EDA CAR-T cells led to rejection of injected tumor cells and all animals survived to tumor challenge (FIG. 6A, left panel). These surviving EDA CAR T cell treated mice were re-challenged at day 40 with PM299L-EDA tumor cells and blood samples were obtained at different time points to evaluate by flow cytometry the EDA CAR-T cell expansion. We found a clear CAR T cell expansion five days after tumor re-challenge. The EDA CAR T numbers dropped to basal levels at day 20 after this re-challenge (FIG. 6A, right panel). All mice remained tumor free and no signs of toxicity were observed. These results suggest the establishment of a long-lasting immunity that might protect mice from metastatic recurrence.

Example 4.2—Challenge of Immunocompetent C57BL/6 Mice Model with EDA-Expressing Transgenic PM299L-C7 Cells

Second, C57BL/6J mice were challenged with PM299L-C7 cells (2.5×10⁶ cells) expressing high levels of EDA in vitro and in vivo (FIGS. 5B and 5C respectively). Seven days later, when tumors were palpable, the mice were treated with a mixture of 7×10⁶ CD4⁺ and 2×10⁶ CD8⁺ EDA CAR-T or PSMA CAR-T cells. All mice treated with EDA CAR-T, but not with PSMA CAR-T cells, rejected the tumor (FIG. 6B).

Four days after T cell therapy, mice treated with EDA CAR-T showed a significantly higher number of intratumoral CAR-T cells, with a higher level of activation (measured as the percentage of CD4⁺ or CD8⁺ CD137⁺ cells), but also a higher percentage of PD1⁺ or TIGIT⁺ T cells than those treated with PSMA CAR-T (FIG. 6C). Characterization of the functionality of CAR-T cells present in the spleen also showed a higher percentage of proliferating (Ki67⁺ IFN-γ⁺) CD4⁺ and CD8⁺ EDA CAR-T in response to EDA stimulation (FIG. 6D).

We evaluated the migratory capacity CAR-T to the site of antigen expression in mice bearing the EDA expressing PM299L-EDA C7 (injected into the right flank) and the original PM299L tumor cell line (EDA negative) (injected into the left flank). CD4⁺ and CD8⁺ EDA CAR-T or PSMA CAR-T cells were injected intravenously and 7 days after infusion, both tumors were excised to analyze the number of CAR-T cells in each one. Notably, we found that EDA CAR-T cells were enriched in the PM299L-EDA C7 tumor as compared to that found in the PM299L-derived tumor (measured as the number of CAR-T cells/mg of tumor). There was also an increase in the percentage of CAR-T cells expressing PD-1 within the EDA-expressing tumor. Since PD1 expression can be considered as an activation marker, these results suggest that EDA-CAR-T cells have been activated after antigen recognition in the tumor. However, PSMA CAR-T cells poorly infiltrated the tumors and showed no preference for any of them (FIG. 6E).

Example 4.3—Challenge of Immunocompetent Mice Model with Naturally EDA-Expressing Murine F9 Teratocarcinoma Cells

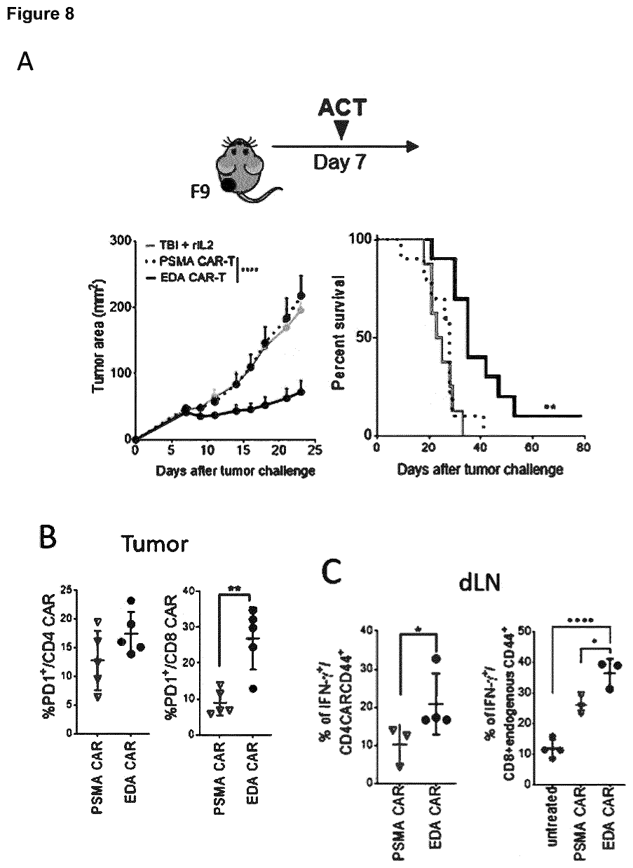

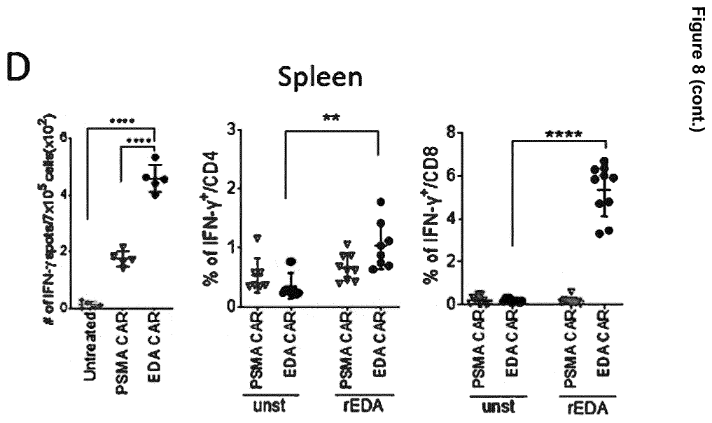

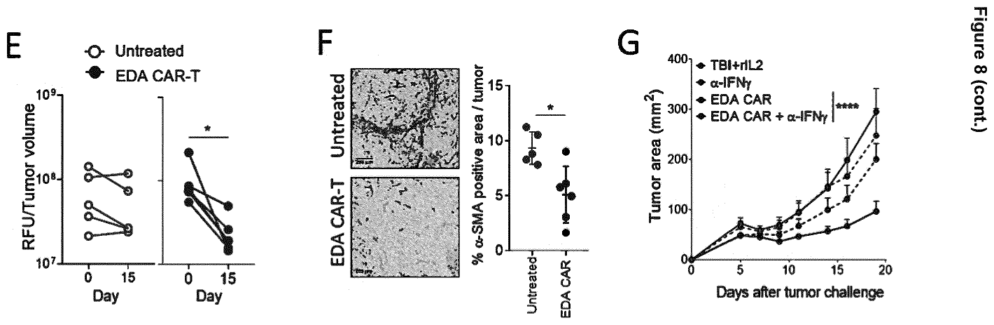

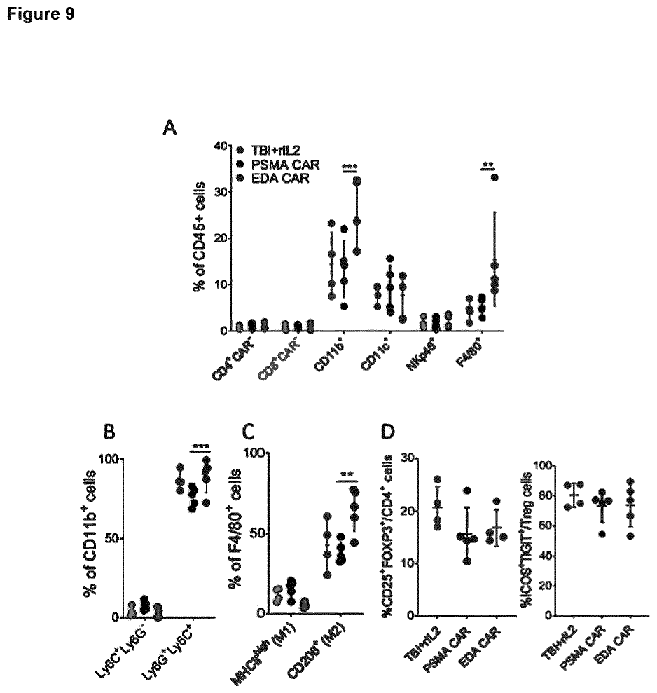

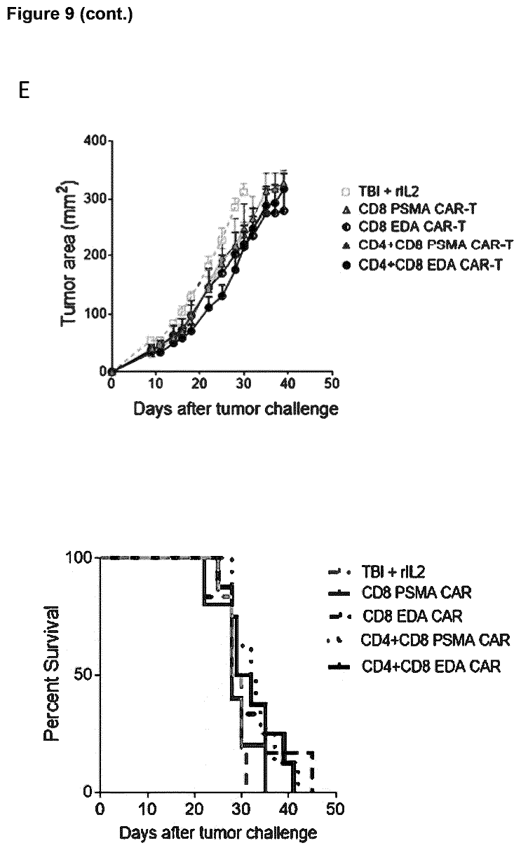

We then wanted to evaluate the antitumor capacity of EDA CAR-T cells in a tumor not forced to express EDA by genetic modification. Among B16-OVA, LLC-OVA or F9 induced tumors only the F9 teratocarcinoma tumor model expressed detectable levels of EDA (FIG. 2B). In this F9 cell-based tumor model, Rybak et al. 2007 described that the cells expressing EDA are the endogenous endothelial cells and not the tumor cells. To confirm these findings, we carried out CD31 and EDA co-localization experiments in F9 tumors as well as in B16-OVA. Interestingly, EDA expression co-localized with CD31 in F9 tumors, however, we could not detect EDA in B16 tumors, not in the tumor stroma nor in the tumor vasculature (FIG. 2C). Then, we tested the antitumor efficacy of EDA CAR-T in animals bearing F9 teratocarcinoma. 129Sv mice were challenged with F9 tumor cells (2×10⁶ cells injected by the s.c. route). When tumors were palpable, mice were treated with EDA CAR-T or PSMA CAR-T cells (a mixture of 1×10⁷ CD4⁺ and 2×10⁶ CD8⁺ CAR-T cells). It was observed that, although tumors were not totally controlled, administration of EDA CAR-T cells was able to significantly delay tumor growth (FIG. 8A). Characterization of CAR-T cells in F9 tumor-bearing mice, 7 days after cell transfer, indicated a higher percentage of PD1+ cells among CD4 or CD8 EDA CAR infiltrating the tumor with respect to PSMA CAR-T cells, although this difference was not statistically significant for CD4 EDA CAR T cells (FIG. 8B). Mice treated with EDA CAR-T cells also had a higher percentage of IFN-γ producing CD4 or CD8 CAR-T cells in the draining lymph nodes and in the spleen (FIGS. 8C and 8D respectively). Regarding the intra tumor leukocyte infiltration we found a significant increase in the percentage of CD11b+ cells, in particular the Ly6G+Ly6C+ subtype, and the F4/80+ cells, in particular the CD206+ (M2) subtype) in mice treated with EDA-CART. No significant changes were observed regarding the number of intratumor Treg cells or the endogenous CD4 or CD8 T cells (FIGS. 9A-D). The antitumor effect of EDA CAR-T cell administration was not observed in C57BL/6 mice bearing B16OVA tumors (negative for EDA staining) (FIG. 9E).

To deepen into this antitumor activity, we studied the effect of EDA-CAR therapy in the tumor microenvironment. Since in this model, EDA is expressed close to the tumor endothelium (FIG. 2C), we evaluated the effect of EDA-CART on the tumor vasculature by using AngioSense, a fluorescent probe designed to evaluate tumor vascular changes and blood vessel density in vivo. Tumor vasculature was measured in mice bearing F9 tumors before and 14 days after the adoptive transfer of EDA-CART cells. Quantitative relative fluorescence units (RFU) divided by tumor volume was analysed for each animal at these two time points. Importantly, it was found that while no significant changes were observed in the intratumor fluorescence accumulation in untreated mice, a significant reduction in fluorescence was found in those mice treated with EDA.CART cells, indicating that EDA-CART cells exerted an antiangiogenic activity (FIG. 8E). We also found a significant reduction on alpha-SMA staining in tumor samples of mice treated with EDA CAR T cells, supporting the potential impact of EDA targeting on myofibroblast differentiation and tumor stromal density (FIG. 8F). Since IFN-γ is one of the major antifibrotic factors (van Dijk F et al. 2015) and display antiangiogenic activity (Coughlin C M et al. 1998), we carried out a new in vivo assay to evaluate the antitumor activity of EDA-CART when mice were also treated with neutralizing anti-IFN-γ antibodies (i.p. injection of 100 μg/mouse at days 1, 4, 8 and 12 after EDA-CART transfer). This treatment significantly abrogated the antitumor activity of EDA-CART adoptive transfer (FIG. 8G).

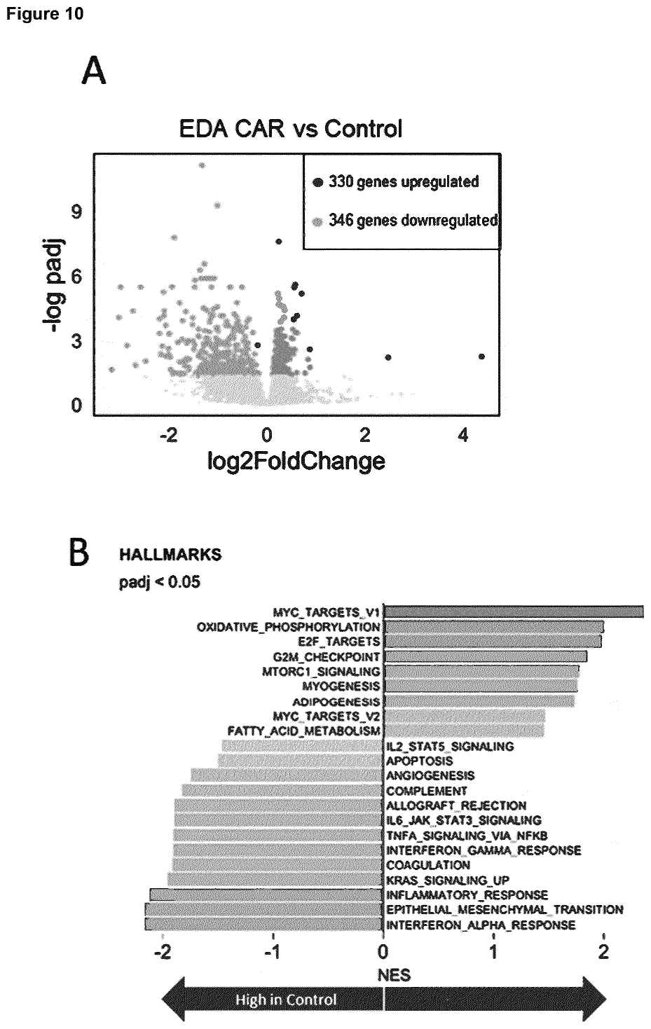

To better characterize the tumor microenviroment after adoptive cell therapy, we carried out a transcriptomic analysis of tumor tissues isolated from untreated mice and from mice treated with EDA CART cells (14 days after adoptive transfer). We found 330 genes upregulated and 346 genes dowregulated in tumors treated with EDA-CART as compared to untreated tumors (FIG. 10A). In agreement with the results obtained in the immunohistochemical analyses, GSEA enrichment analysis when compared EDA CAR treated versus untreated mice showed a significant negative enrichment in gene sets defining epithelial-mesenchymal transition (systematic name: M5930), genes encoding collagen proteins (M3005) or genes up-regulated during formation of blood vessels (angiogenesis; M5944). We also found a significant negative enrichment in gene sets defining inflammatory processes, including 1I2-STAT5, IL6-JAK-STAT3. TNF-α, IFN-γ, IFN-α or KRAS signaling, suggesting a great impact on the tumor inflammatory profile after EDA-CART transfer (FIGS. 10B and 10C).

### EXAMPLE 5. Safety of EDA-CART Cells

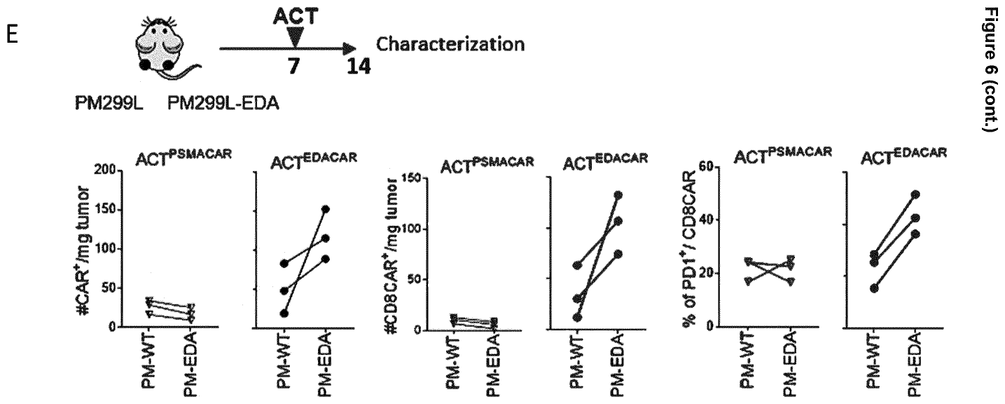

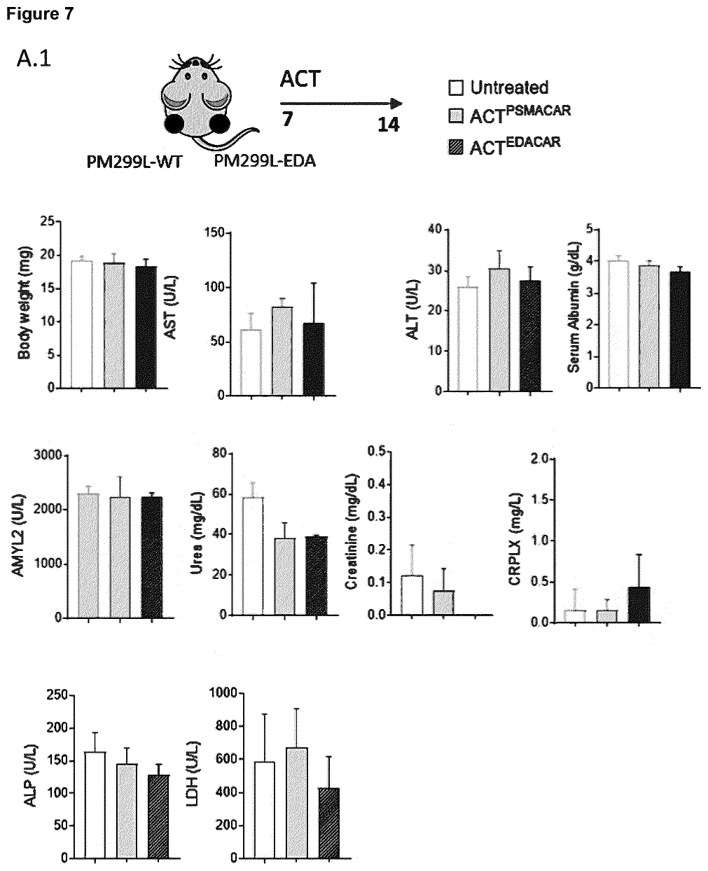

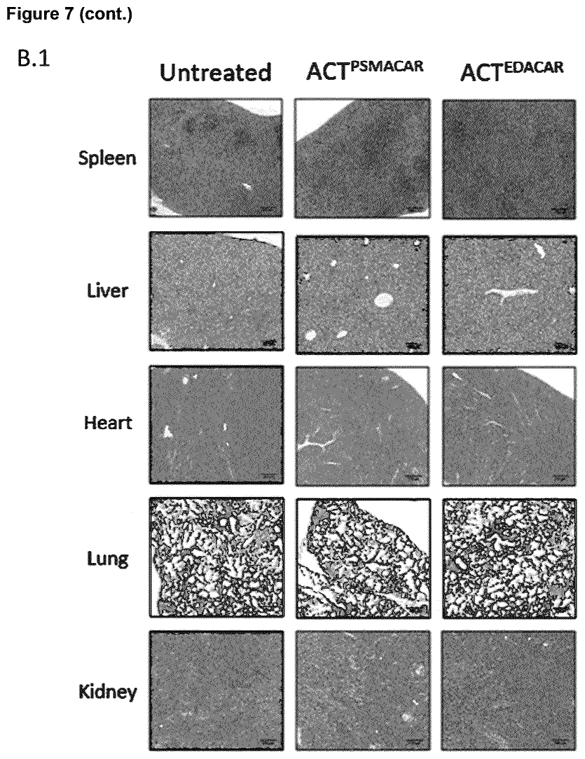

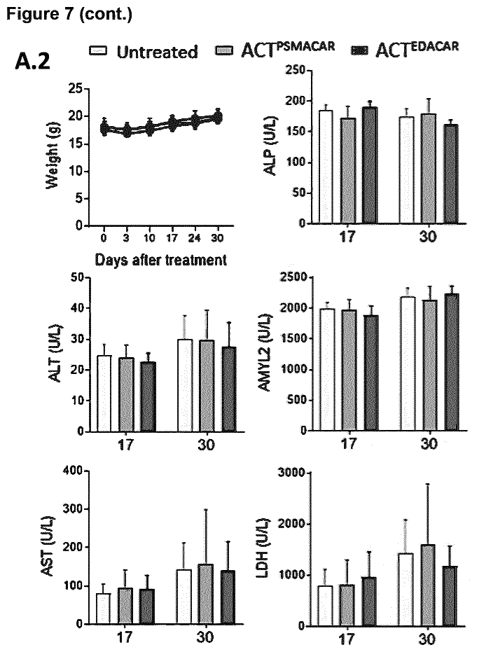

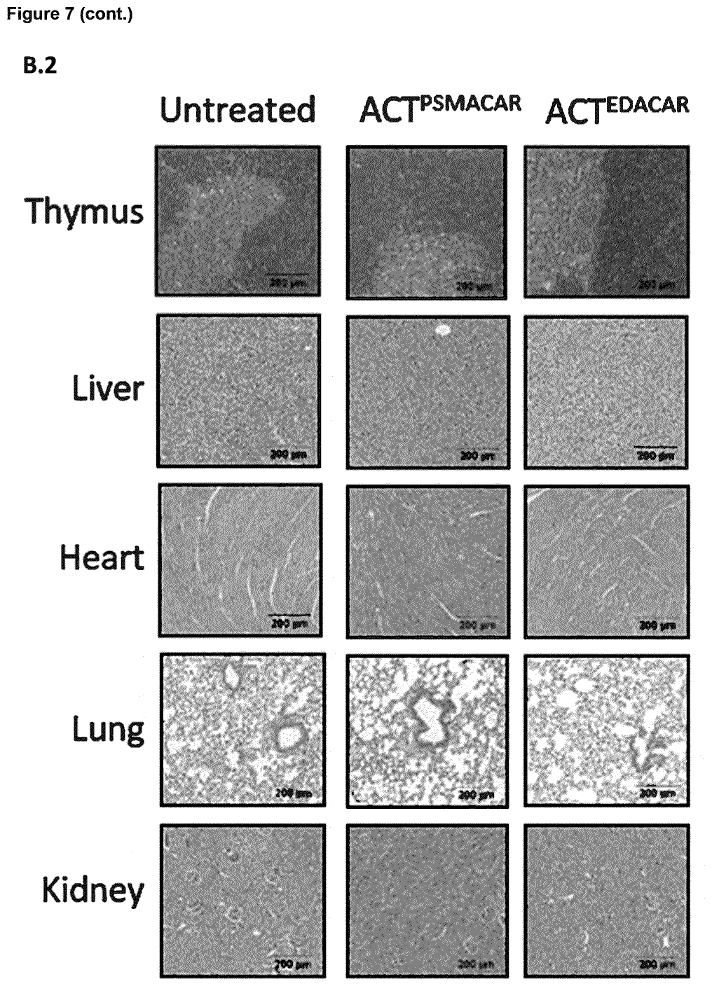

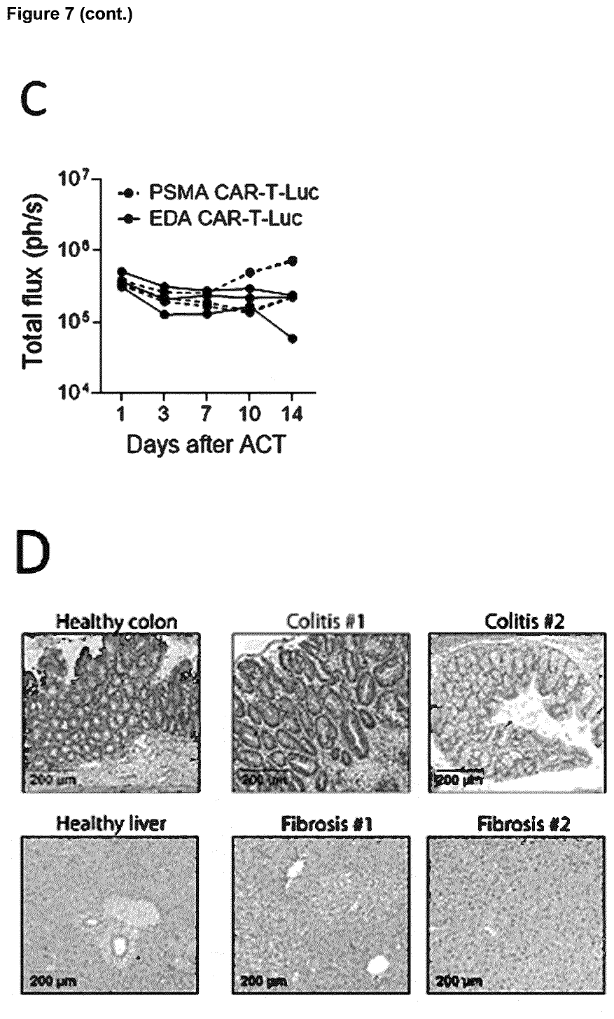

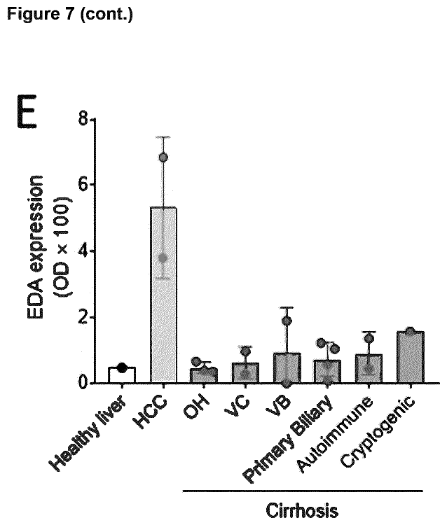

Remarkably, the injection of 1×10⁷ EDA CAR-T cells did not result in apparent toxicity for the mice. We detected no weight loss during the follow up after ACT. On day 14 after infusion, no significant changes between groups were found for AST, ALT, serum albumin, AMYL2, urea, creatinine, CRPLX, ALP or LDH levels (FIG. 7A.1) and no histological differences were observed between tissues examined from both groups (FIG. 7B.1), suggesting that EDA CAR-T cell therapy is safe.

To further assess the safety of EDA-CART, non-bearing tumor mice were injected with 1×10⁷ EDA CAR-T cells. We conducted a follow up experiment where body weight was measured periodically during all the follow up and serum samples were analyzed at day 17 and day 30 to measure biochemical parameters. We detected no weight loss during 30 days of follow up after ACT. No significant changes between groups were found for AST, ALT, serum albumin, AMYL2, urea, creatinine, CRPLX, ALP or LDH levels at days 17 and 30 after T cell infusion (FIG. 7A.2) and no histological differences were observed between tissues examined from both groups at day 30 (FIG. 7B.2).

We also evaluated the potential expansion of EDA-CART cells in non-tumor-bearing mice that received a single i.v. dose of 10⁷ EDA-CAR T cells or PSMA-CART expressing Luciferase as a reporter gene. In vivo bioluminescence was visualized using the PhotonImager Optima at days 1, 3, 7, 10 and 14 after T cell infusion. No specific expansion of EDA CAR-T lymphocytes was observed in mice at any of the times studied, suggesting that EDA-CART cells do not detect EDA antigen in a healthy mouse (FIG. 7C). Then, we studied the EDA expression in two pathogenic conditions such as an animal model of liver fibrosis and a model of inflammatory colitis. Liver samples from mice with liver fibrosis induced by CCL4 treatment, or colon samples from mice with DSS-induced colitis were stained with F8 anti EDA ScFv. The corresponding tissue sections from untreated healthy mice were used as controls. EDA expression was not detected in these pathological conditions (FIG. 7D).

We also analysed the expression of EDA in human liver samples with different types of liver cirrhosis. We included 3 samples from patients with alcoholic cirrhosis, 2 patients with HCV and 2 HBV related cirrhosis, 4 primary biliary cirrhosis, 2 autoimmune and 1 cryptogenic cirrhosis. As control, we included 1 healthy liver sample and two patients with hepatocellular carcinoma. It was found that none of these situations showed a remarkable expression of EDA, compared with the EDA expression observed in hepatocarcinoma (FIG. 7E), indicating that EDA expression is very restricted in adult tissues and suggesting that EDA CAR-T cell therapy could be safe.

### EXAMPLE 6. EDA CAR-T Cells Exerts Anti-Tumor Activity in a Xenograft Mouse Model of Human Hepatocarcinoma.

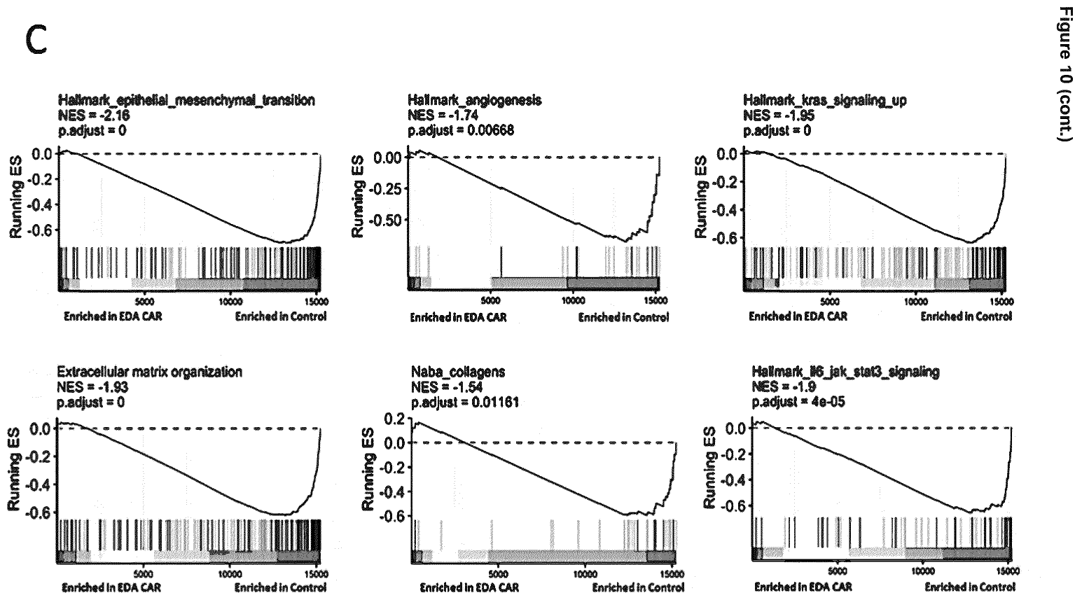

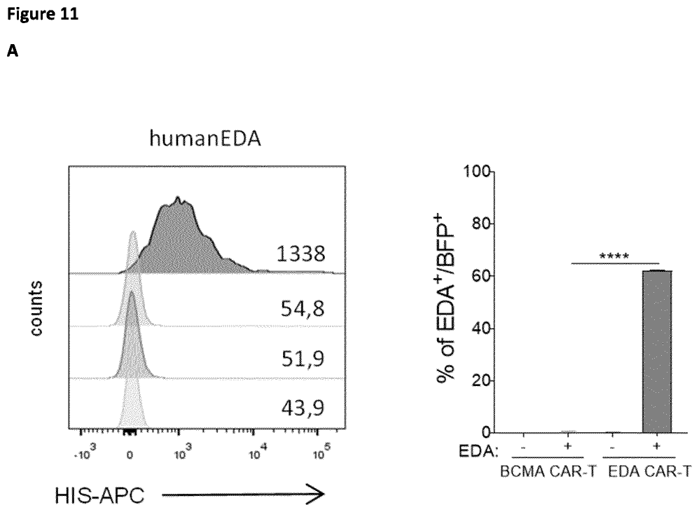

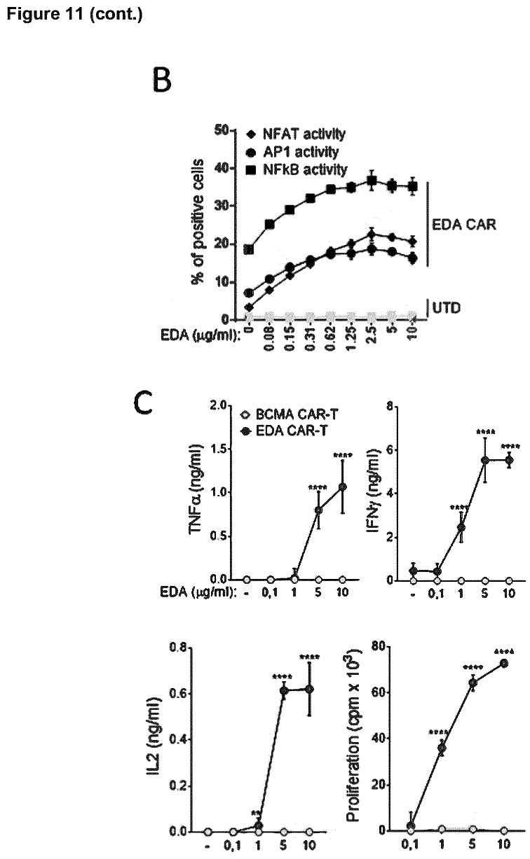

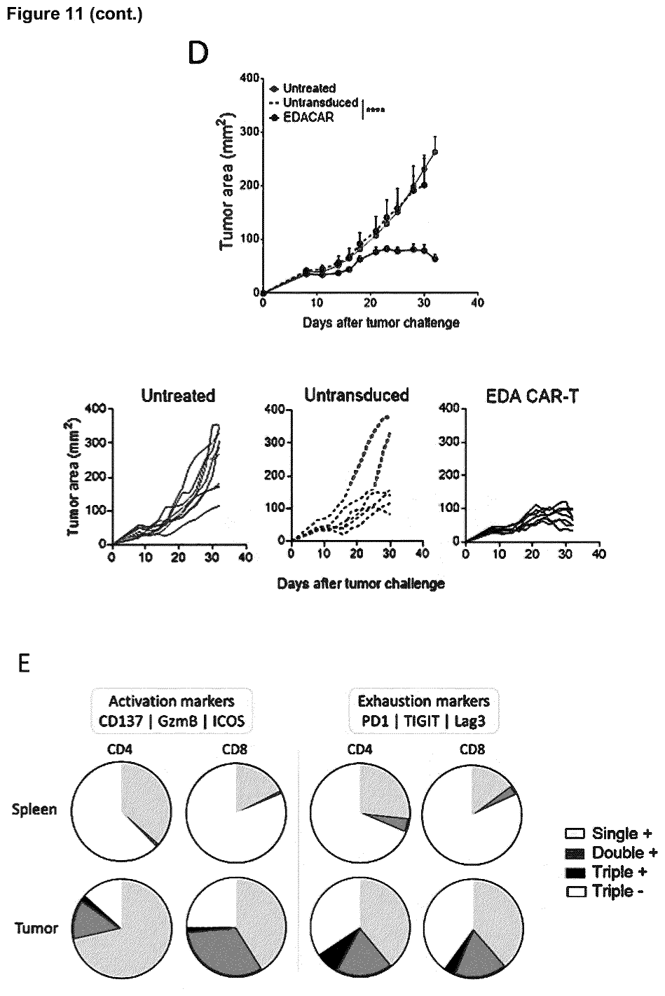

We generated a lentiviral construct encoding the EDA CAR consisting of the anti EDA F8 scFv, a short hinge/CD8 transmembrane domain, and the human 41BB and the CD3ζ endodomains. An anti-human BCMA CAR was prepared using the same backbone and used as an irrelevant CAR construct. Human EDA CAR-T cells generated by lentiviral transduction of T cells purified from healthy donors express the EDA CAR on their surface and bind human EDA-His protein (FIG. 11A). The triple parameter T cell reporter Jurkat cell line transduced with the LV expressing human EDA CAR activated NFAT, AP1 and NF-kB transcription factors when they were stimulated with EDA-coated plates (FIG. 11B). In agreement with these data, we also observed that EDA CAR-T cells produced TNF-α, IFN-γ and IL-2 and proliferated in response to increasing amounts of EDA (FIG. 11C).

The human hepatocarcinoma cell line PLC xenografted in NSG mice expressed high levels of EDA in the tumor stroma and in the CD31-expressing endothelial cells (FIG. 1G). To evaluate if the human EDA CAR-T cells could exert an antitumoral effect in this tumor model, NSG mice xenografted with PLC cell line were treated with 5×10⁶ CAR-T cells, with 5×10⁶ untransduced T cells or left untreated. Importantly, mice treated with EDA CAR were able to stop tumor growth until the end of the experiment at day 35 (FIG. 11D). In a parallel experiment, we characterized the phenotype of the EDA CAR-T cells 8 days after the T cell infusion. CAR-T cells present in the spleen and into the tumor were analyzed by flow cytometry for the expression of activation (CD137, Gzmb and ICOS) and exhaustion (PD1, TIGIT and Lag3) markers. There was a significant increase in the percentage of cells expressing one, two or three activation markers in CAR T cell isolated from the tumor with respect to those isolated from the spleen, suggesting that the antigen encounter into the tumor activated the CAR T cells. Notably, these tumor infiltrating EDA CAR-T cells also expressed exhaustion markers (FIG. 11E).

### EXAMPLE 7. EDA Protein Binds to the Cell Surface of Wild Type Jurkat Cells and Activate EDA CAR-T Cells.

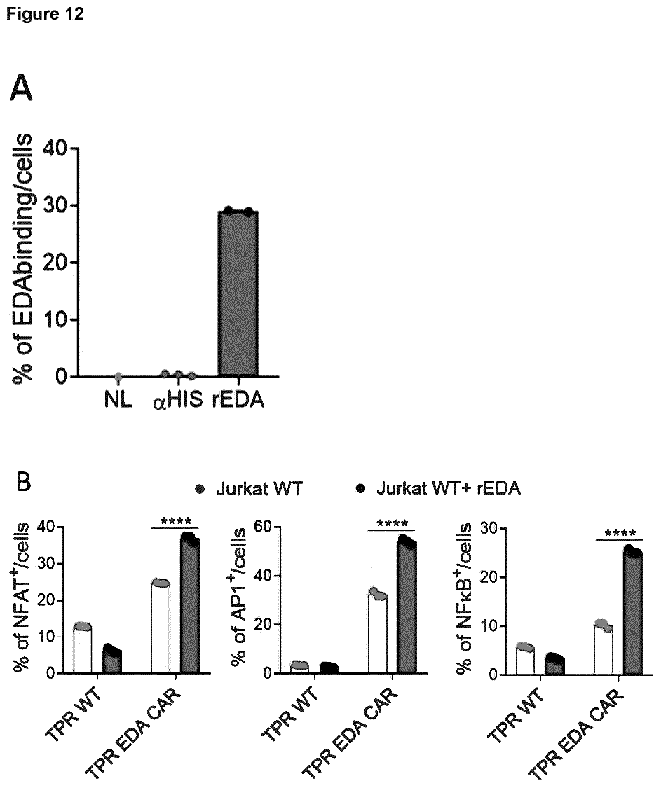

We confirmed that EDA protein binds to the cell surface of wild type Jurkat cells. Indeed, anti-His antibodies were able to bind to the His tag presented in the EDA protein coated to the wild type jurkat cells (FIG. 12; Panel A). We then tested if these wild type Jurkat cells previously incubated with soluble EDA protein could activate the NFAT, AP1 or NF-kB signaling in the TPR Jurkat cells transduced with EDA CAR. Using this reporter assay we found a significant activation of NFAT, AP1 and KF-kB in TPR reporter cells in the presence of EDA-coated Jurkat cells. Indeed, while wild type Jurkat cells did not stimulate. NFAT, NFkB and AP1 pathways in TPR wild type cells, EDA-coated Jurkat wild type cells, stimulated the NFAT, NFkB and AP1 pathways in TPR jurkat cells expressing the EDA CAR (FIG. 12; Panel B) These data supports a potential indirect recognition of cells able to bind EDA produced within the tumor.

### Discussion

The excessive deposition of collagen and FN around tumor islets significantly impair T cell motility and tumor infiltration (Salmon H et al. 2012; Joyce J A et al. 2015) by acting as a shield between T cells and tumor cells. EDA-containing FN plays a relevant role in collagen deposition and α-SMA expression by myofibroblasts, probably by its direct implication in latent TGF-β activation (Mauro A F et al. 2007) The presence of EDA highly correlated with enhanced matrix remodeling and re-organization of the actin cytoskeleton (Saito S et al. 1999), pointing toward a pro-fibrotic role for EDA in the tumor ECM.

We have demonstrated that EDA CAR-T cells can recognize EDA-expressing tumor cells and prevent tumor growth in vivo. But notably, EDA CAR-T cell infusion showed antitumor therapeutic efficacy against the challenge with tumor cells not expressing EDA. The CD31/EDA co-localization experiments suggest a localization of EDA in the tumor extracellular matrix and in the basement membrane surrounding and supporting the tumor endothelial cells. The antitumor activity of the EDA-CAR cells of the present invention seems to be mediated by IFN-γ. This cytokine might have a direct effects on tumor cells by (i) inhibiting cell proliferation or sensitizing cells to apoptosis (Langaas V et al. 2002; Mazzolini G et al. 2003), (ii) up-regulating MHC class I expression and thereby increasing tumor cell lysis by endogenous antitumor T cells (Dighe A S et al. 1994); (iii) stimulating NK activity (Brunda M J et al. 1984), or (iv) inhibiting angiogenesis (Qin Z et al. 2003). We did not found any inhibitory effect of IFN-γ on proliferation of F9 tumor cells in vitro even at doses of 5000 U/ml. Up-regulation of MHC class I molecules and the susceptibility to CTL-dependent lysis or the activation of NK cells may not be the main mechanisms since we did not found a significant increase of endogenous T cells after EDA-CART therapy. Moreover, EDA-CART cells also showed an antitumor effect in NSG mice challenged with human PLC tumor cell line. Thus, without willing to be bound by theory, it is proposed by the inventors that the IFN-γ-dependent antiangiogenic activity may constitute an important mechanism of action of the EDA-CART cells.

The RNAsec analysis revealed an important effect of the EDA-CAR cell therapy in the tumor microenvironment with a significant reduction in gene signatures associated with epithelial-mesenchymal transition, genes encoding collagen proteins or genes up-regulated during formation of blood vessels as well as in gene sets defining inflammatory processes. Chronic inflammation generated by the tumor microenvironment is known to drive cancer initiation, proliferation, progression, metastasis, and therapeutic resistance (Quinn K M et al. 2020). Notably, we found that EDA-CART promoted a significant reduction in the IL-6-STAT5 and the KRAS related signatures that has been associated to a poor prognosis in many cancers (Caetano M S et al. 2016; Johnson D E et al. 2018).

There is also evidence for an immunosuppressive function of EDA through the recruitment of Treg cells (Sengupta S et al. 2010) or the activation of myeloid-derived suppressor cells (Rossnagl S et al. 2016), that weaken the immune response against cancer confirming its pro-tumor effect. All these functions induced by FN-EDA might be elicited through its binding to the integrins α4β1, α4β7 (Liao Y F et al. 2002), α9β1 (Sun X et al. 2013), α5β1 (Manabe R et al. 1997) or to TLR4 (Okamura Y et al. 2001) displaying a direct protumorigenic activity (Nam J M et al. 2010; Liu W T et al. 2014). Other CAR-T cells attempt to disrupt tumor neovasculature by targeting α5β3 integrin (Fu X et al. 2013), TEM8 (Byrd T T et al. 2017; Petrovic K et al. 2019) or CLEC14A (Zhuang X et al. 2020), although some toxicity issues have been also arisen (Petrovic K et al. 2019; Chinnasamy D et al. 2010) probably because of the “on-target off-tumor” activity of the CAR-T.

It is plausible that EDA secretion by tumor cells or tumor infiltrating cells enabled EDA CAR-T cells to target tumor vasculature or tumor cells that express the potential EDA receptors such as some integrins or TLR4. We tested this possibility using the Jurkat TPR system, that allows to evaluate T cell activation primed by EDA-CAR after antigen recognition. First, we confirmed that EDA proteins binds to the cell surface of wild type Jurkat cells, which express high levels of integrins α4β1 among others (Rose D M et al. 2001). We then tested if these wild type Jurkat cells previously incubated with soluble EDA protein could activate the NFAT, AP1 or NF-kB signalling in the TPR Jurkat cells transduced with EDA-CAR. Using this reporter assay we found a significant activation of NFAT, AP1 and KF-kB in TPR reporter cells in the presence of EDA-coated Jurkat cells, supporting a potential indirect recognition of cells able to bind EDA produced within the tumor (FIG. 12).

We did not observe any clinical signs of toxicity in the EDA CAR-T cell treated animals suggesting that EDA is not expressed in healthy tissues, at least at the level needed for CAR-T cell activation, and also that EDA CAR-T cells are well-tolerated with no noticeable “on-target/off-tumor” toxicity. Human EDA CAR-T cells exerted a strong antitumoral effect in a NSG xenograft model for human hepatocarcinoma, suggesting a potential translation to human settings for the treatment of human tumors with upregulated EDA expression.

EXAMPLE 8. Comparative Example: In Vitro and In Vivo Assessment of EDA CAR-T Cells Generated with CARs Comprising F8, C27 or C33 Anti-EDA scFv.

### 1. EDA Protein Recognition by EDA CAR T Cells Bearing Different Anti EDA-ScFv.

We prepared a retroviral construct encoding a cassette for the expression of the different EDA-specific CAR cloned in the pRubiG plasmid in frame with the eGFP-P2A gene to express eGFP and the CAR simultaneously. The F8, C27 and C33 clones are characterized by comprising the VH and VL domains of the previously described F8 (Rybak J N et al. 2007), 27A12.70 and the 33E3.10 antibodies (WO2015/088348 A1). These VH and VL domains were cloned in the VH/VL orientation with a linker of SEQ ID NO: 23 in between, respectively. The nucleotide and amino acid sequences of these three scFv clones are as follows: clone F8 (SEQ ID NO: 24; SEQ ID NO: 9), clone C27 (SEQ ID NO: 25 and 26) and clone C33 (SEQ ID NO: 27 and 28)

EDA CAR-T cells were generated by retroviral transduction of CD4⁺ or CD8⁺ T cells with the corresponding retrovirus. Five days after infection, T cells were analyzed by flow cytometry. Both CD4⁺ and CD8⁺ EDA CAR-T transduced cells express their respective CAR construct with an efficiency of transduction in the 80% range in all cases. To evaluate the functionality of the CAR construct, EDA CAR-T cells were cultured in EDA coated plates for 48 h. IFN-g released to the culture supernatant was measured by ELISA. Both CD4⁺ and CD8⁺ EDA CAR-T clones F8 and C33 were induced to produce IFN-gamma in response to both human and mouse recombinant EDA proteins, demonstrating the specificity of the EDA CAR. We observed that clone C33 EDA CART was able to produce higher amounts of IFN-g in comparison with C27 and F8 clones when it was stimulated with decreasing concentrations of EDA coated to the plate; suggesting a higher affinity of clone C33 to recognize murine or human EDA. In the case of clone C27, we found that it was able to recognize human EDA with an affinity similar to that found for clone C33. However, C27 clone did not recognize murine EDA (FIG. 14).

Similar results were observed when T cell proliferation of the different EDA CART cell clones was measured by the tritiated thymidine incorporation assay in response to stimulation with murine or human EDA recombinant proteins coated plates (FIG. 15).

### 2. Recognition of Tumor Cells Expressing EDA by Anti EDA CAR T Cells Bearing Different Anti EDA-ScFv.

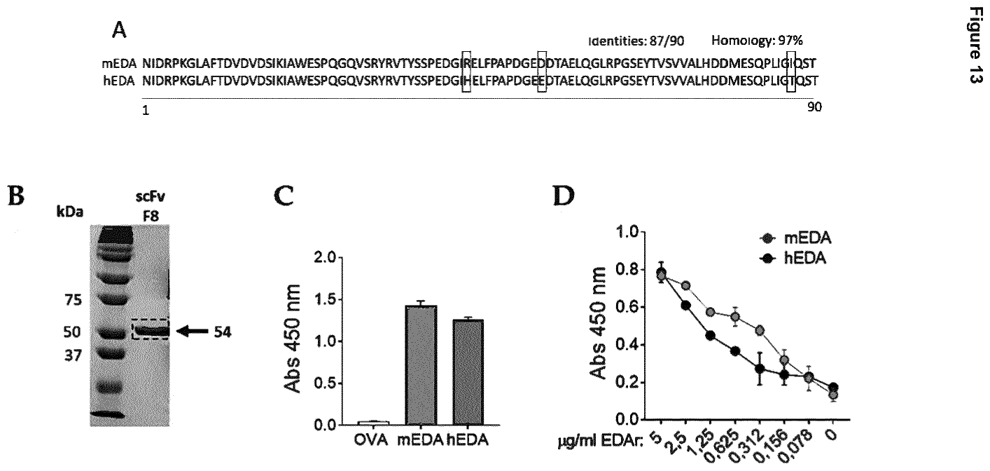

To study the capacity of EDA CAR-T cells to recognize tumor cells expressing EDA, we used the PM299L-EDA hepatocellular carcinoma cell line expressing murine EDA on the cell membrane. F8 EDA CAR-T cells produced high levels of IFN-γ in response to PM299L-EDA stimulation. However, neither the C27 EDA CAR-T nor the C33 EDA CAR-T cells were able to recognize the PM299L-EDA tumor cells. Despite F8 EDA CAR-T cells having been found to recognize the murine or human EDA protein with a lower affinity than the C33 EDA CAR-T cells, both CD4 and CD8 cells expressing the F8 EDA CAR produced high amounts of IFN-γ levels in response to PM299L-EDA (FIG. 16).

### 3. EDA CAR-T Cells Exerts Anti-Tumor Activity in Different Murine Tumor Models.

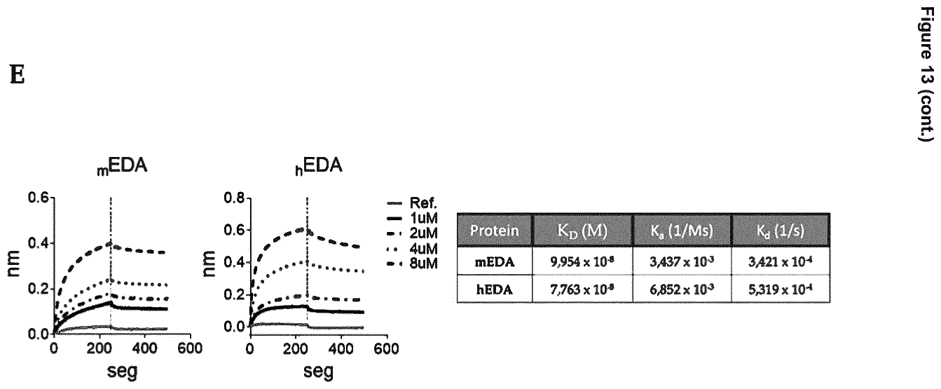

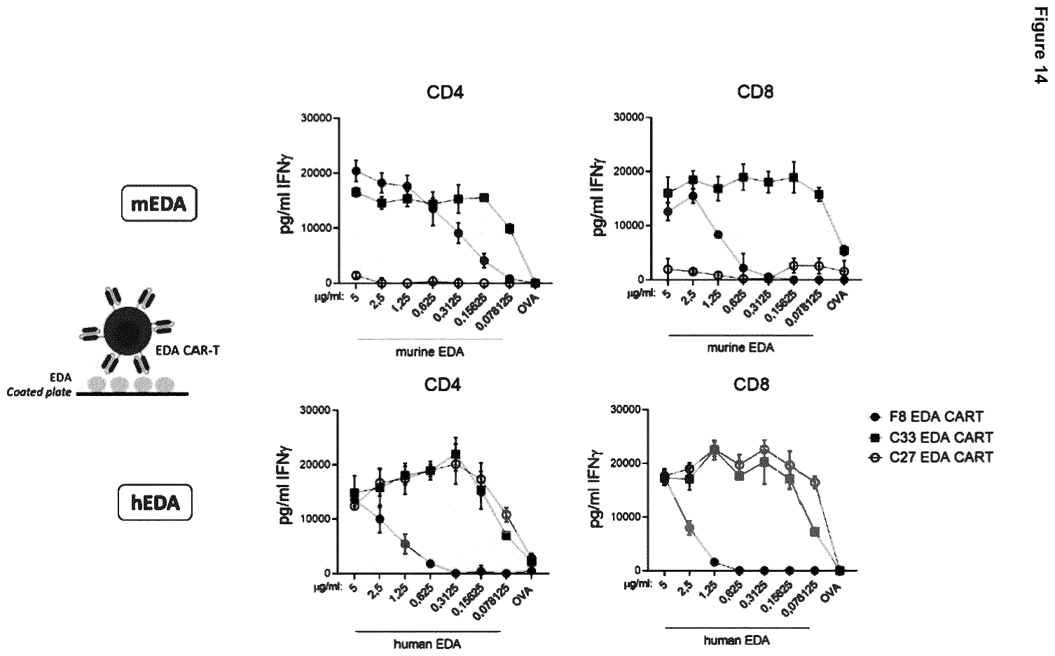

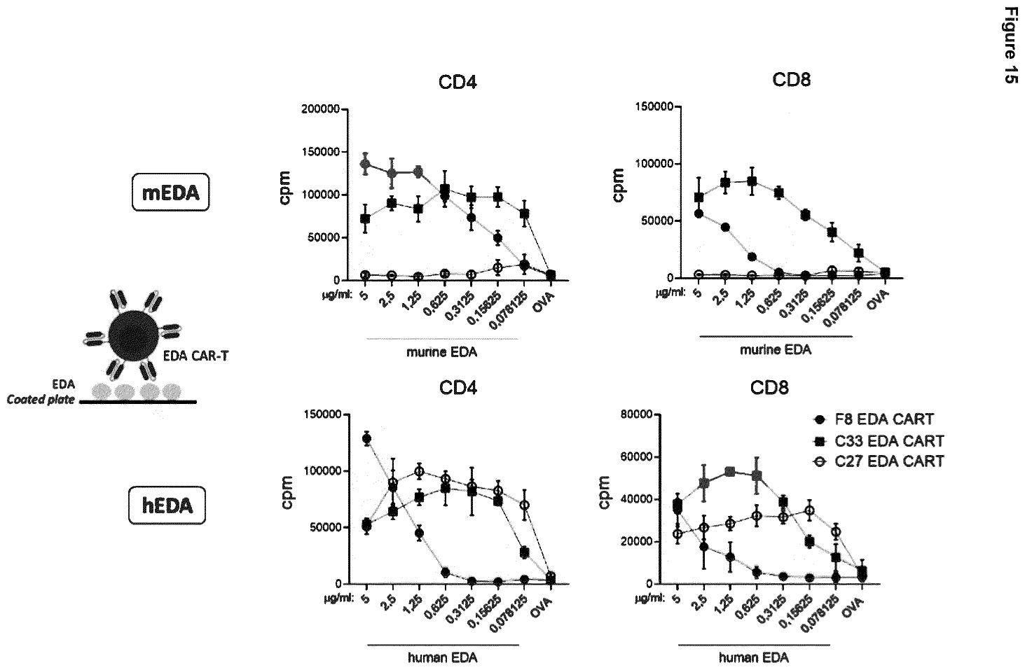

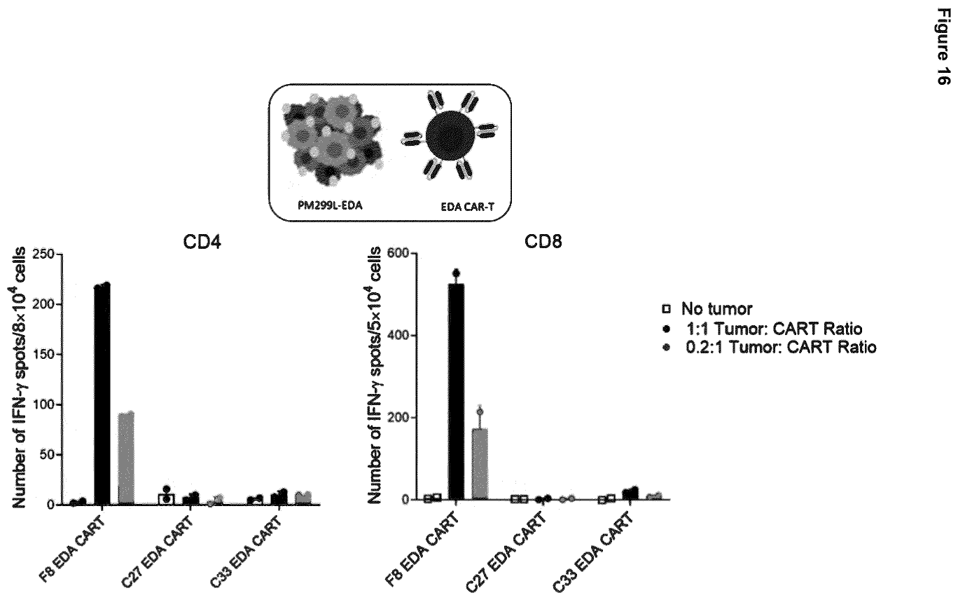

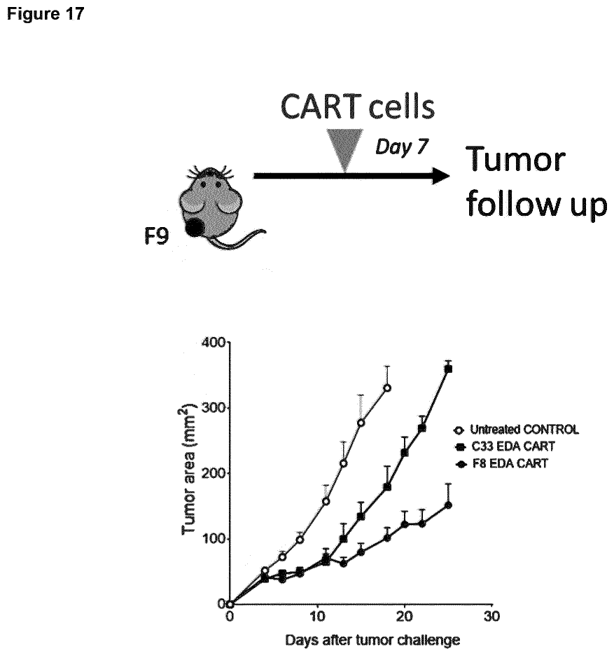

We evaluated the antitumor capacity of F8 or C33 EDA CAR-T cells in the F9 based teratocarcinoma model. C27 EDA CAR-T cells were not assayed in the mice model since these did not recognize murine EDA. In this F9 cell-based tumor model, Rybak et al (Rybak et al. 2007) described that the cells expressing EDA are the endogenous endothelial cells and not the tumor cells. We confirmed this expression in our murine model. 129Sv mice were challenged with F9 tumor cells (3×10⁶ cells injected subcutaneously). When tumors were palpable, mice were treated with EDA CAR-T (a mixture of 1×10⁷ CD4⁺ and CD8⁺ CAR-T cells). It was observed that, both F8 and C33 ECA CART cells were able exert antitumor activity delaying tumor growth. In this model, the F8 EDA CART cells showed a better antitumor activity than C33 EDA CART cells, although both EDA CART cells were effective delaying tumor growth (FIG. 17).

### EXAMPLE 9. Determination of EDA Expression in Primary and Metastatic HCC by Immunohistochemistry

We evaluated EDA expression using the F8 ScFv in a new cohort of primary hepatocellular carcinoma (HCC) as well as in a cohort of metastatic HCC by immunohistochemistry (IHC). We randomly selected 10 fields and quantified the densitometry of each of them using the ImageJ program. We considered the value of each field as positive when it exceeded the mean value+2SD of the densitometry of an immunostaining obtained in a negative control (in the absence of the F8 ScFv antibody).

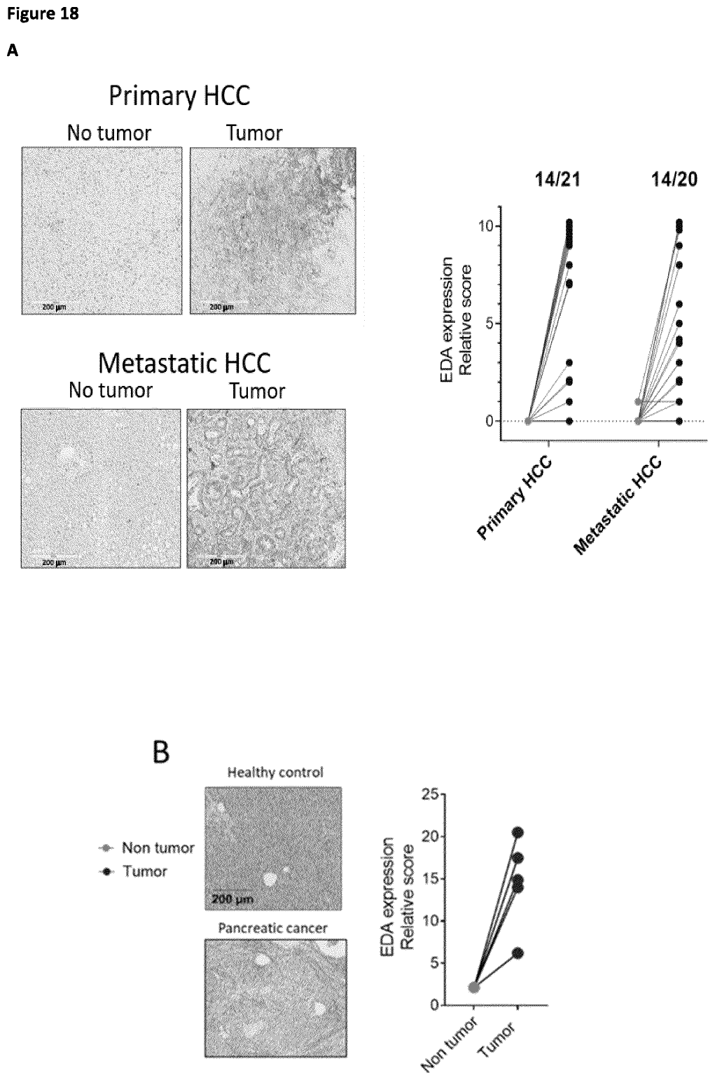

The figure shows the number of positive fields (out of a total of 10) for each patient's tumor with respect to its corresponding non-tumoral region (paired sample). A significant upregulation of EDA expression was found in the tumor tissue with respect to the surrounding normal tissue in both cohorts of patients. Similar results were found in a cohort of pancreatic ductal adenocarcinoma (PDAC) patients (FIG. 18)

EXAMPLE 10. Antitumor Effect of EDA CAR-T, EDB CAR-T or IIICS CAR-T Cells in NSG Mice Xenotransplanted with the Human Hepatocarcinoma Cell Line PLC

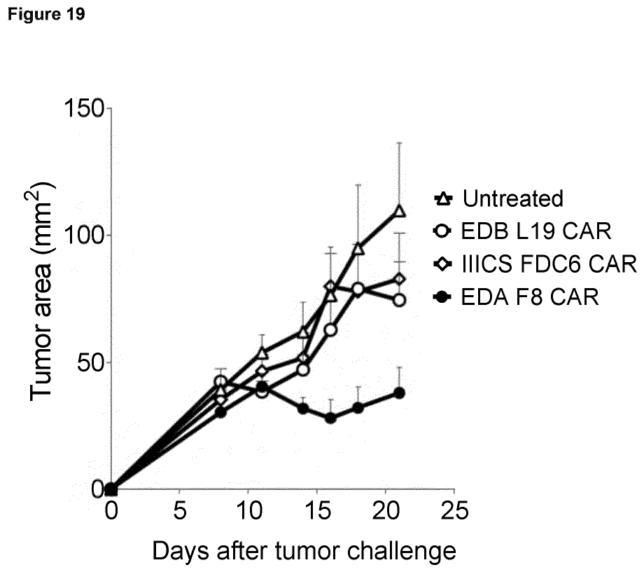

Human CART cells specific for EDA, EDB or IIICS were prepared using replication-defective lentiviral vectors comprising CARs containing a ScFv specific for EDA (F8 clone), EDB (L19 clone as described in WO 2021087356 A1) or IIICS (FDC6 clone as described in WO 2021087356 A1) domains of fibronectin. Antitumor activity of these human CAR T cells was tested in NSG mice xenotransplanted with the human hepatocarcinoma cell line PLC. NSG mice (n=5-6 mice per group) were challenged with PLC tumor cells and 8 days later, when the tumors reached 5 mm in diameter, mice were treated with 2.5×10⁶ CAR transduced T cells (CD4 and CD8 cells at a ratio 2:1) or left untreated. Mean tumor area+/−SEM at different time points for each treatment is plotted in FIG. 19. The graph illustrates that the EDA CAR T cell treatment resulted in the highest inhibition of tumor growth. Thus, showing superior tumor inhibition properties with respect to the EDB and IIICS CAR T cell treatments described in WO 2021087356 A1, in the human hepatocarcinoma (PLC) model.

### REFERENCES

1. Brentjens R J, Davila M L, Riviere I, et al. CD19-targeted T cells rapidly induce molecular remissions in adults with chemotherapy-refractory acute lymphoblastic leukemia. Sci Transl Med 2013; 5 (177): 177ra38. doi: 10.1126/scitranslmed.3005930 [published Online First: 2013 Mar. 22]
2. Grupp S A, Kalos M, Barrett D, et al. Chimeric antigen receptor-modified T cells for acute lymphoid leukemia. N Engl J Med 2013; 368 (16): 1509-18. doi: 10.1056/NEJMoa1215134 [published Online First: 2013 Mar. 27]
3. Wagner J, Wickman E, DeRenzo C, et al. CAR T Cell Therapy for Solid Tumors: Bright Future or Dark Reality? Mol Ther 2020; 28 (11): 2320-39. doi: 10.1016/j.ymthe.2020.09.015 [published Online First: 2020 Sep. 27]
4. Caruana I, Savoldo B, Hoyos V, et al. Heparanase promotes tumor infiltration and antitumor activity of CAR-redirected T lymphocytes. Nat Med 2015; 21 (5): 524-9. doi: 10.1038/nm.3833 [published Online First: 2015 Apr. 8]
5. Jiang H, Hegde S, DeNardo D G. Tumor-associated fibrosis as a regulator of tumor immunity and response to immunotherapy. Cancer Immunol Immunother 2017; 66 (8): 1037-48. doi: 10.1007/s00262-017-2003-1 [published Online First: 2017 Apr. 30]
6. Salmon H, Franciszkiewicz K, Damotte D, et al. Matrix architecture defines the preferential localization and migration of T cells into the stroma of human lung tumors. J Clin Invest 2012; 122 (3): 899-910. doi: 10.1172/JCI45817 [published Online First: 2012 Feb. 2]
7. Tahkola K, Ahtiainen M, Mecklin J P, et al. Stromal hyaluronan accumulation is associated with low immune response and poor prognosis in pancreatic cancer. Sci Rep 2021; 11 (1): 12216. doi: 10.1038/s41598-021-91796-x [published Online First: 2021 Jun. 11]
8. Efthymiou G, Saint A, Ruff M, et al. Shaping Up the Tumor Microenvironment With Cellular Fibronectin. Front Oncol 2020; 10:641. doi: 10.3389/fonc.2020.00641 [published Online First: 2020 May 20]
9. Wang J P, Hielscher A. Fibronectin: How Its Aberrant Expression in Tumors May Improve Therapeutic Targeting. J Cancer 2017; 8 (4): 674-82. doi: 10.7150/jca. 16901 [published Online First: 2017 Apr. 4]
10. Kumra H, Reinhardt D P. Fibronectin-targeted drug delivery in cancer. Adv Drug Deliv Rev 2016; 97:101-10. doi: 10.1016/j.addr.2015.11.014 [published Online First: 2015 Dec. 8]
11. Moschetta M, Pretto F, Berndt A, et al. Paclitaxel enhances therapeutic efficacy of the F8-IL2 immunocytokine to EDA-fibronectin-positive metastatic human melanoma xenografts. Cancer Res 2012; 72 (7): 1814-24. doi: 10.1158/0008-5472.CAN-11-1919 [published Online First: 2012 Mar. 7]
12. Rosini E, Volpi N A, Ziffels B, et al. An antibody-based enzymatic therapy for cancer treatment: The selective localization of D-amino acid oxidase to EDA fibronectin. Nanomedicine 2021; 36:102424. doi: 10.1016/j.nano.2021.102424 [published Online First: 2021 Jun. 27]
13. Ryan M C, Cleland J, Kim R, et al. SpliceSeq: a resource for analysis and visualization of RNA-Seq data on alternative splicing and its functional impacts. Bioinformatics 2012; 28 (18): 2385-7. doi: 10.1093/bioinformatics/bts452 [published Online First: 2012 Jul. 24]
14. Ryan M, Wong W C, Brown R, et al. TCGASpliceSeq a compendium of alternative mRNA splicing in cancer. Nucleic Acids Res 2016; 44 (D1): D1018-22. doi: 10.1093/nar/gkv1288 [published Online First: 2015 Nov. 26]
15. Jutz S, Leitner J, Schmetterer K, et al. Assessment of costimulation and coinhibition in a triple parameter T cell reporter line: Simultaneous measurement of N F-kappaB, NFAT and A P-1. J Immunol Methods 2016; 430:10-20. doi: 10.1016/j.jim.2016.01.007 [published Online First: 2016 Jan. 19]
16. Rybak J N, Roesli C, Kaspar M, et al. The extra-domain A of fibronectin is a vascular marker of solid tumors and metastases. Cancer Res 2007; 67 (22): 10948-57. doi: 10.1158/0008-5472.CAN-07-1436 [published Online First: 2007 Nov. 17]
17. Lasarte J J, Casares N, Gorraiz M, et al. The extra domain A from fibronectin targets antigens to TLR4-expressing cells and induces cytotoxic T cell responses in vivo. J Immunol 2007; 178 (2): 748-56. doi: 178/2/748 [pii] [published Online First: 2007 Jan. 5]
18. Lozano T, Chocarro S, Martin C, et al. Genetic Modification of CD8(+) T Cells to Express EGFR: Potential Application for Adoptive T Cell Therapies. Front Immunol 2019; 10:2990. doi: 10.3389/fimmu.2019.02990 [published Online First: 2020 Jan. 11]
19. Casares N, Rudilla F, Arribillaga L, et al. A peptide inhibitor of FOXP3 impairs regulatory T cell activity and improves vaccine efficacy in mice. J Immunol 2010; 185 (9): 5150-9. doi: 10.4049/jimmunol. 1001114 [published Online First: 2010 Sep. 28]
20. Balza E, Borsi L, Allemanni G, et al. Transforming growth factor beta regulates the levels of different fibronectin isoforms in normal human cultured fibroblasts. FEBS Lett 1988; 228 (1): 42-4. doi: 10.1016/0014-5793 (88) 80580-5 [published Online First: 1988 Feb. 8]
21. Ventura E, Weller M, Macnair W, et al. TGF-beta induces oncofetal fibronectin that, in turn, modulates TGF-beta superfamily signaling in endothelial cells. J Cell Sci 2018; 131 (1) doi: 10.1242/jcs.209619 [published Online First: 2017 Nov. 22]
22. Glukhova M A, Frid M G, Shekhonin B V, et al. Expression of fibronectin variants in vascular and visceral smooth muscle cells in development. Dev Biol 1990; 141 (1): 193-202. doi: 10.1016/0012-1606 (90) 90114-x [published Online First: 1990 Sep. 1]
23. Bergers G, Benjamin L E. Tumorigenesis and the angiogenic switch. Nat Rev Cancer 2003; 3 (6): 401-10. doi: 10.1038/nrc1093 [published Online First: 2003 Jun. 5]
24. Itatani Y, Kawada K, Yamamoto T, et al. Resistance to Anti-Angiogenic Therapy in Cancer-Alterations to Anti-VEGF Pathway. Int J Mol Sci 2018; 19 (4) doi: 10.3390/ijms 19041232 [published Online First: 2018 Apr. 20]
25. Dvorak H F. Tumors: wounds that do not heal. Similarities between tumor stroma generation and wound healing. N Engl J Med 1986; 315 (26): 1650-9. doi: 10.1056/NEJM198612253152606 [published Online First: 1986 Dec. 25]
26. Perryman L, Erler J T. Lysyl oxidase in cancer research. Future Oncol 2014; 10 (9): 1709-17. doi: 10.2217/fon.14.39 [published Online First: 2014 Aug. 26]
27. Keely P J. Mechanisms by which the extracellular matrix and integrin signaling act to regulate the switch between tumor suppression and tumor promotion. J Mammary Gland Biol Neoplasia 2011; 16 (3): 205-19. doi: 10.1007/s10911-011-9226-0 [published Online First: 2011 Aug. 9]
28. Werb Z, Lu P. The Role of Stroma in Tumor Development. Cancer J 2015; 21 (4): 250-3. doi: 10.1097/PPO.0000000000000127 [published Online First: 2015 Jul. 30]
29. Paszek M J, Zahir N, Johnson K R, et al. Tensional homeostasis and the malignant phenotype. Cancer Cell 2005; 8 (3): 241-54. doi: 10.1016/j.ccr.2005.08.010 [published Online First: 2005 Sep. 20]
30. Van Obberghen-Schilling E, Tucker R P, Saupe F, et al. Fibronectin and tenascin-C: accomplices in vascular morphogenesis during development and tumor growth. Int J Dev Biol 2011; 55 (4-5): 511-25. doi: 10.1387/ijdb.103243eo [published Online First: 2011 Jul. 20]
31. Joyce J A, Fearon D T. T cell exclusion, immune privilege, and the tumor microenvironment. Science 2015; 348 (6230): 74-80. doi: 10.1126/science.aaa6204 [published Online First: 2015 Apr. 4]
32. Muro A F, Moretti F A, Moore B B, et al. An essential role for fibronectin extra type III domain A in pulmonary fibrosis. Am J Respir Crit Care Med 2008; 177 (6): 638-45. doi: 10.1164/rccm.200708-1291O C [published Online First: 2007 Dec. 22]
33. Saito S, Yamaji N, Yasunaga K, et al. The fibronectin extra domain A activates matrix metalloproteinase gene expression by an interleukin-1-dependent mechanism. J Biol Chem 1999; 274 (43): 30756-63. doi: 10.1074/jbc.274.43.30756 [published Online First: 1999 Oct. 16]
34. Salerno E P, Bedognetti D, Mauldin I S, et al. Human melanomas and ovarian cancers overexpressing mechanical barrier molecule genes lack immune signatures and have increased patient mortality risk. Oncoimmunology 2016; 5 (12):e1240857. doi: 10.1080/2162402X.2016.1240857 [published Online First: 2017 Jan. 27]
35. Su X, Ma X, Xie X, et al. FN-EDA mediates angiogenesis of hepatic fibrosis via integrin-VEGFR2 in a CD63 synergetic manner. Cell Death Discov 2020; 6 (1): 140. doi: 10.1038/s41420-020-00378-9 [published Online First: 2020 Dec. 10]
36. Xiang L, Xie G, Ou J, et al. The extra domain A of fibronectin increases VEGF-C expression in colorectal carcinoma involving the PI3K/AKT signaling pathway. PLOS One 2012; 7 (4):e35378. doi: 10.1371/journal.pone.0035378 [published Online First: 2012 Apr. 13]
37. Lv W Q, Wang H C, Peng J, et al. Gene editing of the extra domain A positive fibronectin in various tumors, amplified the effects of CRISPR/Cas system on the inhibition of tumor progression. Oncotarget 2017; 8 (62): 105020-36. doi: 10.18632/oncotarget.21136 [published Online First: 2017 Dec. 30]
38. Sengupta S, Nandi S, Hindi E S, et al. Short hairpin RNA-mediated fibronectin knockdown delays tumor growth in a mouse glioma model. Neoplasia 2010; 12 (10): 837-47. doi: 10.1593/neo. 10662 [published Online First: 2010 Oct. 12]
39. Rossnagl S, Altrock E, Sens C, et al. EDA-Fibronectin Originating from Osteoblasts Inhibits the Immune Response against Cancer. PLOS Biol 2016; 14 (9):e1002562. doi: 10.1371/journal.pbio.1002562 [published Online First: 2016 Sep. 23]
40. Liao Y F, Gotwals P J, Koteliansky V E, et al. The EIIIA segment of fibronectin is a ligand for integrins alpha 9beta 1 and alpha 4beta 1 providing a novel mechanism for regulating cell adhesion by alternative splicing. J Biol Chem 2002; 277 (17): 14467-74. doi: 10.1074/jbc.M201100200 [published Online First: 2002 Feb. 13]
41. Sun X, Fa P, Cui Z, et al. The EDA-containing cellular fibronectin induces epithelial-mesenchymal transition in lung cancer cells through integrin alpha9beta1-mediated activation of PI3-K/AKT and Erk1/2. Carcinogenesis 2014; 35 (1): 184-91. doi: 10.1093/carcin/bgt276 [published Online First: 2013 Aug. 10]
42. Manabe R, Ohe N, Maeda T, et al. Modulation of cell-adhesive activity of fibronectin by the alternatively spliced EDA segment. J Cell Biol 1997; 139 (1): 295-307. doi: 10.1083/jcb.139.1.295 [published Online First: 1997 Oct. 6]
43. Okamura Y, Watari M, Jerud E S, et al. The extra domain A of fibronectin activates Toll-like receptor 4. J Biol Chem 2001; 276 (13): 10229-33. doi: 10.1074/jbc.M100099200 [published Online First: 2001 Jan. 21]
44. Nam J M, Onodera Y, Bissell M J, et al. Breast cancer cells in three-dimensional culture display an enhanced radioresponse after coordinate targeting of integrin alpha5beta1 and fibronectin. Cancer Res 2010; 70 (13): 5238-48. doi: 10.1158/0008-5472.CAN-09-2319 [published Online First: 2010 Jun. 3]
45. Liu W T, Jing Y Y, Yu G F, et al. Toll like receptor 4 facilitates invasion and migration as a cancer stem cell marker in hepatocellular carcinoma. Cancer Lett 2015; 358 (2): 136-43. doi: 10.1016/j.canlet.2014.12.019 [published Online First: 2014 Dec. 17]
46. Fu X, Rivera A, Tao L, et al. Genetically modified T cells targeting neovasculature efficiently destroy tumor blood vessels, shrink established solid tumors and increase nanoparticle delivery. Int J Cancer 2013; 133 (10): 2483-92. doi: 10.1002/ijc.28269 [published Online First: 2013 May 11]
47. Byrd T T, Fousek K, Pignata A, et al. TEM8/ANTXR1-Specific CAR T Cells as a Targeted Therapy for Triple-Negative Breast Cancer. Cancer Res 2018; 78 (2): 489-500. doi: 10.1158/0008-5472.CAN-16-1911 [published Online First: 2017 Dec. 1]
48. Petrovic K, Robinson J, Whitworth K, et al. TEM8/ANTXR1-specific CAR T cells mediate toxicity in vivo. PLOS One 2019; 14 (10):e0224015. doi: 10.1371/journal.pone.0224015 [published Online First: 2019 Oct. 18]
49. Zhuang X, Maione F, Robinson J, et al. CAR T cells targeting tumor endothelial marker CLEC14A inhibit tumor growth. JCI Insight 2020; 5 (19) doi: 10.1172/jci.insight. 138808 [published Online First: 2020 Oct. 3]
50. Chinnasamy D, Yu Z, Theoret M R, et al. Gene therapy using genetically modified lymphocytes targeting VEGFR-2 inhibits the growth of vascularized syngenic tumors in mice. J Clin Invest 2010; 120 (11): 3953-68. doi: 10.1172/JCI43490 [published Online First: 2010 Oct. 28]
51. Wagner J, Wickman E, Shaw T I, et al. Antitumor Effects of CAR T Cells Redirected to the EDB Splice Variant of Fibronectin. Cancer Immunol Res 2021; 9 (3): 279-90. doi: 10.1158/2326-6066.CIR-20-0280 [published Online First: 2020 Dec. 24]
52. Xie Y J, Dougan M, Jailkhani N, et al. Nanobody-based CAR T cells that target the tumor microenvironment inhibit the growth of solid tumors in immunocompetent mice. Proc Natl Acad Sci USA 2019; 116 (16): 7624-31. doi: 10.1073/pnas. 1817147116 [published Online First: 2019 Apr. 3]
53. Hartmann J, Schussler-Lenz M, Bondanza A, Buchholz C J. Clinical development of CAR Tcells-challenges and opportunities in translating innovative treatment concepts. EMBO Mol Med 2017; 9:1183-1197.
54. Lamers C H, Klaver Y, Gratama J W, Sleijfer S, Debets R. Treatment of metastatic renal cell carcinoma (mRCC) with CAIX CAR-engineered T-cells-a completed study overview. Biochem Soc Trans 2016; 44:951-959.
55. Parkhurst M R, Yang J C, Langan R C, Dudley M E, Nathan D A, Feldman S A, Davis J L, et al. T cells targeting carcinoembryonic antigen can mediate regression of metastatic colorectal cancer but induce severe transient colitis. Mol Ther 2011; 19:620-626.
56. Jin L, Tao H, Karachi A, Long Y, Hou A Y, Na M, Dyson K A, et al. CXCR1- or CXCR2-modified CAR T cells co-opt IL-8 for maximal antitumor efficacy in solid tumors. Nat Commun 2019; 10:4016.
57. Wang W, Ma Y, Li J, Shi H S, Wang L Q, Guo F C, Zhang J, et al. Specificity redirection by CAR with human VEGFR-1 affinity endows T lymphocytes with tumor-killing ability and antiangiogenic potency. Gene Ther 2013; 20:970-978.
58. Patten S G, Adamcic U, Lacombe K, Minhas K, Skowronski K, Coomber B L. VEGFR2 heterogeneity and response to anti-angiogenic low dose metronomic cyclophosphamide treatment. BMC Cancer 2010; 10:683.
59. Villa A, Trachsel E, Kaspar M, Schliemann C, Sommavilla R, Rybak J N, Rosli C, Borsi L, Neri D. A high-affinity human monoclonal antibody specific to the alternatively spliced EDA domain of fibronectin efficiently targets tumor neo-vasculature in vivo. Int J Cancer. 2008 Jun. 1; 122 (11): 2405-13. doi: 10.1002/ijc.23408.
60. Klebanoff C A, Gattinoni L, Restifo N P. Sorting through subsets: which T-cell populations mediate highly effective adoptive immunotherapy?. J Immunother. 2012; 35 (9): 651-660. doi: 10.1097/CJI.0b013e31827806e6
61. Rivière I, Sadelain M. Chimeric Antigen Receptors: A Cell and Gene Therapy Perspective. Mol Ther. 2017 May 3; 25 (5): 1117-1124. doi: 10.1016/j.ymthe.2017.03.034.
62. Frejd F Y, Kim K T. Affibody molecules as engineered protein drugs. Exp Mol Med. 2017 Mar. 24; 49 (3):e306. doi: 10.1038/emm.2017.35.
63. Carnemolla B, Borsi L, Zardi L, Owens R J, Baralle F E. Localization of the cellular-fibronectin-specific epitope recognized by the monoclonal antibody IST-9 using fusion proteins expressed in E. coli. FEBS Lett. 1987 May 11; 215 (2): 269-73. doi: 10.1016/0014-5793 (87) 80160-6.
64. Gattinoni L, Speiser D E, Lichterfeld M, Bonini C. T memory stem cells in health and disease. Nat Med. 2017 Jan. 6; 23 (1): 18-27. doi: 10.1038/nm.4241.
65. Güler-Gane G, Kidd S, Sridharan S, Vaughan T J, Wilkinson T C, Tigue N J. Overcoming the Refractory Expression of Secreted Recombinant Proteins in Mammalian Cells through Modification of the Signal Peptide and Adjacent Amino Acids. PLoS One. 2016 May 19; 11 (5): e0155340. doi: 10.1371/journal.pone.0155340.65.
66. Winn H J. Immune mechanisms in homotransplantation. II. Quantitative assay of the immunologic activity of lymphoid cells stimulated by tumor homografts. J Immunol 1961; 86:228-39. [published Online First: 1961 Feb. 1]
67. van Dijk F, Olinga P, Poelstra K, et al. Targeted Therapies in Liver Fibrosis: Combining the Best Parts of Platelet-Derived Growth Factor BB and Interferon Gamma. Front Med (Lausanne) 2015; 2:72. doi: 10.3389/fmed.2015.00072 [published Online First: 2015 Oct. 27]
68. Coughlin C M, Salhany K E, Gee M S, et al. Tumor cell responses to IFNgamma affect tumorigenicity and response to IL-12 therapy and antiangiogenesis. Immunity 1998; 9 (1): 25-34. doi: 10.1016/s1074-7613 (00) 80585-3 [published Online First: 1998 Aug. 11
69. Langaas V, Shahzidi S, Johnsen J I, et al. Interferon-gamma modulates TRAIL-mediated apoptosis in human colon carcinoma cells. Anticancer Res 2001; 21 (6A): 3733-8. [published Online First: 2002 Mar. 26]
70. Mazzolini G, Narvaiza I, Martinez-Cruz L A, et al. Pancreatic cancer escape variants that evade immunogene therapy through loss of sensitivity to IFNgamma-induced apoptosis. Gene Ther 2003; 10 (13): 1067-78. doi: 10.1038/sj.gt.3301957 [published Online First: 2003 Jun. 17]
71. Dighe A S, Richards E, Old L J, et al. Enhanced in vivo growth and resistance to rejection of tumor cells expressing dominant negative IFN gamma receptors. Immunity 1994; 1 (6): 447-56. doi: 10.1016/1074-7613 (94) 90087-6 [published Online First: 1994 Sep. 1]
72. Brunda M J, Rosenbaum D. Modulation of murine natural killer cell activity in vitro and in vivo by recombinant human interferons. Cancer Res 1984; 44 (2): 597-601. [published Online First: 1984 Feb. 1]
73. Qin Z, Schwartzkopff J, Pradera F, et al. A critical requirement of interferon gamma-mediated angiostasis for tumor rejection by CD8+ T cells. Cancer Res 2003; 63 (14): 4095-100. [published Online First: 2003 Jul. 23]
74. Quinn K M, Kartikasari A E R, Cooke R E, et al. Impact of age-, cancer-, and treatment-driven inflammation on T cell function and immunotherapy. J Leukoc Biol 2020; 108 (3): 953-65. doi: 10.1002/JLB.5MR0520-466R [published Online First: 2020 Jul. 18]
75. Caetano M S, Zhang H, Cumpian A M, et al. IL6 Blockade Reprograms the Lung Tumor Microenvironment to Limit the Development and Progression of K-ras-Mutant Lung Cancer. Cancer Res 2016; 76 (11): 3189-99. doi: 10.1158/0008-5472.CAN-15-2840 [published Online First: 2016 May 20]
76. Naviaux R K, Costanzi E, Haas M, Verma I M. The pCL vector system: rapid production of helper-free, high-titer, recombinant retroviruses. J Virol. 1996 August; 70 (8): 5701-5. doi: 10.1128/JVI.70.8.5701-5705.1996.
77. Dull T, Zufferey R, Kelly M, Mandel R J, Nguyen M, Trono D, Naldini L. A third-generation lentivirus vector with a conditional packaging system. J Virol. 1998 November; 72 (11): 8463-71. doi: 10.1128/JVI.72.11.8463-8471.1998.
78. Watanabe N, Mckenna M K. Generation of CAR T-cells using γ-retroviral vector. Methods Cell Biol. 2022; 167:171-183. doi: 10.1016/bs.mcb.2021.06.014. Epub 2021 Jul. 30.
79. Rosskopf S, Leitner J, Paster W, Morton L T, Hagedoorn R S, Steinberger P, Heemskerk M H M. A Jurkat 76 based triple parameter reporter system to evaluate TCR functions and adoptive T cell strategies. Oncotarget. 2018 Apr. 3; 9 (25): 17608-17619.
80. Ryan M D, King A M, Thomas G P. Cleavage of Foot-and-Mouth Disease Virus Polyprotein Is Mediated by Residues Located within a 19 Amino Acid Sequence. J Gen Virol (1991) 72 (Pt 11): 2727-32.
81. Spada S, Tocci A, Di Modugno F, Nisticò P. Fibronectin as a multiregulatory molecule crucial in tumor matrisome: from structural and functional features to clinical practice in oncology. J Exp Clin Cancer Res. 2021 Mar. 17; 40 (1):102.
82. Madri J A. Extracellular matrix modulation of vascular cell behaviour. Transpl Immunol 1997; 5 (3): 179-83.

1. A chimeric receptor nucleic acid comprising:
a) a polynucleotide coding for a ligand binding domain comprising an extra-domain A of fibronectin (EDA) targeting-moiety, wherein the EDA targeting moiety is an antibody fragment comprising:
i. a VH domain comprising a HCDR1 consisting of SEQ ID NO: 1, a HCDR2 consisting of SEQ ID NO: 2 and a HCDR3 consisting of SEQ ID NO: 3;
ii. a VL domain comprising a LCDR1 consisting of SEQ ID NO: 4, a LCDR2 consisting of SEQ ID NO: 5 and a LCDR3 consisting of SEQ ID NO: 6;
b) a polynucleotide coding for a transmembrane domain; and
c) a polynucleotide coding for an intracellular signaling domain.

2. The chimeric receptor nucleic acid according to claim 1, wherein the EDA targeting moiety is a scFv comprising a VH domain consisting of SEQ ID NO: 7 and a VL domain consisting of SEQ ID NO: 8.

3. The chimeric receptor nucleic acid according to claim 1, wherein the transmembrane domain comprises the transmembrane domain of CD28, CD3, CD45, CD4, CD8, CD9, CD16, CD22, CD33, CD37, CD64, CD80, CD86, CD134, CD137, or CD154.

4. The chimeric receptor nucleic acid according to claim 1, wherein the intracellular signaling domain comprises the intracellular domain of CD3ζ, FcRγ, CD3γ, CD3δ, CD3ε, CD5, CD22, CD79a, CD79b, or CD66b.

5. The chimeric receptor nucleic acid according to claim 1, wherein the chimeric receptor further comprises a costimulatory signaling domain.

6. The chimeric receptor nucleic acid according to claim 1, wherein, the chimeric receptor comprises:
(i) a scFv comprising a VH domain and VL domain, wherein the VH domain comprises a HCDR1 consisting of SEQ ID NO: 1, a HCDR2 consisting of SEQ ID NO: 2 and a HCDR3 consisting of SEQ ID NO: 3; and the VL domain comprises a LCDR1 consisting of SEQ ID NO: 4, a LCDR2 consisting of SEQ ID NO: 5 and a LCDR3 consisting of SEQ ID NO: 6;
(ii) a hinge or spacer region comprising or consisting of SEQ ID NO: 10;
(iii) a transmembrane domain comprising or consisting of SEQ ID NO: 11;
(iv) an intracellular signaling domain comprising or consisting of SEQ ID NO: 12; and
(v) a costimulatory signaling domain comprising or consisting of SEQ ID NO: 13.

7. A chimeric receptor polypeptide coded for by a chimeric receptor nucleic acid according to claim 1.

8. An expression vector comprising a chimeric receptor nucleic acid according to claim 1.

9. A host cell comprising a nucleic acid according to claim 1 or an expression vector of claim 8.

10. The host cell according to claim 9, wherein the host cell is
a) a CD8+ T lymphocyte cell selected from the group consisting of naïve CD8+ T cells, stem cell memory CD8+ T cells, central memory CD8+ T cells, effector memory CD8+ T cells and bulk CD8+ T cells; and/or
b) a CD4+T lymphocyte cell selected from the group consisting of naïve CD4+ T cells, stem cell memory CD4+ T cells, central memory CD4+ T cells, effector memory CD4+ T cells and bulk CD4+ T cells.

11. A pharmaceutical composition comprising a host cell claim 9, such as a plurality thereof, and a pharmaceutically acceptable excipient, diluent or carrier.

12. (canceled)

13. A method of treating an EDA-positive cancer in a subject wherein said method comprises administering a therapeutically effective amount of a chimeric receptor nucleic acid comprising:
a) a polynucleotide coding for a ligand binding domain comprising an extra-domain A of fibronectin (EDA) targeting-moiety;
b) a polynucleotide coding for a transmembrane domain; and
c) a polynucleotide coding for an intracellular signaling domain; or
a chimeric receptor polypeptide coded for thereby;
an expression vector comprising the chimeric receptor nucleic acid;
a host cell comprising the chimeric receptor nucleic acid or the expression vector; or
a pharmaceutical composition comprising any thereof and further comprising and a pharmaceutically acceptable excipient, diluent or carrier.

14. The method of treating an EDA-positive cancer in a subject according to claim 13, wherein the chimeric receptor nucleic acid comprises the chimeric receptor nucleic acid of claim 1.

15. The method of treating an EDA-positive cancer in a subject according to claim 13, wherein said EDA-positive cancer is colorectal cancer, liver cancer, pancreatic cancer, breast cancer, ovary cancer, prostate cancer, testis cancer, bladder cancer, glioma, melanoma, lymphoma, head and neck cancer, teratocarcinoma, or cholangiocarcinoma.

16. The chimeric receptor nucleic acid according to claim 1, further comprising a polynucleotide coding for a hinge or spacer region.

17. The chimeric receptor nucleic acid according to claim 1, wherein the antibody fragment is a single chain Fv (scFv).

18. The chimeric receptor nucleic acid according to claim 2, wherein said EDA targeting moiety is a scFv comprising or consisting of SEQ ID NO: 9.

19. The chimeric receptor nucleic acid according to claim 3, wherein the transmembrane domain comprises the transmembrane domain of CD8.

20. The chimeric receptor nucleic acid according to claim 4, wherein the intracellular signaling domain comprises the intracellular domain of CD35.

21. The chimeric receptor nucleic acid according to claim 5, wherein the costimulatory signaling domain comprises the intracellular domain of CD27, CD28, 41BB (CD137), CD134, CD30, CD40, lymphocyte function-associated antigen-1 (LFA-1), CD2, CD7, LIGHT, NKG2C, CD278 or CD276.

22. The chimeric receptor nucleic acid according to claim 5, wherein the costimulatory signaling domain comprises the intracellular domain of 41BB (CD137).

23. The chimeric receptor nucleic acid according to claim 6, wherein in the CAR nucleic acid the coding polynucleotide of the EDA-targeting ScFv is operably linked to the coding polynucleotide of the hinge or spacer region which is operably linked to the transmembrane region coding polynucleotide which is operably linked to the polynucleotide coding for the costimulatory signaling domain which is operably linked to the intracellular signaling domain coding polynucleotide.

24. The expression vector according to claim 8, wherein the vector is a viral vector.

25. The expression vector according to claim 24, wherein the viral vector is a γ-retroviral or lentiviral vector.

26. The host cell according to claim 9, wherein the host cell is a T lymphocyte cell or a natural killer (NK) cell.

27. The method of treating an EDA-positive cancer in a subject according to claim 13, wherein the polynucleotide coding for a ligand binding domain comprising an extra-domain A of fibronectin (EDA) targeting-moiety is a single chain Fv (scFv).

28. The method of treating an EDA-positive cancer in a subject according to claim 13, wherein the chimeric receptor nucleic acid further comprises a polynucleotide coding for a hinge or spacer region.

29. The method of treating an EDA-positive cancer in a subject according to claim 13, wherein the chimeric receptor nucleic acid further comprises a polynucleotide coding for a costimulatory signaling domain.
